SEQUENCE LISTING (24 sequences)

SEQ ID NO: 1 - protein, 326 aa, Platymonas (Tetraselmis) subcordiformis
MGFQLNPEYLNETILLDDCTPIYLNVGPLWEQKVARGTQWFGVILSLAFLIYYIWITYKATCGWEELYVCTIEFCKIVIELYFEFSPPAMIYQTNGEVTPWLRYAEWLLTCPVILIHLSNITGLNDDYSGRTMSLITSDLGGICMAVTSALSKGWLKWLFFVIGCCYGASTFYHAALIYIESYYTMPHGVCKNMVLAMAAVFFTSWFMFPGLFLAGPEGTNALSWAGSTIGHTVADLLSKNAWGMIGHFLRLEIHKHIIIHGDVRRPITVNTLGREVTVSCFVDKEEEDEDERISTKTYANRASFMKMRNDMEQRGIQTRKSLEML

SEQ ID NO: 2 - protein, 1000 aa, Platymonas (Tetraselmis) subcordiformis
ATGGGCTTCCAGCTGAACCCGGAGTACCTGAACGAGACGATCCTTCTGGACGACTGTACCCCCATCTACCTCAACGTTGGCCCACTCTGGGAACAGAAAGTCGCGCGCGGGACGCAGTGGTTCGGCGTCATCCTGTCCCTCGCCTTCCTTATCTATTACATATGGATCACGTACAAGGCCACCTGCGGCTGGGAGGAGCTCTACGTGTGCACAATCGAGTTCTGCAAGATTGTCATCGAGCTGTACTTTGAGTTCTCCCCGCCAGCCATGATTTACCAGACCAACGGGGAAGTCACGCCGTGGTTGCGATACGCGGAGTGGCTGCTAACGTGTCCGGTGATCCTAATTCATTTGTCCAACATCACCGGTTTAAACGACGATTACAGCGGCCGCACGATGAGTTTGATAACCTCAGATTTGGGAGGCATCTGTATGGCTGTAACCTCTGCACTCTCGAAAGGATGGCTCAAGTGGTTGTTTTTTGTAATCGGCTGCTGCTACGGCGCCTCCACTTTCTACCACGCTGCTCTCATCTACATCGAGTCCTACTACACCATGCCACACGGAGTCTGCAAAAATATGGTGCTCGCGATGGCCGCTGTCTTCTTCACCTCCTGGTTCATGTTCCCGGGCCTCTTTCTCGCCGGTCCCGAGGGCACAAACGCTCTGTCATGGGCCGGAAGCACAATTGGTCACACGGTGGCCGATCTGCTATCTAAAAACGCATGGGGGATGATCGGACACTTCCTTCGTCTCGAGATCCACAAACATATTATCATCCACGGAGACGTGCGACGCCCGATCACGGTGAACACCCTCGGGCGTGAGGTGACGGTGTCGTGCTTTGTGGACAAGGAGGAGGAGGATGAGGATGAACGCATCTCGACTAAGACTTACGCCAACCGCGCGAGCTTTATGAAGATGAGGAACGACATGGAGCAGCGCGGCATCCAGACGCGCAAGTCTTTGGAAATGCTAGCGCCGCCACCCGCACTGAACG

SEQ ID NO: 3 - protein, 660 aa, Platymonas (Tetraselmis) subcordiformis
MGFQLNPEYLNETILLDDCTPIYLNVGPLWEQKVARGTQWFGVILSLAFLIYYIWITYKATCGWEELYVCTIEFCKIVIELYFEFSPPAMIYQTNGEVTPWLRYAEWLLTCPVILIHLSNITGLNDDYSGRTMSLITSDLGGICMAVTSALSKGWLKWLFFVIGCCYGASTFYHAALIYIESYYTMPHGVCKNMVLAMAAVFFTSWFMFPGLFLAGPEGTNALSWAGSTIGHTVADLLSKNAWGMIGHFLRLEIHKHIIIHGDVRRPITVNTLGREVTVSCFVDKEEEDEDERISTKTYANRASFMKMRNDMEQRGIQTRKSLEMLAPPPALNDGSIVLAVADPMTLTFFTQQLSQLDATIRATPAMGQGQLEQVLEKGGFDGVLVSPEYIQQVGLVQRLKDKYHMPVYAFGWGKSSPWRSVIEGSGVDGWLEGPYFGSTFDTDALSDAIAEMQRIKTSYSMVVNGVGMNGAGMNGVGMNGMGMDGVGMNGAGMNGVGMNGMGGYGSVGNNMHSMPMMSQQAVMMPQSAPQMMGGMSQTQQPAMMGGMQGASPHYSVGNLQNMEGQQAVGSPQVLASSWQQSALHGGMGQQQQYGQVQQMPMMVGMQTPASPGGVQTPPHTMAGQPQMSPQQLQQQLYFMQQLQQRQLQQQQYQGGTGQR

SEQ ID NO: 4 - protein, 365 aa, Dunaliella salina
MRRRESQLAYLCLFVLIAGWAPRLTESAPDLAERRPPSERNTPYANIKKVPNITEPNANVQLDGWALYQDFYYLAGSDKEWVVGPSDQCYCRAWSKSHGTDREGEAAVVWAYIVFAICIVQLVYFMFAAWKATVGWEEVYVNIIELVHIALVIWVEFDKPAMLYLNDGQMVPWLRYSAWLLSCPVILIHLSNLTGLKGDYSKRTMGLLVSDIGTIVFGTSAALAPPNHVKVILFTIGLLYGLFTFFTAAKVYIEAYHTVPKGQCRNLVRAMAWTYFVSWAMFPILFILGREGFGHITYFGSSIGHFILEIFSKNLWSLLGHGLRYRIRQHIIIHGNLTKKNKINIAGDNVEVEEYVDSNDKDSDV

SEQ ID NO: 5 - protein, 352 aa, Chlamydomonas (Chloromonas) augustae
MDTLAWVARELLSTAHDATPATATPSTDHSTPSTDHGSGETFNVTITIGGGHHGGHAGPVDNSIVIGGIDGWIAIPAGDCYCAGWYVSHGSSFEATFAHVCQWSIFAVCILSLLWYAWQYWKATCGWEEVYVCCIELVFICFELYHEFDSPCSLYLSTANIVNWLRYSEWLLCCPVILIHLSNVTGLSDDYGRRTMGLLVSDIATIVFGITAAMLVSWPKIIFYLLGFTMCCYTFYLAAKVLIESFHQVPKGICRHLVKAMAITYYVGWSFFPLIFLFGQSGFKKISPYADVIASSFGDLISKNMFGLLGHFLRVKIHEHILKHGDIRKTTHLRIAGEEKEVETFVEEEDED

SEQ ID NO: 6 - protein, 331 aa, Mesostigma viride
MSPPTSPTPDTGHDTPDTGHDTGGHGAVEICFAPCEEDCVTIRYFVENDFEGCIPGHFDQYSSHGSLHDIVKAALYICMVISILQILFYGFQWWRKTCGWEVWFVACIETSIYIIAITSEADSPFTLYLTNGQISPQLRYMEWLMTCPVILIALSNITGMAEEYNKRTMTLLTSDVCCIVLGMMSAASKPRLKGILYAVGWAFGAWTYWTALQVYRDAHKAVPKPLAWYVRAMGYVFFTSWLTFPGWFLLGPEGLEVVTGTVSTLMHACSDLISKNLWGFMDWHLRVLVARHHRKLFKAEEEHALKKGQTLEPGMPRSTSFVRGLGDDVEI

SEQ ID NO: 7 - protein, 309 aa, Chlamydomonas reinhardtii
MDYGGALSAVGRELLFVTNPVVVNGSVLVPEDQCYCAGWIESRGTNGAQTASNVLQWLAAGFSILLLMFYAYQTWKSTCGWEEIYVCAIEMVKVILEFFFEFKNPSMLYLATGHRVQWLRYAEWLLTCPVILIHLSNLTGLSNDYSRRTMGLLVSDIGTIVWGATSAMATGYVKVIFFCLGLCYGANTFFHAAKAYIEGYHTVPKGRCRQVVTGMAWLFFVSWGMFPILFILGPEGFGVLSVYGSTVGHTIIDLMSKNCWGLLGHYLRVLIHEHILIHGDIRKTTKLNIGGTEIEVETLVEDEAEAGAV

SEQ ID NO: 8 - protein, 355 aa, Chlamydomonas yellowstonensis
MDTLAWVARELLSSGHGTDTATDSGHGTDTSGGHDSSHDAVAHNVTLLIAPPHAGGHAGPTDTSQQITGIDGWIAIPAGDCYCAGWYVSHGSSFEATFAHVCQWSIFAVCVLSLLWYAYQYWKATCGWEEVYVCCIELVFICFELYHEFDSPCSLYLSTSNVVNWLRYSEWLLCCPVILIHLSNVTGLSDDYGRRTMGLLVSDIATIVFGVTAAMLVNWPKIIFYLIGFTMCCYTFFLAAKVLIESFHQVPKGICRHLVKAMAITYFVGWSFFPLIFLFGQSGFKKISPYADVIASSFGDLISKNAFGMLGHFLRVKIHEHILKHGDIRKTTHLRIAGEEKEVETFVEEEDEDTA

SEQ ID NO: 9 - protein, 635 aa, Chlamydomonas raudensis
MASMAFSAISLASSAMRSLQASGGNPFEHDAPPDNSCELTPYGCLNDFYCNPAYGLADAGYNYCYVQSAYGKLAIVQTDQLSWLYSHGSSGAKAASIAFQWLAFATAVIGLMFYAWDTWRATTGWEEVYVCTIELIKVLIEIFKEFEFPCSLYLPTGNWVLWLRYAEWLLTCPVILIHLSNITGLKDDYNKRTMRLLVSDIGCIVWGVTSAMTAGYLKWIFFAIGLLYGSNTYFHSAKVYIEAYHTVPKGRRRVIVRLMAYCFYLAWTMFPILFALGPEGMGQMSAYMSTILTTIADVLSKQIWGLLGHHLRVKIYQHILIHGDIRKKTTMQVGGEDVEVEEFVDEDDEEGVRQANTQLANRESFVHMAEQMKKNGIEVRATYDTGVDKEMGHHHVEAGRIILAVPDMSMVDFFRQQLSQMPAPIELVPALGIDNTVQLVQQAAALGGCDFVLVHPEFLKDASSSGLVTKLRMMGQRVCAFGWSPMGPQRELIESRGLDGWLEGPSFGSGIDRHQLTALVSRMQMMRKATMGSGMANPMAQQQQSFMMHQQNSAHNSFMIQPQTQPQANPLYGAQMGSQMGATTGSALFHPAAPGNATPPSPSGAANVNEAEMLQQLMGEITRLKSELGGSGTPR

SEQ ID NO: 10 - protein, 712 aa, Chlamydomonas reinhardtii
MSRRPWLLALALAVALAAGSAGASTGSDATVPVATQDGPDYVFHRAHERMLFQTSYTLENNGSVICIPNNGQCFCLAWLKSNGTNAEKLAANILQWITFALSALCLMFYGYQTWKSTCGWEEIYVATIEMIKFIIEYFHEFDEPAVIYSSNGNKTVWLRYAEWLLTCPVILIHLSNLTGLANDYNKRTMGLLVSDIGTIVWGTTAALSKGYVRVIFFLMGLCYGIYTFFNAAKVYIEAYHTVPKGICRDLVRYLAWLYFCSWAMFPVLFLLGPEGFGHINQFNSAIAHAILDLASKNAWSMMGHFLRVKIHEHILLYGDIRKKQKVNVAGQEMEVETMVHEEDDETQKVPTAKYANRDSFIIMRDRLKEKGFETRASLDGDPNGDAEANAAAGGKPGMEMGKMTGMGMSMGAGMGMANIDSGRVILAVPDISMVDFFREQFARLPVPYELVPALGAENTLQLVQQAQSLGGCDFVLMHPEFLRDRSPTGLLPRLKMGGQRAAAFGWAAIGPMRDLIEGSGVDGWLEGPSFGAGINQQALVALINRMQQAKKMGMMGGMGMGMGGGMGMGMGMGMGMAPSMNAGMTGGMGGASMGGAVMGMGMGMQPMQQAMPAMSPMMTQQPSMMSQPSAMSAGGAMQAMGGVMPSPAPGGRVGTNPLFGSAPSPLSSQPGISPGMATPPAATAAPAAGGSEAEMLQQLMSEINRLKNELGE

SEQ ID NO: 11 - protein, 737 aa, Chlamydomonas reinhardtii
MDYGGALSAVGRELLFVTNPVVVNGSVLVPEDQCYCAGWIESRGTNGAQTASNVLQWLAAGFSILLLMFYAYQTWKSTCGWEEIYVCAIEMVKVILEFFFEFKNPSMLYLATGHRVQWLRYAEWLLTCPVILIHLSNLTGLSNDYSRRTMGLLVSDIGTIVWGATSAMATGYVKVIFFCLGLCYGANTFFHAAKAYIEGYHTVPKGRCRQVVTGMAWLFFVSWGMFPILFILGPEGFGVLSVYGSTVGHTIIDLMSKNCWGLLGHYLRVLIHEHILIHGDIRKTTKLNIGGTEIEVETLVEDEAEAGAVNKGTGKYASRESFLVMRDKMKEKGIDVRASLDNSKEVEQEQAARAAMMMMNGNGMGMGMGMNGMNGMGGMNGMAGGAKPGLELTPQLQPGRVILAVPDISMVDFFREQFAQLSVTYELVPALGADNTLALVTQAQNLGGVDFVLIHPEFLRDRSSTSILSRLRGAGQRVAAFGWAQLGPMRDLIESANLDGWLEGPSFGQGILPAHIVALVAKMQQMRKMQQMQQIGMMTGGMNGMGGGMGGGMNGMGGGNGMNNMGNGMGGGMGNGMGGNGMNGMGGGNGMNNMGGNGMAGNGMGGGMGGNGMGGSMNGMSSGVVANVTPSAAGGMGGMMNGGMAAPQSPGMNGGRLGTNPLFNAAPSPLSSQLGAEAGMGSMGGMGGMSGMGGMGGMGGMGGAGAATTQAAGGNAEAEMLQNLMNEINRLKRELGE

SEQ ID NO: 12 - protein, 717 aa, Chlamydomonas yellowstonensis
MDTLAWVARELLSSGHGTDTATDSGHGTDTSGGHDSSHDAVAHNVTLLIAPPHAGGHAGPTDTSQQITGIDGWIAIPAGDCYCAGWYVSHGSSFEATFAHVCQWSIFAVCVLSLLWYAYQYWKATCGWEEVYVCCIELVFICFELYHEFDSPCSLYLSTSNVVNWLRYSEWLLCCPVILIHLSNVTGLSDDYGRRTMGLLVSDIATIVFGVTAAMLVNWPKIIFYLIGFTMCCYTFFLAAKVLIESFHQVPKGICRHLVKAMAITYFVGWSFFPLIFLFGQSGFKKISPYADVIASSFGDLISKNAFGMLGHFLRVKIHEHILKHGDIRKTTHLRIAGEEKEVETFVEEEDEDTAKHSTKELANRGSFIVMRDKMKEQGIDVRASLDMDEDEEARTGKGKGAGATSLVPGRVILAVPDISMVDFFHDHFAHLGASIELVPALGVENTLLLVQQAMQLGGLDFVLVHPEFLRDRSQNGLVSRLKMTGHGVCAFGWVPSGPMREIIESAGVDGWLDGPSFGTGIDQEQLIELIGYMQAKRKFGMRFGGGGASKAGYSSDGGFGGKGMLEMQPSMSQGSGVPLLQQNNSMMRAPPSPMGNMANNGMMNPMMSMNNPMMGGGAVMMTSMGSMQQAANPLYGAPPSPLSSQPGAGMYGAPAQPQMGSQGSMHGSMYGGSQQQHQQPQQAAAAPAAADGGSEAEMLKQLMSEINRLKAELGES

SEQ ID NO: 13 - protein, 715 aa, Chlamydomonas (Chloromonas) augustae
MDTLAWVARELLSTAHDATPATATPSTDHSTPSTDHGSGETFNVTITIGGGHHGGHAGPVDNSIVIGGIDGWIAIPAGDCYCAGWYVSHGSSFEATFAHVCQWSIFAVCILSLLWYAWQYWKATCGWEEVYVCCIELVFICFELYHEFDSPCSLYLSTANIVNWLRYSEWLLCCPVILIHLSNVTGLSDDYGRRTMGLLVSDIATIVFGITAAMLVSWPKIIFYLLGFTMCCYTFYLAAKVLIESFHQVPKGICRHLVKAMAITYYVGWSFFPLIFLFGQSGFKKISPYADVIASSFGDLISKNMFGLLGHFLRVKIHEHILKHGDIRKTTHLRIAGEEKEVETFVEEEDEDTVKHSTKELANRGSFIVMRGNMKAQGIDVRASLDMEEDEEGGMGKGKGKGAGAASLMPGRVILAVPDISMVDFFHDHFAHLGASIELVPALGVENTLLLVQQAMQLGGLDFVLVHPEFLRDRSQNGLVSRLKMTGHGVCAFGWVPSGPMREIIESAGVDGWLDGPSFGTGIDQEQLIELIGYMQAKRKFSNRFGGGGGGGKPGFASGGGFGGKSGLELAPSMSQGSGVPLLQQQNSMMRAPPSPMPNNGMMNPMMNPMMGAGGGNIMMTNMGGMNQAANPLYGAPPSPLSSQPGAGMYGAPQQPQMGSQGSMYNSGSQLQHQQSMQQQQQAAPAPAADGGSEAEMLKQLMSEINRLKAELGES

SEQ ID NO: 14 - protein, 593 aa, Mesostigma viride
MSPPTSPTPDTGHDTPDTGHDTGGHGAVEICFAPCEEDCVTIRYFVENDFEGCIPGHFDQYSSHGSLHDIVKAALYICMVISILQILFYGFQWWRKTCGWEVWFVACIETSIYIIAITSEADSPFTLYLTNGQISPQLRYMEWLMTCPVILIALSNITGMAEEYNKRTMTLLTSDVCCIVLGMMSAASKPRLKGILYAVGWAFGAWTYWTALQVYRDAHKAVPKPLAWYVRAMGYVFFTSWLTFPGWFLLGPEGLEVVTGTVSTLMHACSDLISKNLWGFMDWHLRVLVARHHRKLFKAEEEHALKKGQTLEPGMPRSTSFVRGLGDDVEIDPSYELYRLKRQNHPEYFLSPAQTPRRGPSFDKRTSFEMDGGKNGMLQMMPVTGMGMGMGMGMGGGKTVLFLDYTGGGYVSFFEQQLSNMGVNVTKCWSDDDMYNTAGVANVKQLFHFAMIPNNALGGQMVMDLRGTGLLVVAYGPEPPMPGMGQDEFVPLQMPGVPYDESILHNLVMRHAITQGLGMNGMQGNMGQQQQMMGMQGNMNGMQGNMNGMQGNMNGMQGNMSGMQGNMNGMQGNSGMNQGWNNQGFTNTGAFGY

SEQ ID NO: 15 - protein, 677 aa, Haematococcus pluvialis
MRICLGLLAVALATLTLPCPPVNASLSKFKLFFGNSPGLVPNTTVAYGEELYRGFHQLEANGDWVVGPRDSCYCEKWAKSHGTKDEKLGAIVAMFIVFGSCVGALIFYGVAAWRTTCGWEEVYVCVIELAHVLIAIFHEIESPSTLYLSTGNQVLWLRYAEWLLSCPVILIHLSNLTGMKDDYSKRTMGLLISDIGTIVFGTTAAMSPPNYLKVIFWFCGLSYGVTTFYLAAKVYIEAYHTVPKGICRKIVRFMAWDYFGSWCMFPILFVLGPEGFGHISAYGSVIAHQVLDITSKNIWSMAGHFLRVKIHEHIIVHGNITKKTKITLAGEPVEVEEYIDSNEVDPAEMDQVQDKGTQALAHRQSFLVMRNRMQQKGVEVRASLDNSDRDQTENGMGGGAVTMGQWGMGNKAVGDPGAMGLGMVTKAVEEPAEKMGPLEPGRVILCMPDVALVDFWRLQFSSLPAPFEVYPAVGPDMTLQLVQQSLQLGGPSFCDFVLVAPDFLQNPSPSGLVGRLKMMGMRVAAYGWQPAGPMRQLVETSNVDGFLEGPTAPHGVNRSQFSALVFRMQQLKKMASMAALGAPPSMLPPTTSQHSTAVLASSVPNPLQSDSIPAAFPGQGLGTPTTANPLFSVMNSSPPSPLSSRPTVPAPMGAEGEAALMQQLMAEINQLRAELQQ

SEQ ID NO: 16 - protein, 300 aa, Volvox carteri
MDYPVARSLIVRYPTDLGNGTVCMPRGQCYCEGWLRSRGTSIEKTIAITLQWVVFALSVACLGWYAYQAWRATCGWEEVYVALIEMMKSIIEAFHEFDSPATLWLSSGNGVVWMRYGEWLLTCPVLLIHLSNLTGLKDDYSKRTMGLLVSDVGCIVWGATSAMCTGWTKILFFLISLSYGMYTYFHAAKVYIEAFHTVPKGICRELVRVMAWTFFVAWGMFPVLFLLGTEGFGHISPYGSAIGHSILDLIAKNMWGVLGNYLRVKIHEHILLYGDIRKKQKITIAGQEMEVETLVAEEED

SEQ ID NO: 17 - protein, 747 aa, Volvox carteri
MDHPVARSLIGSSYTNLNNGSIVIPSDACFCMKWLKSKGSPVALKMANALQWAAFALSVIILIYYAYATWRTTCGWEEVYVCCVELTKVVIEFFHEFDEPGMLYLANGNRVLWLRYGEWLLTCPVILIHLSNLTGLKDDYNKRTMRLLVSDVGTIVWGATAAMSTGYIKVIFFLLGCMYGANTFFHAAKVYIESYHTVPKGLCRQLVRAMAWLFFVSWGMFPVLFLLGPEGFGHLSVYGSTIGHTIIDLLSKNCWGLLGHFLRLKIHEHILLYGDIRKVQKIRVAGEELEVETLMTEEAPDTVKKSTAQYANRESFLTMRDKLKEKGFEVRASLDNSGIDAVINHNNNYNNALANAAAAVGKPGMELSKLDHVAANAAGMGGIADHVATTSGAISPGRVILAVPDISMVDYFREQFAQLPVQYEVVPALGADNAVQLVVQAAGLGGCDFVLLHPEFLRDKSSTSLPARLRSIGQRVAAFGWSPVGPVRDLIESAGLDGWLEGPSFGLGISLPNLASLVLRMQHARKMAAMLGGMGGMLGSNLMSGSGGVGLMGAGSPGGGGGAMGVGMTGMGMVGTNAMGRGAVGNSVANASMGGGSAGMGMGMMGMVGAGVGGQQQMGANGMGPTSFQLGSNPLYNTAPSPLSSQPGGDASAAAAAAAAAAATGAASNSMNAMQAGGSVRNSGILAGGLGSMMGPPGAPAAPTAAATAAPAVTMGAPGGGGAAASEAEMLQQLMAEINRLKSELGE

SEQ ID NO: 18 - protein, 217 aa, Pleodorina starrii
GTNAEKLAANILQWGALALCVMFSGYQTWKSTCGWEEIYVAKIEMIKFIIEYFHEFDEPAVIYSFNGNKTVWLRYAEWLLTCVILIHLSNLTGLANDYNNRTMGLLVSDVGTSVWGTTAALSKGYSPCHFLPDLAYGIYTFFNAAKVYIEAYHTVPKGICRDLVRYLAWLYFCSWASFPVLFLLGPEGFGHINQFNSAIAHAILDLASKNAWSVIGH

SEQ ID NO: 19 - protein, 227 aa, Pleodorina starrii
GTNGAQTASNVLQWQLAAGFSILLLMFYAYQTWKSTCGWEEIYVCAIEMVKVILEFFFEFKNPSMLYLATGHRVQWLRYAEWLLTCPVILIHLSNLTGLSNDDSSRTMGLLACSIGTIVWGATSAMASGYVKVIFFCLGVYCANTFYRAQAYIKGYHTVPKGRCRQVVTGMAWLFFVSWGMFPILFILGPEGFGVLSVYGSTVGHTIIDPMSKNRCGLPGHYPRVLV

SEQ ID NO: 20 - protein, 276 aa, Pyramimonas gelidicola
MKPEETDEWIAYSAMWAALVFSLLSGVFYAREYHMKRCGWEVMYVATLETLNYVFLIGWGDLAPFHWKLDTTCHGEDDHSGCWDDPLRDRKINEVPVARYMEWLITCPVLLIALSNLTGLKDDYNWRTMKLITADQGTILFGATGALATGTVKPVFFCVGLMYGGVTYFTALEVYVESYANVPPNAKKTVSIMAYMFFLSWMCFPLFWLLGQEGFGHVNQHVSTTLHAIADLFSKNLWGLYSWYLRLQVREYHRKKWHEEQERLNQGEKEFEIPKE

SEQ ID NO: 21 - DNA, 20 bp, ARTIFICIAL SEQUENCE
  SYNTHETIC
tgcggntgggaggagrtnta

SEQ ID NO: 22 - DNA, 22 bp, ARTIFICIAL SEQUENCE
  SYNTHETIC
kccctcrgkbcccarbaggaas

SEQ ID NO: 23 - DNA, 26 bp, ARTIFICIAL SEQUENCE
  SYNTHETIC
tacgsngantggytnctnacntgccc

SEQ ID NO: 24 - DNA, 25 bp, ARTIFICIAL SEQUENCE
  SYNTHETIC
gagggacgcgcgtgtttgagtgccg

(19) United States
(12) Patent Application Publication
(10) Pub. No.: US 2017/0073380 A1
(43) Pub. Date: Mar. 16, 2017

(54) HIGH-EFFICIENCY, SODIUM-SPECIFIC, BLUE-SHIFTED CHANNELRHODOPSINS

(71) Applicant: BOARD OF REGENTS OF THE UNIVERSITY OF TEXAS SYSTEM, Austin, TX (US)

(72) Inventors: John Lee SPUDICH, Houston, TX (US); Elena G. GOVORUNOVA, Houston, TX (US); Oleg A. SINESHCHEKOV, Houston, TX (US)

(21) Appl. No.: 15/365,189

(22) Filed: Nov. 30, 2016

Related U.S. Application Data
(63) Continuation of application No. 14/625,307, filed on Feb. 18, 2015, now abandoned.
(60) Provisional application No. 61/941,204, filed on Feb. 18, 2014.

(51) Int. Cl. C07K 14/405

(52) U.S. Cl. CPC C07K 14/405; A61K 48/00

(57) ABSTRACT

Methods and compositions used to identify and characterize a new channelrhodopsin derived from algae which is highly efficient, sodium specific and blue-shifted. The rhodopsin domain of this channelrhodopsin can be cloned and expressed in mammalian systems and thus used in optogenetic applications and as therapeutic agents.

### CROSS-REFERENCE TO RELATED APPLICATIONS

This application claims priority to, and is a continuation application of U.S. application Ser. No. 14/625,307, filed Feb. 18, 2015, which is a non-provisional application that claims priority to U.S. Provisional Application Ser. No. 61/941,204, filed Feb. 18, 2014, which is herein incorporated in its entirety by reference.

### STATEMENT REGARDING FEDERALLY SPONSORED RESEARCH OR DEVELOPMENT

This invention was made with U.S. Government support under Grant Nos. RC1AG035779, R37GM027750 and R21MH098288 awarded by the National Institutes of Health. The government has certain rights in the invention.

### TECHNICAL FIELD

This disclosure generally relates to methods and compositions that utilize channelrhodopsins derived from algae, and more particularly to such channelrhodopsins having improved characteristics, such as blue-shifted, for optogenetic applications or for use as therapeutic agents.

### BACKGROUND

Optogenetics (Deisseroth. Nat Methods 8 (1): 26-9, 2011), refers to using new high-speed optical methods for probing and controlling genetically targeted neurons within intact neural circuits. Optogenetics involves the introduction of light-activated channels and enzymes that allow manipulation of neural activity with millisecond precision while maintaining cell-type resolution through the use of specific targeting mechanisms. Because the brain is a high-speed system, millisecond-scale temporal precision is central to the concept of optogenetics, which allows probing the causal role of specific action potential patterns in defined cells.

Light control of motility behavior (phototaxis and photophobic responses) in green flagellate algae is mediated by sensory rhodopsins homologous to phototaxis receptors and light-driven ion transporters in prokaryotic organisms. In the phototaxis process, excitation of the algal sensory rhodopsins leads to generation of transmembrane photoreceptor currents. When expressed in animal cells, the algal phototaxis receptors function as light-gated cation channels, which has earned them the name “channelrhodopsins”. Channelrhodopsins have become useful molecular tools for light control of cellular activity.

Originally, the source of these light-activated channels and enzymes were several microbial opsins, including, Channelrhodopsin-2 (ChR2) a single-component light-activated cation channel from algae, which allowed millisecond-scale temporal control in mammals, required only one gene to be expressed in order to work, and responded to visible-spectrum light with a chromophore (retinal) that was already present and supplied to ChR2 by the mammalian brain tissue. The experimental utility of ChR2 was quickly proven in a variety of animal models ranging from behaving mammals to classical model organisms such as flies, worms, and zebrafish, and hundreds of groups have employed ChR2 and related microbial proteins to study neural circuits.

Described herein is the isolation and characterization of a novel high-efficiency, sodium-specific, blue-shifted channelrhodopsin and its recombinant expression in mammalian cells which demonstrates particular advantages and utility for use in, among other things, optogenetics.

### SUMMARY

The presently disclosed methods and compositions are based, in part, on the discovery and identification of a novel channelrhodopsin, which is blue-shifted, derived from algae that when cloned and expressed by mammalian cells were active for light-activation of neuron firing. The use of these channelrhodopsins would improve optogenetic techniques and applications and they can be used to aid in diagnosis, prevention, and/or treatment of neuronal or neurologic disorders, such as but not limited to Parkinson's disease, as well as for ocular disorders. Also described are methods and compositions of red-shifting the absorbance maxima of channelrhodopsins.

In some embodiments herein is disclosed a recombinant nucleic acid operatively linked to a heterologous promoter sequence, said recombinant nucleic acid comprising: a sequence that encodes a peptide with at least 70% homology to an amino acid sequence selected from SEQ ID NO: 1 or SEQ ID NO: 3; or a sequence that encodes a peptide comprising 225 contiguous amino acids selected from SEQ ID NO: 1 or SEQ ID NO: 3; or a sequence that hybridizes to the nucleotide sequence of SEQ ID NO:2, or the complement thereof. In another embodiment the recombinant nucleic acid comprises an expression vector. In another embodiment of the the recombinant nucleic acid the sequence that hybridised to the nucleotide sequence of SEQ ID NO:2, or the complement thereof, further comprises hybridization to filter-bound DNA in 0.5 M NaHPO4, 7% sodium dodecyl sulfate, 1 mM EDTA at 65° C., and washing in 0.2×SSC/0.1% SDS at 42° C. In a further embodiment a recombinant host cell comprising a recombinant nucleic acid operatively linked to a heterologous promoter sequence, said recombinant nucleic acid comprising: a sequence that encodes a peptide with at least 70% homology to an amino acid sequence selected from SEQ ID NO: 1 or SEQ ID NO: 3; or a sequence that encodes a peptide comprising 225 contiguous amino acids selected from SEQ ID NO: 1 or SEQ ID NO: 3; or a sequence that hybridizes to the nucleotide sequence of SEQ ID NO:2, or the complement thereof is disclosed.

In some embodiments the host cell is an: isolated human cell; a non-human mammalian cell; a bacterial cell; a yeast cell; an insect cell; or a plant cell. In some embodiments a method of restoring photosensitivity to a retina of a subject suffering from vision loss or blindness is disclosed, said method comprising: delivering to the retina of said subject an expression vector comprising a polynucleotide that encodes an amino acid sequence selected from the group consisting of SEQ ID NO: 1 or SEQ ID NO: 3 which encodes a rhodopsin domain of a channelrhodopsin expressible in a retinal neuron; and expressing said vector in said retinal neuron, wherein the expressed rhodopsin renders said retinal neuron photosensitive, thereby restoring photosensitivity to said retina or a portion thereof. In another embodiment the method comprises: delivering to the retina of said subject an expression vector that encodes a rhodopsin domain; said vector comprising an open reading frame encoding the rhodopsin domain of a channelrhodopsin selected from SEQ ID NO: 1, SEQ ID NO: 2 or SEQ ID NO: 3, operatively linked to a promoter sequence; and expressing said vector in said retinal neuron, wherein the expressed rhodopsin renders said retinal neuron photosensitive, thereby restoring photosensitivity to said retina or a portion thereof. In a further embodiment the subject is mammalian, and in a still further embodiment the subject is human. In an embodiment of the method of restoring photosensitivity to a retina o a subject suffering from vision loss or blindness is disclosed, said method comprising: delivering to the retina of said subject an expression vector wherein the delivering comprises a pharmaceutically acceptable carrying agent.

In one embodiment, a light gated channel comprising a peptide sequence with at least 95% homology to SEQ ID NO: 1 or SEQ ID NO: 3 is described. In another embodiment of the light gated channel the peptide sequence is that of SEQ ID NO: 1 or SEQ ID NO: 3. In a further embodiment, a vector comprising a polynucleotide sequence that encodes a peptide sequence operatively linked to a heterologous promoter sequence is disclosed; in another embodiment of the vector, the peptide sequence is SEQ ID NO: 1 or SEQ ID NO: 3. In a further embodiment a recombinant host cell is disclosed that comprising the vector that comprises a polynucleotide sequence that encodes a peptide sequence operatively linked to a heterologous promoter sequence, in another embodiment the host cell is an isolated human cell; a non-human mammalian cell; a bacterial cell; a yeast cell; an insect cell; or a plant cell.

In another embodiment, a method of restoring photosensitivity to a retina of a subject suffering from vision loss or blindness is disclosed wherein the method comprises delivering to the retina of the subject the vector, wherein the vector encodes a rhodopsin domain of a channel rhodopsin expressible in a retinal neuron; expressing the vector in the retinal neuron, wherein the expressed rhodopsin renders the retinal neuron photosensitive, and restores photosensitivity to the retina or a portion thereof. In a further embodiment, a method of restoring photosensitivity to a retina of a subject suffering from vision loss or blindness is disclosed, the method comprising: delivering to the retina of the subject a vector comprising the polynucleotide sequence that encodes a peptide sequence of SEQ ID NO: 1 or SEQ ID NO: 3, operatively linked to a heterologous promoter sequence which encodes a rhodopsin domain of a channel rhodopsin expressible in a retinal neuron; and expressing the vector in the retinal neuron, wherein the expressed rhodopsin renders the retinal neuron photosensitive, and restores photosensitivity to the retina or a portion thereof. In some embodiment the subject is mammalian, in other embodiments the subject is human. In some embodiments, delivering comprises a pharmaceutically acceptable carrying agent.

### BRIEF DESCRIPTION OF THE DRAWINGS

FIG. 1: depicts photoelectric signals generated by the wild-type proton pump AR-3 (above zero line) and its D95N mutant (below zero line) in E. coli suspensions (dashed lines) and HEK293 cells (solid lines) in response to a 6-ns laser flash (532 nm). The signals recorded in E. coli suspensions were normalized to those measured in HEK cells. The arrows show a shift of the peak times and a decrease in the relative amplitude of the negative phase caused by a lower time resolution of measurements in HEK cells compared to those in E. coli suspensions.

FIG. 2: (A-C), depicts (A) electrical currents generated by CaChR1 expressed in an HEK293 cell in response to a laser flash at the holding potential applied in 20-mV increments from −60 to 20 mV (bottom to top). (B and C) Decomposition of the net signals recorded at 20 mV (B) and −20 mV (C) in two components. Shown are the recorded traces (black lines), the properly scaled fast positive current, obtained by measuring the signal at 0 mV (red lines), and the channel current, revealed by subtraction of the fast current from the net signal (blue lines). The smooth lines are the results of fitting of each of the two components with two exponentials.

FIG. 3: (depicts voltage dependence of the mean current measured between 50 and 150 ms (open squares) and between 0.75 and 1.55 ms (open circles) after the flash, and of the amplitude of the fast positive current (solid squares), corrected for contribution of channel current. Data are represented as the mean 5 SE of experiments in 6 cells.

FIG. 4: (A-B): depicts (A) spectral dependence of flash-induced absorbance changes in detergent-purified wild-type CaChR1. (B) Absorbance changes monitored at 390 nm in wild-type CaChR1 (black), CaChR1_E169Q (red), and CaChR1_D299N (green) in response to a 6-ns laser flash (532 nm).The traces in the main figure were arbitrarily scaled for better visualization of changes in the rates of the M intermediate accumulation. (Inset) Relative yields of the M formation.

FIG. 5 (A-B): illustrates Electrical currents recorded from wild-type CaChR1 (black lines) and its E169Q (red lines) and D299N (green lines) mutants expressed in HEK293 cells at the reversal potential for channel currents to minimize their contribution to the signal kinetics (A), and at −60 mV holding potential (B).

FIG. 6 (A-C): illustrates (A) Electrical currents generated by VcChR1 expressed in an HEK293 cell in response to a laser flash at the holding potential applied in 20-mV increments from −60 to 40 mV. (B and C) Electrical currents recorded from wild-type VcChR1 (black lines) and its E118Q (red lines) and D248N (green lines) mutants expressed in HEK293 cells at the reversal potential for channel currents to minimize their contribution to the signal kinetics (B), and at −60 mV holding potential (C).

FIG. 7 (A-C): illustrates (A) Electrical currents generated by DsChR1 expressed in a HEK293 cell in response to a laser flash at the holding potentials indicated in the figure legend. (B and C) Electrical currents recorded from wild-type DsChR1 (red lines) and its A178E (black lines) and A178E_E309Q (green lines) mutants expressed in HEK293 cells at the reversal potential for channel currents to minimize their contribution to the signal kinetics (B), and at −60 mV holding potential (C).

FIG. 8 (A-B): illustrates electrical currents recorded from CrChR2, MvChR1, and PsChR in HEK293 cells in response to a laser flash (solid lines). For comparison, traces from CaChR1 are shown (dashed line). Traces were recorded at the reversal potential for channel currents to minimize their contribution to the signal kinetics (A), and at −60 mV holding potential (B).

FIG. 9 (A-B) illustrates the effect of neutral residue substitution of the Asp85 (A) and Asp212 (B) homologs on peak proton transfer currents (hatched bars) and the ratio between peak channel currents and peak proton transfer currents (black bars) in CaChR1, VcChR1, and DsChR1. The channel currents were measured at −60 mV and the proton transfer currents at the Vᵣ for channel currents. The data are mean values (n ¼ 3-8 cells) measured upon neutralization of the corresponding carboxylate residues normalized to those in the wild-type for CaChR1 and VcChR1, and in the A178E mutant for DsChR1.

FIG. 10: illustrates the relationship between the amplitudes of fast and channel currents generated by wild-type channelrhodopsins. The channel currents were measured at −60 mV and the fast currents at the Vᵣ for channel currents. The data are mean values (n ¼ 3-8 cells).

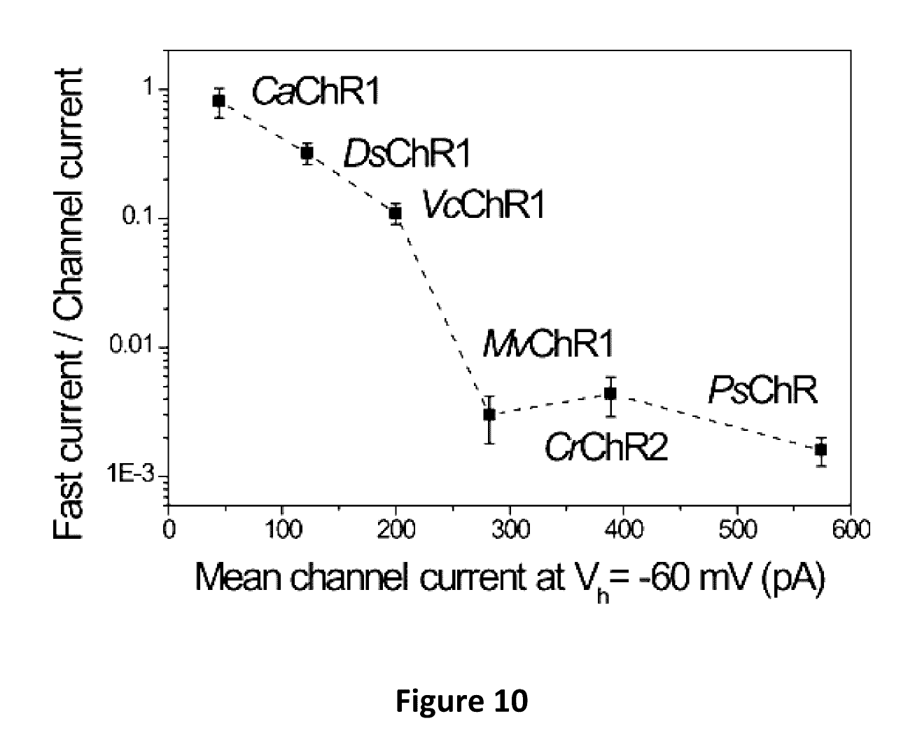

FIG. 11 (A-B): illustrates currents generated by CaChR1 in HEK293 cells in response to laser flashes of (A) different intensity (black line, 6 mW and red line, 0.08 mW), and (B) different time interval between successive flashes (black line, 30 s and red line, 2 s). Signals were normalized at the peak value of the channel current.

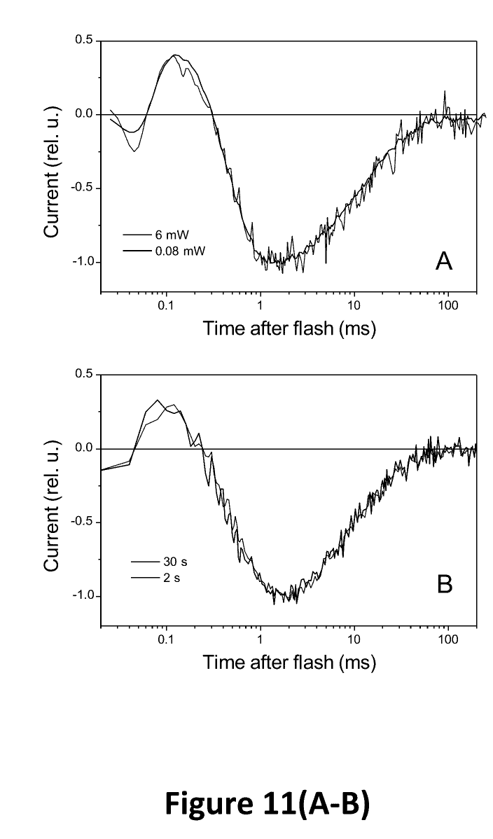

FIG. 12: illustrates a differential signal calculated by subtraction of traces recorded at −60 and 20 mV upon laser flash excitation of CaChR1 expressed in HEK293 cells.

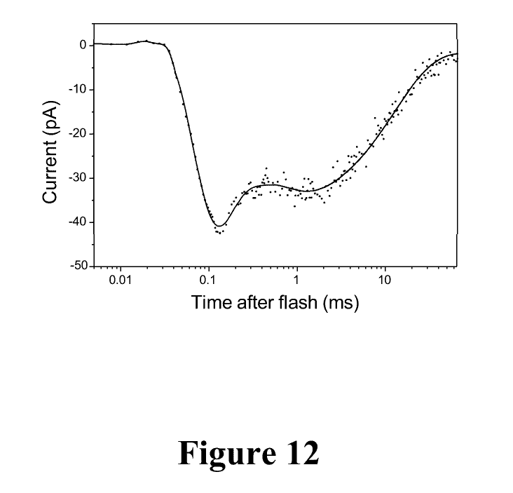

FIG. 13: illustrates absorbance changes at 510 nm of CaChR1 in response to a laser flash (532 nm) in intact Pichia membranes (black lines) and in detergent (red lines). The maximal absolute absorbance change in membranes was ˜1 mOD. Absorbance changes in purified pigment were arbitrarily scaled for better visualization of the difference in the rates of photocycles. dependence of the rate of the fast decay component on the external pH of MChR1 from M. viride and VChR1 from V. carteri (filled squares and open circles, respectively) expressed in HEK293 cells. Excitation: 530 nm, 2 s. Data are the mean values±SEM of 6 to 10 cells for each pH value.

FIG. 14: illustrates voltage dependence of the mean fast positive current calculated between 30 and 50 μs (filled squares) and mean channel current calculated between 2 and 4 ms (empty circles) generated by VcChR1 expressed in HEK293 cells upon laser flash excitation. Data points are mean values±SEM of experiments in 3 cells.

FIG. 15: Deconvolution of CaChR1 M intermediate concentration changes into 5 kinetics components (as depicted in the table of FIG. 15 τ1-τ5). The first 3 components define accumulation of the intermediate, whereas the last two components define its disappearance.

FIG. 16: illustrates, inset, a typical photoinduced electrical signal recorded from a cell suspension of the marine alga P. subcordiformis consisting of a photoreceptor current followed by a regenerative response. Main panel, the action spectrum of photoreceptor currents in P. subcordiformis, which shows a contribution of two photoreceptor pigments. rel. u., relative unit.

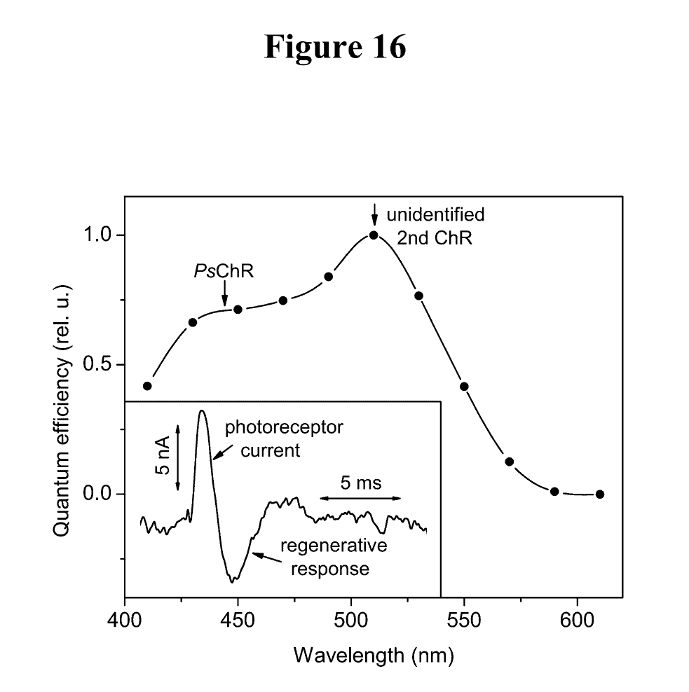

FIG. 17 (A-D): illustrates photocurrents generated by PsChR (A) and CrChR2 (B) expressed in HEK293 cells in response to a light pulse of saturating intensity (440 and 470 nm, respectively). The duration of the pulse is shown schematically on top. C and D, the mean amplitudes of peak and plateau currents (C) and the degree of current inactivation (D) for PsChR (n=27 cells) and CrChR2 (n=20 cells).

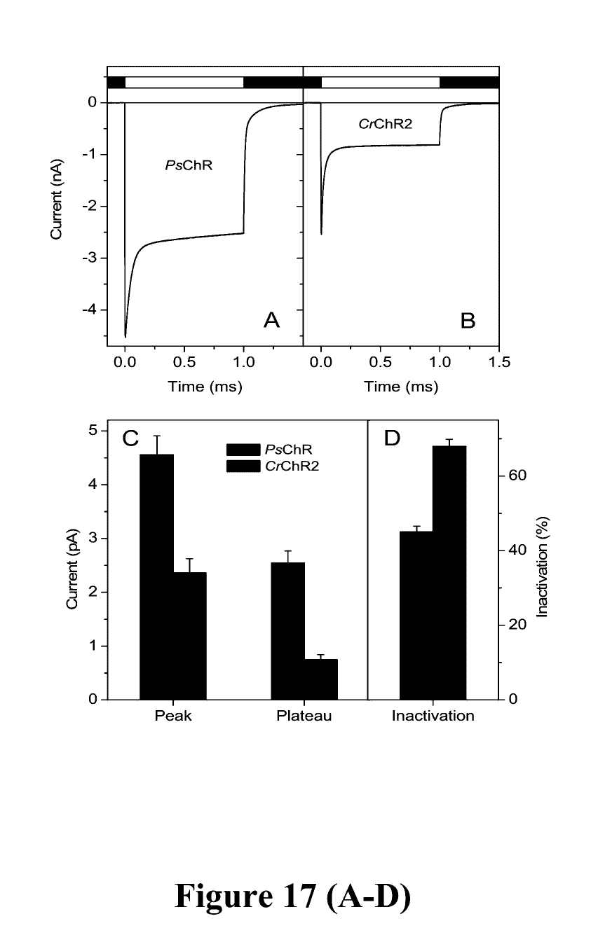

FIG. 18 (A-D): illustrates in panel A, whole cell current noise generated by PsChR (top) and CrChR2 (bottom) in the dark and under continuous illumination. In panel B, noise power spectra calculated for the dark (black line) and light (red line) conditions of currents generated by PsChR. The spectra were smoothed by five-point adjacent averaging for presentation purposes. Other details are under “Experimental Procedures.” In panel C, the difference spectrum (light minus dark) calculated from the spectra shown in B (black line) and its single Lorentzian fit (red line). In panel D, plateau currents measured after 1-s illumination (left axis; same as in FIG. 2C) and unitary conductances (right axis) of PsChR and CrChR2. The data are the mean values calculated from 12 individual current traces for PsChR and 8 traces for CrChR2. In some embodiment the absolute values of unitary conductance for both pigments determined by such a method may be underestimated because the plateau current generated by ChRs under prolonged illumination may be determined by a third, inactivated state of the channel, which is not taken into account by the model used. However in some further embodiments as presented herein the relative values indicate that PsChR exhibits ˜3-fold greater unitary conductance than CrChR2.

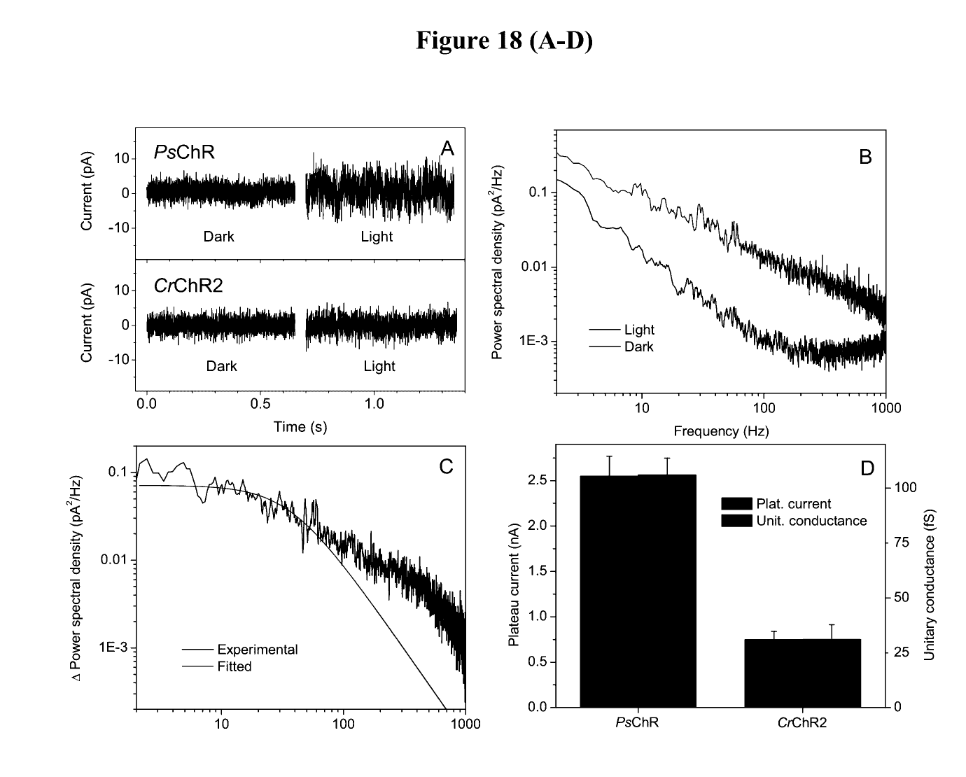

FIG. 19(A-B): illustrates, in panel A, photocurrents generated by PsChR expressed in an HEK293 cell in response to a pair of 1-s light pulses (440 nm, saturating intensity) delivered with a 1-s dark interval. Current was normalized to the peak value of the first signal. The illumination protocol is shown schematically on top. In panel B, the time course of the recovery of the transient component of the signal measured with PsChR (black squares) or CrChR2 (open circles). Recovery was calculated as the ratio of the differences between the peak and the plateau values measured in response to the second and the first pulse. Recovery percentage is plotted against the time interval between the pulses. The data are the mean values of measurements in three to five cells.

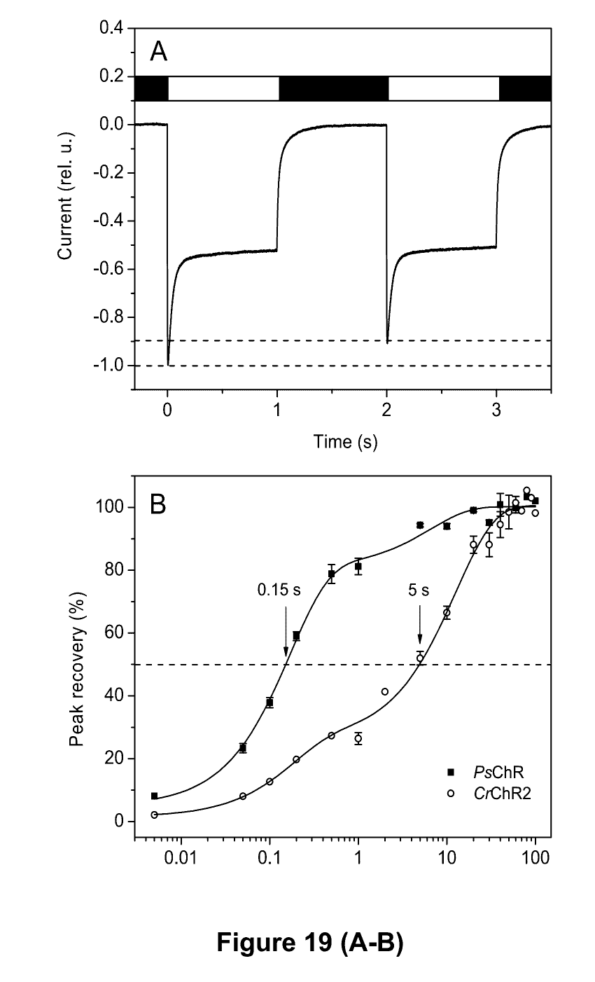

FIG. 20 (A-B): illustrates in panel A photocurrents generated by PsChR in the same HEK293 cell at 150 and at 1.5 mM Na+ and 148.5 mM nonpermeable N-methyl-D-glucamine in the bath (1-s light stimulus, 440 nm). In panel B, shifts of Vᵣ measured for plateau currents after a decrease in the bath Na+ concentration from 150 to 1.5 mM (left axis, solid bars), or an increase in the bath pH from 7.4 to 9 (right axis, hatched bars). The data points are the mean values obtained from three cells. channel. In contrast, no such inhibition was observed in PsChR:

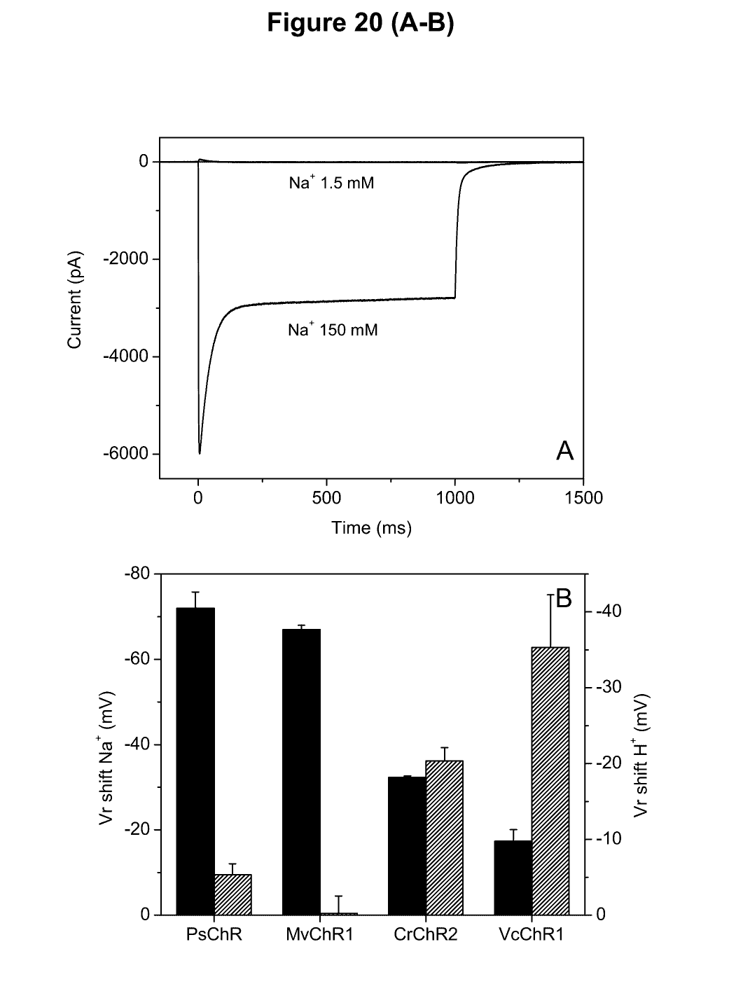

FIG. 21 (A-C): illustrates action spectra of channel currents generated in HEK293 cells by wild-type PsChR and its mutants at the bath pH 7.4 (A), wild-type CrChR2 and its mutants at the bath pH 7.4 (B), and wild-type PsChR at the bath pH indicated in the legend (C). The data points are the mean values of three to five measurements. rel. u., relative unit.

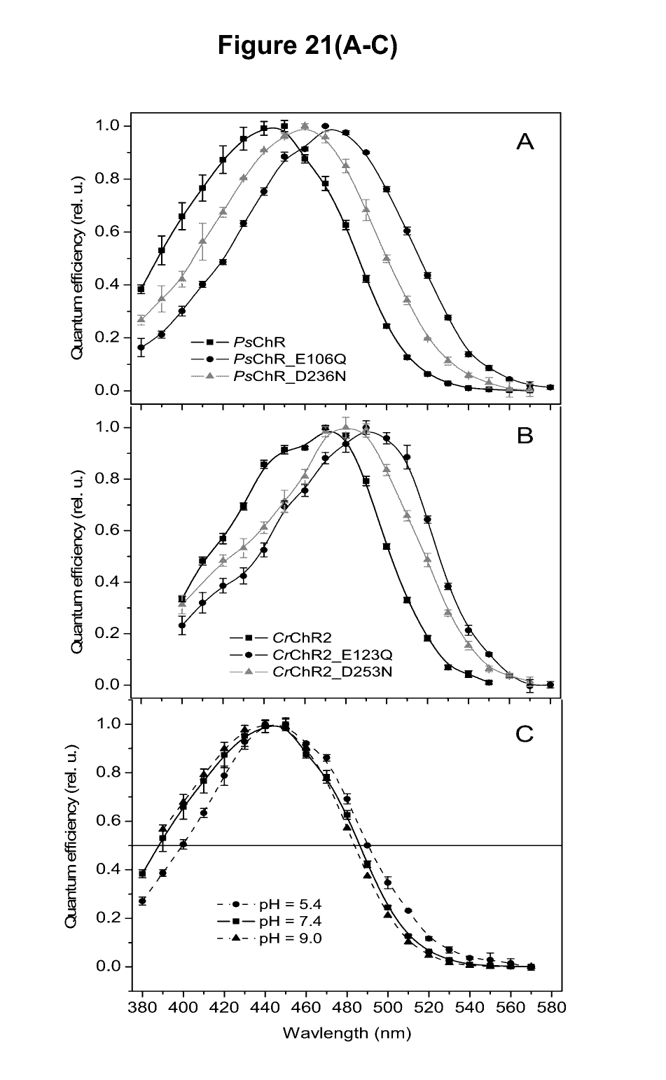

FIG. 22 (A-C): illustrates in panel A, the absorption spectrum of purified PsChR solubilized in detergent or reconstituted in nanodiscs. In panel B, pH titration of the absorption maximum of detergent-purified PsChR. Experimental data were fit with a sum of three Henderson-Hasselbalch components (f(pH)=AX(1−10(pH−pKa))−1+C). The pK values and amplitudes of the transitions denoted by A are derived from curve fitting. C, inset, the difference spectrum between dark- and light-adapted purified PsChR. Main panel, the time course of the spectral transition during dark adaptation determined as the dependence of the amplitude of the difference spectrum (as shown by the arrow in the inset) on time fit with an exponential function. rel. u., relative unit.

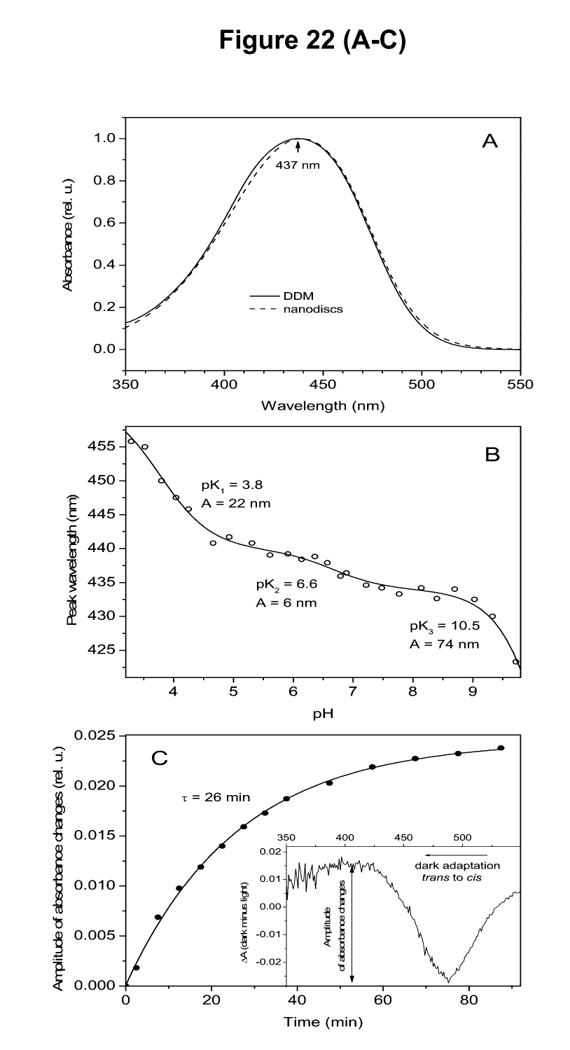

FIG. 23 (A-C): illustrates in panel A, the spectra of absorbance changes induced by laser excitation (532 nm, 6 ns) in detergent-purified PsChR. In panel B, the kinetics of the M-like intermediate in detergent-purified PsChR. The smooth line shows a computer fit to the data. In panel C, superposition of the kinetics of the red-shifted intermediate in the photocycle of detergent-purified PsChR and the kinetics of the laser flash-induced photocurrent generated by PsChR in a HEK293 cell the dependence of peak (squares) and plateau (circles) amplitudes on the stimulus intensity for currents generated by CrChR1 from C. raudensis. Data points are the mean normalized values±SEM (n=3).

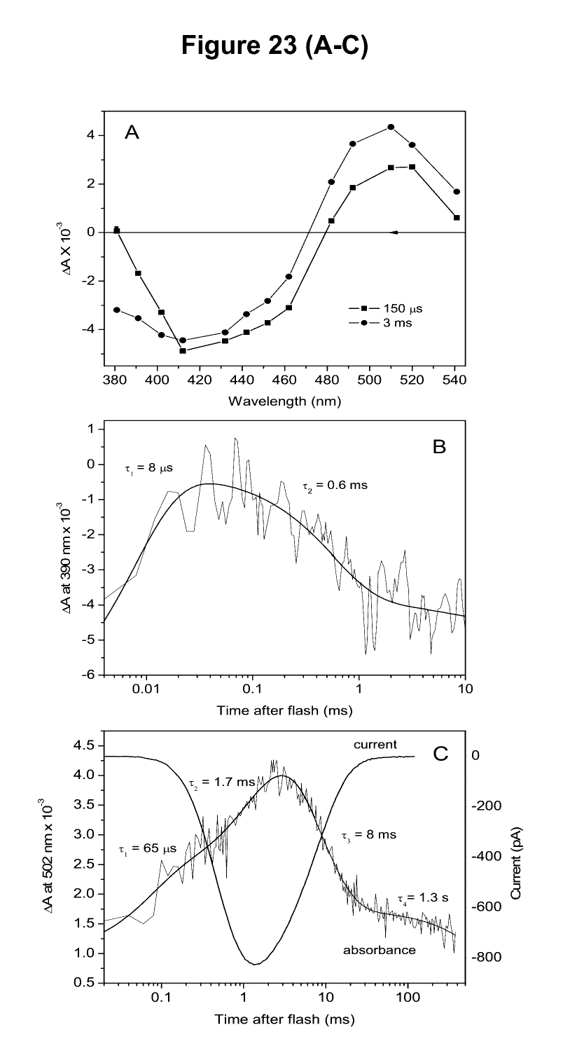

FIG. 24 (A-C): illustrates in panel A, typical voltage traces showing membrane depolarization and spikes recorded from a current-clamped PsChR-expressing pyramidal neuron in response to stimulation with brief light pulses (440 nm, 5 ms, 50 Hz). The times of light pulses are indicated by the bar at the bottom of the graph. In panels B and C, the normalized rate of the light-induced membrane depolarization (squares, left axis) and the probability of spike generation (bars, right axis) in cultured hippocampal neurons that express PsChR (B) or CrChR2 (C). Trains of 20 light pulses (5-ms duration) were applied at a frequency of 1 Hz. The excitation wavelength was 440 nm for PsChR and 470 nm for CrChR2, and the light intensity was close to the threshold for spike generation (i.e., spikes were evoked by less than 100% of pulses). The spiking probability was calculated for each successive group of five pulses. The data are mean values recorded from 16 experiments.

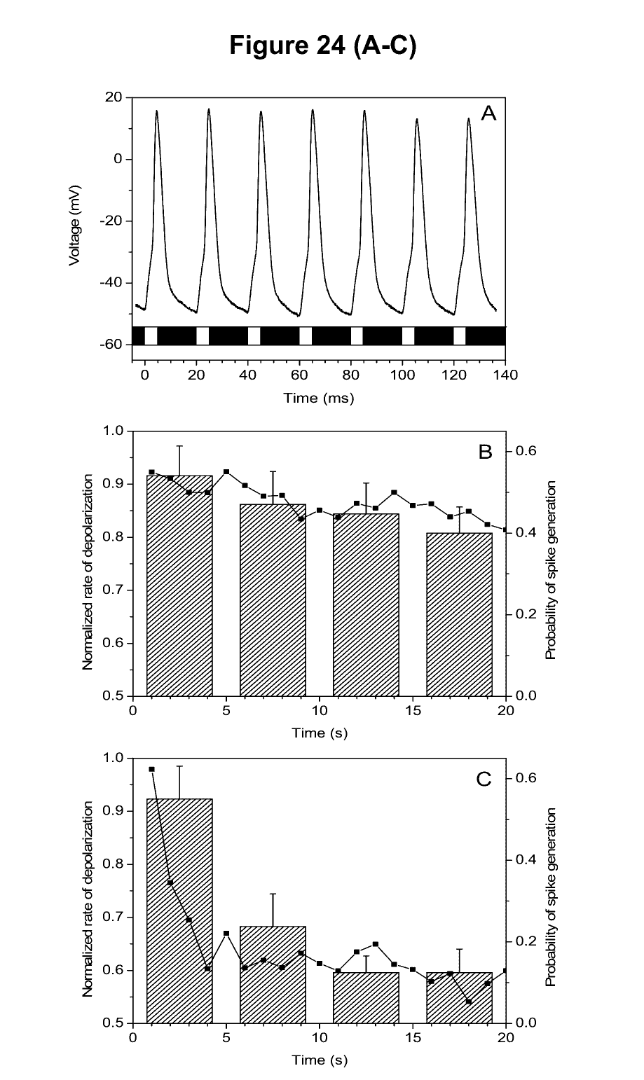

FIG. 25: illustrates an amino acid residue alignment of rhodopsin domains of known channelrhodopsins. Genbank accession numbers are given in brackets. PsChR, Platymonas (Tetraselmis) subcordiformis channelrhodopsin (JX983143) (SEQ ID No.1); CrChR1, Chlamydomonas reinhardtii channelrhodopsin 1, aka Chlamydomonas sensory rhodopsin A (AF508965) (SEQ ID No. 10); VcChR1, Volvox carteri channelrhodopsin 1 (EU622855) (SEQ ID No. 16); CaChR1, Chlamydomonas (Chloromonas) augustae channelrhodopsin 1 (JN596951) (SEQ ID No.5); CyChR1, Chlamydomonas yellowstonensis channelrhodopsin 1 (JN596948) (SEQ ID No. 8); HpChR1, Haematococcus pluvialis channelrhodopsin 1 (JN596950) (SEQ ID No. 15); DsChR1, Dunaliella salina channelrhodopsin 1 (JQ241364) (SEQ ID No. 4); PstChR1, Pleodorina starrii channelrhodopsin 1 (JQ249903) (SEQ ID No. 18); CrChR2, Chlamydomonas reinhardtii channelrhodopsin 2, aka Chlamydomonas sensory rhodopsin B (AF508966) (SEQ ID No. 7); VcChR2, Volvox carteri channelrhodopsin 2 (ABZ90903) (SEQ ID No. 17); CraChR2, Chlamydomonas raudensis channelrhodopsin 2 (JN596949) (SEQ ID No. 9); PstChR2, Pleodorina starrii channelrhodopsin 2 (JQ249904) (SEQ ID No. 19); MChR1, Mesostigma viride channelrhodopsin 1 (JF922293) (SEQ ID No. 14); and Pyramimonas gelidicola channelrhodopsin 1 (JQ241366) (SEQ ID No. 20).

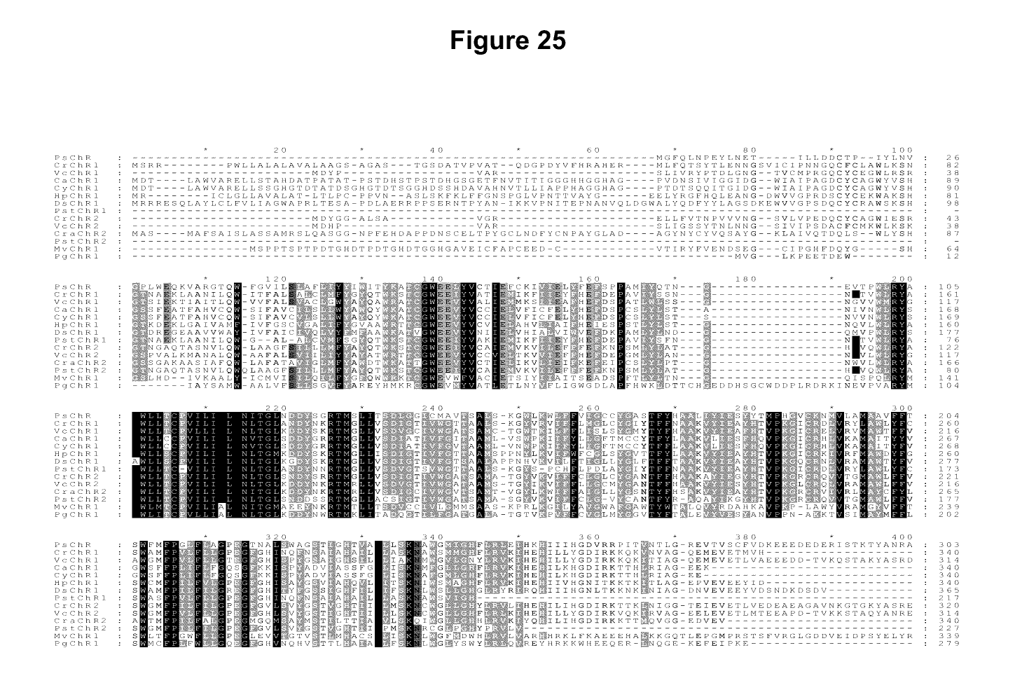

Conserved Glu residues in helix B are highlighted light green; a conserved Lys residue in helix B is highlighted yellow; homologs of the protonated Schiff base counterions Asp85 and Asp212 in bacteriorhodopsin are highlighted red; a His residue in the position of the Schiff base proton donor Asp96 in BR is highlighted blue; a homolog of Glu87 of CrChR1, responsible for its pH-dependent color tuning and fast photocurrent inactivation, is highlighted orange; Tyr and Asn in the position of Tyr226/Asn187 (in CrChR1/CrChR2, respectively), identified as one of the molecular determinants of differences in spectra, desensitization, and current kinetics, are highlighted as light blue and pink, respectively; homologs of Ser63 and Asn258 implicated in control of ion selectivity in CrChR2 are highlighted in olive; a homolog of Ser136 implicated in regulation of the size of the channel pore in CrChR2 is highlighted dark green; a homolog of Lys154 that contributes to a vestibule on the extracellular side of the channel pore in C1 C2 is highlighted purple.

### DESCRIPTION OF THE SEQUENCE LISTING

The Sequence Listing shows the amino acid sequences of a highly efficient blue-shifted rhodopsin domain (PsChR, SEQ ID NO: 1) derived from the channelrhodopsin of Platymonas (Tetraselmis) subcordiformis (a cDNA nucleic acid sequence shown in SEQ ID NO: 2 and the amino acid in SEQ ID NO: 3 which results from translation of this cDNA). In some embodiments all sequences listed herein are derived from cDNA. The rhodopsin domains of the channelrhodopsins from Dunaliella salina (DsChR1, SEQ ID NO: 4); Chlamydomonas augustae (CaChR1, SEQ ID NO: 5); Mesostigma viride (MChR1, SEQ ID NO: 6); Chlamydomonas reinhardtii (CrChR2, SEQ ID NO: 7); and Chlamydomonas yellowstonensis (CyChR1, SEQ ID NO: 8) are also provided. Further provided are the entire channelrhodopsin amino acid sequences derived from cDNA for Chlamydomonas raudensis (CraChR2, SEQ ID NO: 9); Chlamydomonas reinhardtii (CrChR1, SEQ ID NO: 10); Chlamydomonas reinhardtii (CrChR2, SEQ ID NO: 11); Chlamydomonas yellowstonensis (CyChR1, SEQ ID NO: 12); Chlamydomonas augustae (CaChR1, SEQ ID NO: 13); Mesostigma viride (MChR1, SEQ ID NO: 14); Haematococcus pluvialis (HpChR1, SEQ ID NO: 15); Volvox carteri (VcChR1, SEQ ID NO: 16); Volvox carteri (VcChR2, SEQ ID NO: 17); Pleodorina starrii (PstChR1, SEQ ID NO: 18); Pleodorina starrii (PstChR2, SEQ ID NO: 19); and Pyramimonas gelidicola (PgChR1, SEQ ID NO: 20). SEQ ID NOS: 21 and 23 are forward PCR primers, and SEQ ID NOS: 22 and 24 are reverse PCR primers.

### DETAILED DESCRIPTION

### Definitions

In this disclosure, the use of the singular includes the plural, the word “a” or “an” means “at least one”, and the use of “or” means “and/or”, unless specifically stated otherwise. Furthermore, the use of the term “including”, as well as other forms, such as “includes” and “included”, is not limiting. Also, terms such as “element” or “component” encompass both elements and components comprising one unit and elements or components that comprise more than one unit unless specifically stated otherwise.

As used herein, the term “about,” when used in conjunction with a percentage or other numerical amount, means plus or minus 10% of that percentage or other numerical amount. For example, the term “about 80%,” would encompass 80% plus or minus 8%.

The section headings used herein are for organizational purposes only and are not to be construed as limiting the subject matter described. All documents, or portions of documents, cited in this application, including, but not limited to, patents, patent applications, articles, books, and treatises, are hereby expressly incorporated herein by reference in their entirety for any purpose. In the event that one or more of the incorporated literature and similar materials defines a term in a manner that contradicts the definition of that term in this application, this application controls

As used herein, and unless otherwise indicated, the term neuron mediated disorders for which the present methods and compositions may be used include, but are not limited to, neuronal dysfunctions, disorders of the brain, the central nervous system, the peripheral nervous system, neurological conditions, disorders of memory and leaning disorders, cardiac arrhythmias, Parkinson's disease, ocular disorders, spinal cord injury among others.

As used herein, and unless otherwise indicated, the term ocular disorders for which the present methods and compositions may be used to improve one or more parameters of vision include, but are not limited to, developmental abnormalities that affect both anterior and posterior segments of the eye. Anterior segment disorders include, but are not limited to, glaucoma, cataracts, corneal dystrophy, keratoconus. Posterior segment disorders include, but are not limited to, blinding disorders caused by photoreceptor malfunction and/or death caused by retinal dystrophies and degenerations. Retinal disorders include congenital stationary night blindness, age-related macular degeneration, congenital cone dystrophies, and a large group of retinitis-pigmentosa (RP)-related disorders.

As used herein, and unless otherwise indicated, the terms “treat,” “treating,” “treatment” and “therapy” contemplate an action that occurs while a patient is suffering from an ocular disorder that reduces the severity of one or more symptoms or effects of an ocular disorder. Where the context allows, the terms “treat,” “treating,” and “treatment” also refers to actions taken toward ensuring that individuals at increased risk of a neuron mediated disorder or ocular disorders, are able to receive appropriate surgical and/or other medical intervention prior to onset of a neuron mediated disorder or ocular disorder. As used herein, and unless otherwise indicated, the terms “prevent,” “preventing,” and “prevention” contemplate an action that occurs before a patient begins to suffer from a neuron mediated disorder or ocular disorder, that delays the onset of, and/or inhibits or reduces the severity of a neuron mediated disorder or ocular disorder.

As used herein, and unless otherwise indicated, the terms “manage,” “managing,” and “management” encompass preventing, delaying, or reducing the severity of a recurrence of an ocular disorder in a patient who has already suffered from such a disease, disorder or condition. The terms encompass modulating the threshold, development, and/or duration of the ocular disorder or changing how a patient responds to a neuron mediated disorder or ocular disorder.

As used herein, and unless otherwise specified, a “therapeutically effective amount” of a compound is an amount sufficient to provide any therapeutic benefit in the treatment or management of a neuron mediated disorder or ocular disorder, or to delay or minimize one or more symptoms associated with a neuron mediated disorder or ocular disorder. A therapeutically effective amount of a compound means an amount of the compound, alone or in combination with one or more other therapies and/or therapeutic agents that provide any therapeutic benefit in the treatment or management of a neuron mediated disorder or ocular disorder. The term “therapeutically effective amount” can encompass an amount that alleviates a neuron mediated disorder or ocular disorder, improves or reduces an ocular disorder, improves overall therapy, or enhances the therapeutic efficacy of another therapeutic agent.

As used herein, and unless otherwise specified, a “prophylactically effective amount” of a compound is an amount sufficient to prevent or delay the onset of a neuron mediated disorder or ocular disorder, or one or more symptoms associated with an ocular disorder or prevent or delay its recurrence. A prophylactically effective amount of a compound means an amount of the compound, alone or in combination with one or more other treatment and/or prophylactic agent that provides a prophylactic benefit in the prevention of a neuron mediated disorder or ocular disorder. The term “prophylactically effective amount” can encompass an amount that prevents a neuron mediated disorder or ocular disorder, improves overall prophylaxis, or enhances the prophylactic efficacy of another prophylactic agent. The “prophylactically effective amount” can be prescribed prior to, for example, the development of a neuron mediated disorder or ocular disorder.

As used herein, “patient” or “subject” includes mammalian organisms which are capable of suffering from an ocular disorder as described herein, such as human and non-human mammals, for example, but not limited to, rodents, mice, rats, non-human primates, companion animals such as dogs and cats as well as livestock, e.g., sheep, cow, horse, etc.

As used herein, the term “conservative substitution” generally refers to amino acid replacements that preserve the structure and functional properties of a protein or polypeptide. Such functionally equivalent (conservative substitution) peptide amino acid sequences include, but are not limited to, additions or substitutions of amino acid residues within the amino acid sequences encoded by a nucleotide sequence that result in a silent change, thus producing a functionally equivalent gene product. Conservative amino acid substitutions may be made on the basis of similarity in polarity, charge, solubility, hydrophobicity, hydrophilicity, and/or the amphipathic nature of the residues involved. For example: nonpolar (hydrophobic) amino acids include alanine, leucine, isoleucine, valine, proline, phenylalanine, tryptophan, and methionine; polar neutral amino acids include glycine, serine, threonine, cysteine, tyrosine, asparagine, and glutamine; positively charged (basic) amino acids include arginine, lysine, and histidine; and negatively charged (acidic) amino acids include aspartic acid and glutamic acid.

As used herein, a “redshift” is a shift to longer wavelength. In contrast a “blueshift” would be a shift to shorter wavelength. These terms apply to both light-emitting and light-absorbing objects.

Light control of motility behavior (phototaxis and photophobic responses) in green flagellate algae is mediated by sensory rhodopsins homologous to phototaxis receptors and light-driven ion transporters in prokaryotic organisms. In the phototaxis process, excitation of the algal sensory rhodopsins leads to generation of transmembrane photoreceptor currents. When expressed in animal cells, the algal phototaxis receptors function as light-gated cation channels, which have earned them the name “channelrhodopsins”. Channelrhodopsins have become useful molecular tools for light control of cellular activity.

Described herein in some embodiments are compositions and methods for use in generating and obtaining a blue-shifted channelrhodopsin from algae that is superior to those currently available. Channelrhodopsins are phototaxis receptors that function as light-gated cation channels when transfected into animal cells, are used for photoactivation of neuron firing. As used herein the term “channelrhodopsin” describes retinylidene proteins (rhodopsins) that function as light-gated ion channels.

Also used herein, the phrase “rhodopsin domain” refers to the “rhodopsin fold”, a 7-transmembrane-helix (7TM) structure characteristic of rhodopsins.

Generally, for optogenetic tools long-wavelength absorption is preferable to minimize scattering of light by biological tissues and its absorption by hemoglobin. However, the blue-shifted spectrum is ideally suited for combinatorial applications with long wavelength-absorbing rhodopsin pumps or fluorescent indicators.

As used herein, the channelopsin is the apoprotein, while channelrhodopsin is the protein and retinal. The amino acid sequence identified in SEQ ID NOS: 1, 4 and 5-8, define the opsin, but this is also the sequence of the rhodopsin. By screening phototaxis receptor currents among several algal species, a highly efficient blue-shifted channelrhodopsin with rapid kinetics was identified and characterized. In some embodiments, the disclosed methods provide a technology that facilities the identification and characterization of particularly useful channelrhodopsins from algae (such as but not limited to: Platymonas (Tetraselmis), Mesostigma viride, Chlamydomonas augustae, Chlamydomonas yellowstonensis, Chlamydomonas reinhardtii, Acetabularia, Ulva, Pyramimonas).

Some embodiments provided herein are amino acid and nucleic acid sequences of functional domains of novel channelrhodopsins that are also functionally characterized. One such channelrhodopsin has been determined to have blue-shifted absorption maxima and a functional rhodopsin domain of the channelrhodopsin was cloned and identified as PsChR (SEQ ID NO: 1) which was derived from channelrhodopsin of Platymonas (Tetraselmis) subcordiformis (SEQ ID NOS: 2 and 3, EMBL entry JX983143). Also provided in some embodiments is the use and composition of this novel channelrhodopsin domain, identified as PsChR1 (SEQ ID NO: 1).

In some embodiments, are conserved variants of PsChR or a peptide fragment thereof. A “conservative” amino acid substitution refers to the substitution of an amino acid in a polypeptide with another amino acid having similar properties, such as size or charge. In certain embodiments, a polypeptide comprising a conservative amino acid substitution maintains at least one activity of the unsubstituted polypeptide. A conservative amino acid substitution may encompass non-naturally occurring amino acid residues which are typically incorporated by chemical peptide synthesis rather than by synthesis in biological systems. These include, but are not limited to, peptidomimetics and other reversed or inverted forms of amino acid moieties.

Naturally occurring residues may be divided into classes based on common side chain properties: hydrophobic (Met, Ala, Val, Leu, Ile); neutral hydrophilic (Cys, Ser, Thr, Asn, Gln); acidic (Asp, Glu); basic (His, Lys, Arg); residues that influence chain orientation (Gly, Pro); and aromatic(Trp,Tyr, Phe). For example, non-conservative substitutions may involve the exchange of a member of one of these classes for a member from another class.

In making substitutions, according to certain embodiments, the hydropathic index of amino acids may be considered. Each amino acid has been assigned a hydropathic index on the basis of its hydrophobicity and charge characteristics. They are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein, in certain instances, is understood in the art (Kyte et al., J. Mol. Biol., 157:105-131 (1982)). It is known that in certain instances, certain amino acids may be substituted for other amino acids having a similar hydropathic index or score and still retain a similar biological activity. In making changes based upon the hydropathic index, in certain embodiments, the substitution of amino acids whose hydropathic indices are within ±2 is included. In certain embodiments, those which are within ±1 are included, and in certain embodiments, those within ±0.5 are included.

Substitution of like amino acids can be made effectively on the basis of hydrophilicity, particularly where the biologically functional protein or peptide thereby created is intended for use in immunological embodiments, as in the present case. In certain embodiments, the greatest local average hydrophilicity of a protein, as governed by the hydrophilicity of its adjacent amino acids, correlates with its immunogenicity and antigenicity, i.e., with a biological property of the protein.

The following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5) and tryptophan (−3.4). In making changes based upon similar hydrophilicity values, in certain embodiments, the substitution of amino acids whose hydrophilicity values are within ±2 is included, in certain embodiments, those which are within ±1 are included, and in certain embodiments, those within ±0.5 are included.

A skilled artisan will be able to determine suitable variants of a polypeptide as set forth herein using well-known techniques. In certain embodiments, one skilled in the art may identify suitable areas of the molecule that may be changed without destroying activity by targeting regions not believed to be important for activity. In certain embodiments, one can identify residues and portions of the molecules that are conserved among similar polypeptides.

In certain embodiments, even areas that may be important for biological activity or for structure may be subject to conservative amino acid substitutions without destroying the biological activity or without adversely affecting the polypeptide structure.

Additionally, in certain embodiments, one skilled in the art can review structure-function studies identifying residues in similar polypeptides that are important for activity or structure. In view of such a comparison, in certain embodiments, one can predict the importance of amino acid residues in a protein that correspond to amino acid residues which are important for activity or structure in similar proteins. In certain embodiments, one skilled in the art may opt for chemically similar amino acid substitutions for such predicted important amino acid residues.

In certain embodiments, one skilled in the art can also analyze the three-dimensional structure and amino acid sequence in relation to that structure in similar polypeptides. In certain embodiments, in view of such information, one skilled in the art may predict the alignment of amino acid residues of a polypeptide with respect to its three dimensional structure. In certain embodiments, one skilled in the art may choose not to make radical changes to amino acid residues predicted to be on the surface of the protein, since such residues may be involved in important interactions with other molecules.

Moreover, in certain embodiments, one skilled in the art may generate test variants containing a single amino acid substitution at each desired amino acid residue. In certain embodiments, the variants can then be screened using activity assays known to those skilled in the art. In certain embodiments, such variants could be used to gather information about suitable variants. For example, in certain embodiments, if one discovered that a change to a particular amino acid residue resulted in destroyed, undesirably reduced, or unsuitable activity, variants with such a change may be avoided. In other words, in certain embodiments, based on information gathered from such routine experiments, one skilled in the art can readily determine the amino acids where further substitutions should be avoided either alone or in combination with other mutations.

A number of scientific publications have been devoted to the prediction of secondary structure. See, e.g., Moult J., Curr. Op. in Biotech., 7(4):422-427 (1996), Chou et al., Biochemistry, 13(2):222-245 (1974); Chou et al., Biochemistry, 113(2):211-222 (1974); Chou et al., Adv. Enzymol. Relat. Areas Mol. Biol., 47:45-148 (1978); Chou et al., Ann. Rev. Biochem., 47:251-276 and Chou et al., Biophys. J., 26:367-384 (1979). Moreover, computer programs are currently available to assist with predicting secondary structure. One method of predicting secondary structure is based upon homology modeling. For example, two polypeptides or proteins which have a sequence identity of greater than 30%, or similarity greater than 40% often have similar structural topologies. The growth of the protein structural database (PDB) has provided enhanced predictability of secondary structure, including the potential number of folds within a polypeptide's structure. See, e.g., Holm et al., Nucl. Acid. Res., 27(1):244-247 (1999). It has been suggested (Brenner et al., Curr. Op. Struct. Biol., 7(3):369-376 (1997)) that there are a limited number of folds in a given polypeptide or protein and that once a critical number of structures have been resolved, structural prediction will become dramatically more accurate.

Additional methods of predicting secondary structure include “threading” (see, e.g., Jones, D., Curr. Opin. Struct. Biol., 7(3):377-87 (1997); Sippl et al., Structure, 4(1):15-19 (1996)), “profile analysis” (see, e.g., Bowie et al., Science, 253:164-170 (1991); Gribskov et al., Meth. Enzym., 183:146-159 (1990); Gribskov et al., Proc. Nat. Acad. Sci., 84(13):4355-4358 (1987)), and “evolutionary linkage” (see, e.g., Holm et al., Nucl. Acid. Res., 27(1):244-247 (1999), and Brenner et al., Curr. Op. Struct. Biol., 7(3):369-376 (1997)).

In certain embodiments, a variant of the reference channelrhodopsin or rhodopsin domain (PsChR) includes a glycosylation variant wherein the number and/or type of glycosylation sites have been altered relative to the amino acid sequence of the reference channelrhodopsin or rhodopsin domain (PsChR). In certain embodiments, a variant of a polypeptide comprises a greater or a lesser number of N-linked glycosylation sites relative to a native polypeptide. An N-linked glycosylation site is characterized by the sequence: Asn-X-Ser or Asn-X-Thr, wherein the amino acid residue designated as X may be any amino acid residue except proline. The substitution of amino acid residues to create this sequence provides a potential new site for the addition of an N-linked carbohydrate chain. Alternatively, substitutions which eliminate this sequence will remove an existing N-linked carbohydrate chain. In certain embodiments, a rearrangement of N-linked carbohydrate chains is provided, wherein one or more N-linked glycosylation sites (typically those that are naturally occurring) are eliminated and one or more new N-linked sites are created. Exemplary variants include cysteine variants wherein one or more cysteine residues are deleted from or substituted for another amino acid (e.g., serine) relative to the amino acid sequence of the reference channelrhodopsin or rhodopsin domain (PsChR). In certain embodiments, cysteine variants may be useful when polypeptides and proteins must be refolded into a biologically active conformation such as after the isolation of insoluble inclusion bodies. In certain embodiments, cysteine variants have fewer cysteine residues than the native polypeptide. In certain embodiments, cysteine variants have an even number of cysteine residues to minimize interactions resulting from unpaired cysteines.

According to certain embodiments, amino acid substitutions are those which: (1) reduce susceptibility to proteolysis, (2) reduce susceptibility to oxidation, (3) alter binding affinity for forming protein complexes, (4) alter binding affinities, and/or (4) confer or modify other physiochemical or functional properties on such polypeptides. According to certain embodiments, single or multiple amino acid substitutions (in certain embodiments, conservative amino acid substitutions) may be made in a naturally-occurring sequence (in certain embodiments, in the portion of the polypeptide outside the domain(s) forming intermolecular contacts). In certain embodiments, a conservative amino acid substitution typically may not substantially change the structural characteristics of the reference sequence (e.g., in certain embodiments, a replacement amino acid should not tend to break a helix that occurs in the reference sequence, or disrupt other types of secondary structure that characterizes the reference sequence).

Examples of certain art-recognized polypeptide secondary and tertiary structures are described, for example, in Proteins, Structures and Molecular Principles (Creighton, Ed., W. H. Freeman and Company, New York (1984)); Introduction to Protein Structure (C. Branden and J. Tooze, eds., Garland Publishing, New York, N.Y. (1991)); and Thornton et at. Nature 354:105 (1991).

In other embodiments, are methods and compositions that provide a channelrhodopsin with improved properties and characteristics that enhance the application of the compositions in, among other things, optogenetic techniques. These embodiments provide greater unitary conductance, sodium specificity, or the enhancement of the short-wavelength sensitivity, by inducing a blueshift in absorption maxima. Matching the spectral properties of PsChR (SEQ ID NO: 1) with the action spectrum of photoreceptor currents in P. subcordiformis clearly indicates that it is the blue-shifted member of the pair, and consequently can be classified as PsChR2, despite it being the only so far identified ChR in this alga. PsChR absorption is the most blue-shifted of all known ChRs. Generally, for optogenetic tools long-wavelength absorption is preferable to minimize scattering of light by biological tissues and its absorption by hemoglobin. However, the blue-shifted spectrum of PsChR (SEQ ID NO: 1) is ideal for combinatorial applications with long wavelength-absorbing rhodopsin pumps or fluorescent indicators.

Optogenetic techniques involve the introduction of light-activated channels and enzymes that allow manipulation of neural activity and control of neuronal function. Thus, in some embodiments, the disclosed methods and compositions can be introduced into cells and facilitate the manipulation of the cells activity and function. See, for example, US publication 20130090454 of U.S. application Ser. No. 13/622,809, as well as, Mattis, J., Tye, K. M., Ferenczi, E. A., Ramakrishnan, C., O'Shea, D. J., Prakash, R., Gunaydin, L. A., Hyun, M., Fenno, L. E., Gradinaru, V., Yizhar, O., and Deisseroth, K. (2012) Principles for applying optogenetic tools derived from direct comparative analysis of microbial opsins. Nat. Methods 9, 159-172; and Zhang, F., Vierock, J., Yizhar, O., Fenno, L. E., Tsunoda, S., Kianianmo-meni, A., Prigge, M., Berndt, A., Cushman, J., Polle, J., Magnuson, J., Hege-mann, P., and Deisseroth, K. (2011) The microbial opsin family of optogenetic tools. Cell 147, 1446-1457).

Optogenetic techniques, and thus the disclosed methods and compositions, can be used to characterize the functions of complex neural circuits and information processing in the normal brain and during various neurological conditions; functionally map the cerebral cortex; characterize and manipulate the process of learning and memory; characterize and manipulate the process of synaptic transmission and plasticity; provide light-controlled induction of gene expression; provide optical control of cell motility and other activities.

Clinical applications of the disclosed methods and compositions include (but are not limited to) optogenetic approaches to therapy such as: restoration of vision by introduction of channelrhodopsins in post-receptor neurons in the retina for ocular disorder gene-therapy treatment of age-dependent macular degeneration, diabetic retinopathy, and retinitis pigmentosa, as well as other conditions which result in loss of photoreceptor cells; control of cardiac function by using channelrhodopsins incorporated into excitable cardiac muscle cells in the atrioventricular bundle (bundle of His) to control heart beat rhythm rather than an electrical pacemaker device; restoration of dopamine-related movement dysfunction in Parkinsonian patients; amelioration of depression; recovery of breathing after spinal cord injury; provide noninvasive control of stem cell differentiation and assess specific contributions of transplanted cells to tissue and network function.

Currents generated by PsChR (SEQ ID NO: 1) in HEK293 cells show higher amplitude, smaller inactivation, and faster peak recovery than those generated by CrChR2, the molecule of choice in most optogenetic studies, whereas their kinetics is similarly fast. The higher current amplitude of PsChR is due to its higher unitary conductance, as estimated by stationary noise analysis. Therefore, in some embodiments a blue-shifted channelrhodopsin with higher amplitude, smaller inactivation, and faster peak recovery is provided as PsChR (SEQ ID NO: 1) which was derived from channelrhodopsin of Platymonas (Tetraselmis) subcordiformis and may be used to enhance optogenetic techniques and optogenetic approaches to therapy.

Channelrhodopsins, functional or active portions thereof, such as but not limited to the rhodopsin domain, and functional equivalents include, but are not limited to, naturally occurring versions of channelrhodopsin and those that are orthologs and homologs, and mutant versions of channelrhodopsin, whether naturally occurring or engineered (by site directed mutagenesis, gene shuffling, or directed evolution, as described in, for example, U.S. Pat. No. 5,837,458). Also included are the use of degenerate nucleic acid variants (due to the redundancy of the genetic code) of the disclosed an algae derived channelrhodopsin polynucleotide sequences.

In some embodiments, are isolated nucleic acid molecules comprising a nucleotide sequence that encodes a highly efficient, sodium specific, blue-shifted channelrhodopsin derived from algae. In some embodiments, the rhodopsin domain encodes the peptides whose sequence is described in SEQ ID NO: 1. In some embodiments, are isolated nucleic acid molecules comprising a nucleotide sequence that encodes the amino acid sequence shown in SEQ ID NO: 1 or SEQ ID NO: 3. In some embodiments, are expression vectors comprising a nucleic acid sequence that encodes the amino acid sequences of SEQ ID NO: 1 or SEQ ID NO: 3. In some embodiments, are host cells comprising a expression vector comprising a nucleic acid sequence that encodes the amino acid sequences of SEQ ID NO: 1 or SEQ ID NO: 3 or a nucleic acid sequence, fragment of portion thereof of the cDNA of SEQ ID NO:2.

In some embodiments, are isolated nucleic acid molecules comprising a nucleotide sequence that was derived from cDNA and encode the rhodopsin domain of a channelrhodopsin derived from algae. In some embodiments, the rhodopsin domain encodes the peptides whose sequence is described in SEQ ID NO: 1 or SEQ ID NO: 3. In some embodiments, are isolated nucleic acid molecules that were derived from cDNA that comprise a nucleotide sequence that encodes the amino acid sequence shown in SEQ ID NO: 1 or SEQ ID NO: 3. In some embodiments, are expression vectors comprising a nucleic acid sequence that encodes the amino acid sequences of SEQ ID NO: 1 or SEQ ID NO: 3. In some embodiments, are host cells comprising a recombinant expression vector comprising a nucleic acid sequence that was derived from cDNA and encode the amino acid sequences of SEQ ID NO: 1 or SEQ ID NO: 3.

In some embodiments, are isolated peptides comprising an amino acid sequence encoded by at least a portion of the cDNA derived nucleic acid sequences that encode the 7TM or rhodopsin domain of a highly efficient, sodium specific, blue-shifted channelrhodopsin derived from algae. In some embodiments, are isolated peptides comprising an amino acid sequence encoded by a cDNA derived nucleic acid sequence that encodes an amino acid sequence of a group consisting of SEQ ID NO: 1 or SEQ ID NO: 3.

In some embodiments, are isolated peptides comprising a contiguous sequence encoded by a cDNA derived nucleic acid sequence that encodes the rhodopsin domain of a highly efficient, sodium specific, blue-shifted channelrhodopsin derived from algae. In some embodiments, are isolated peptides comprising an amino acid sequence of SEQ ID NO: 1 or SEQ ID NO: 3, or fragment thereof. In some embodiments, are isolated peptides comprising an amino acid sequence encoded by at least a portion of a cDNA derived nucleic acid sequence of a group consisting of SEQ ID NO: 2 and which functions as a rhodopsin or channelrhodopsin.

In some embodiments, are isolated peptides comprising an amino acid sequence of SEQ ID NO: 1 or SEQ ID NO: 3 or a 7 TM domain /rhodopsin domain encoded by a cDNA derived nucleic acid sequence of SEQ ID NO: 2 and function as a channelrhodopsin.

In some embodiments, are isolated nucleic acid molecules comprising a nucleotide sequence that encodes the rhodopsin of a highly efficient, sodium specific, blue-shifted channelrhodopsin derived from algae. In some embodiments, the rhodopsin encodes a peptide whose sequence is described in a group consisting of SEQ ID NO: 1 or SEQ ID NO: 3. In some embodiments, are isolated nucleic acid molecules comprising a nucleotide sequence that encodes the amino acid sequence shown in SEQ ID NO: 1 or SEQ ID NO: 3. In some embodiments, are expression vectors comprising a nucleic acid sequence that encodes the amino acid sequences of SEQ ID NO: 1 or SEQ ID NO: 3. In some embodiments, are host cells comprising a expression vector comprising a nucleic acid sequence that encodes the amino acid sequences of SEQ ID NO: 1 or SEQ ID NO: 3. In some embodiments, are peptides comprising a sequence that encodes the rhodopsin domain of a highly efficient, sodium specific, blue-shifted channelrhodopsin derived from algae. In some embodiments, are isolated peptides comprising an amino acid sequence of a group consisting of SEQ ID NO: 1 or SEQ ID NO: 3. In some embodiments, are isolated peptides comprising a sequence that encodes the rhodopsin domain of channelrhodopsin derived from algae. In some embodiments, the isolated peptides comprise an amino acid sequence of SEQ ID NO: 1 or SEQ ID NO: 3, or fragments thereof.

In some embodiments, are isolated nucleic acid molecules wherein said nucleic acid molecule has a sequence is selected from the group consisting of SEQ ID NO: 2. In other embodiments, are expression vectors comprising a nucleic acid sequence selected from the group consisting of SEQ ID NO: 2 and those that encode the amino acid sequences of SEQ ID NO: 1 or SEQ ID NO: 3. In some embodiments, are host cells comprising a expression vector comprising a nucleic acid sequence selected from the group consisting of SEQ ID NO: 2 and those that encode the amino acid sequences of SEQ ID NO: 1 or SEQ ID NO: 3. In some embodiments, an isolated nucleic acid comprises a nucleotide sequence that encodes the t rhodopsin domain of a highly efficient, sodium specific, blue-shifted channelrhodopsin derived from algae. In some embodiments, the nucleotide sequence encodes at least 16 contiguous amino acids of SEQ ID NO: 1 or SEQ ID NO: 3. In some embodiments, the nucleotide sequence encodes at least 20 contiguous amino acids of SEQ ID NO: 1 or SEQ ID NO: 3. In some embodiments, the nucleotide sequence encodes at least 35 contiguous amino acids of SEQ ID NO: 1, or SEQ ID NO: 3. In some embodiments, the nucleotide sequence encodes at least 50 contiguous amino acids of SEQ ID NO:1 or SEQ ID NO: 3. In some embodiments, the nucleotide sequence encodes at least 75 contiguous amino acids of SEQ ID NO:1 or SEQ ID NO:3. In some embodiments, the nucleotide sequence encodes at least 33 contiguous amino acids of SEQ ID NO: 1 or SEQ ID NO: 3. In some embodiments, the nucleotide sequence encodes a peptide comprising any contiguous portion of SEQ ID NO: 1 or SEQ ID NO: 3.

In some embodiments, are isolated polypeptides that encode a rhodopsin domain of a highly efficient, sodium specific, blue-shifted channelrhodopsin derived from algae. In some embodiments, an isolated polypeptide comprising the amino acid sequence of SEQ ID NO: 1 or SEQ ID NO: 3. In some embodiments, the isolated polypeptide has at least 85% homology to the amino acid sequence of SEQ ID NO:1 or SEQ ID NO:3. In some embodiments, the isolated polypeptide has between 85%-100% homology to the amino acid sequence of SEQ ID NO: 1 or SEQ ID NO: 3.

In some embodiments, is a protein composition comprises a polypeptide having the amino acid sequence of SEQ ID NO: 1 or SEQ ID NO: 3.

In some embodiments, are isolated nucleic acids that comprise a nucleotide sequence that encodes the rhodopsin domain of a novel channelrhodopsin derived from Platymonas (Tetraselmis) subcordiformis. In some embodiments, the nucleotide sequence encodes at least 16 contiguous amino acids of SEQ ID NO: 1 or SEQ ID NO: 3. In some embodiments, the nucleotide sequence encodes at least 20 contiguous amino acids of SEQ ID NO: 1 or SEQ ID NO: 3. In some embodiments, the nucleotide sequence encodes at least 35 contiguous amino acids of SEQ ID NO: 1 or SEQ ID NO: 3. In some embodiments, the nucleotide sequence encodes at least 50 contiguous amino acids of SEQ ID NO: 1 or SEQ ID NO: 3. In some embodiments, the nucleotide sequence encodes at least 75 contiguous amino acids of SEQ ID NO: 1 or SEQ ID NO: 3. In some embodiments, the nucleotide sequence encodes at least 33 contiguous amino acids of SEQ ID NO: 1 or SEQ ID NO: 3. In some embodiments, the nucleotide sequence encodes a peptide comprising SEQ ID NO: 1 or SEQ ID NO: 3.

In some embodiments, an isolated polypeptide encodes the rhodopsin domain of a channelrhodopsin derived from C. raudensis. In some embodiments, an isolated polypeptide comprising the amino acid sequence of SEQ ID NO: 1 or SEQ ID NO: 3. In some embodiments, the isolated polypeptide has at least 85% homology to the amino acid sequence of SEQ ID NO: 1 or SEQ ID NO: 3. In some embodiments, a protein composition comprises a polypeptide having the amino acid sequence of SEQ ID NO: 1 or SEQ ID NO: 3.

In some embodiments, an isolated nucleic acid comprising a nucleotide sequence that encodes a functional domain of a channelrhodopsin of Platymonas (Tetraselmis) subcordiformi. In some embodiments are isolated nucleic acid that encodes at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 75, 100, 125, 150, 175, 200, 205, 210, 215, 220, 225, 228, 229, 230, 235, 240 250, 255, 260, 265, 270, 275, 280, 285, 290, 295, 300, 305, 310, 315, 320, 325, 350, 375, 400, 500, 600, 700, 800, 900 or more contiguous amino acids of SEQ ID NO: 1 or SEQ ID NO: 3 or fragments thereof. Further, in some embodiments, any range derivable between any of the above-described integers.

In other embodiments, the present invention provides for an isolated polypeptide or an isolated nucleic acid encoding a polypeptide having in some embodiments between about 70% and about 75%; in further embodiments between about 75% and about 80%; in further still embodiments between about 80% and 90%; or even more further between about 90% and about 99% of amino acids that are identical to (or homologous to) the amino acids of SEQ ID NO: 1 or SEQ ID NO: 3 or fragments thereof.

The percent identity or homology is determined with regard to the length of the relevant amino acid sequence. Therefore, if a polypeptide of the present invention is comprised within a larger polypeptide, the percent homology is determined with regard only to the portion of the polypeptide that corresponds to the polypeptide of the present invention and not the percent homology of the entirety of the larger polypeptide. “Percent identity” or “% identity,” with reference to nucleic acid sequences, refers to the percentage of identical nucleotides between at least two polynucleotide sequences aligned using the Basic Local Alignment Search Tool (BLAST) engine. See Tatusova et al. (1999) FEMS Microbiol Lett. 174:247-250. The BLAST engine is provided to the public by the National Center for Biotechnology Information (NCBI), Bethesda, Md. To align two polynucleotide sequences, the BLAST which employs the “blastn” program is used.

“Percent identity” or “% identity,” with reference to polypeptide sequences, refers to the percentage of identical amino acids between at least two polypeptide sequences aligned using the Basic Local Alignment Search Tool (BLAST) engine. See Tatusova et al. (1999) ibid. The BLAST engine is provided to the public by the National Center for Biotechnology Information (NCBI), Bethesda, Md. To align two polypeptide sequences, the BLAST which employs the “blastp” program is used.

In other embodiments, the present invention provides for an isolated nucleic acid encoding a polypeptide having between about 70% and about 75%; or more preferably between about 75% and about 80%; or more preferably between about 80% and 90%; or even more preferably between about 90% and about 99% of amino acids that are identical to the amino acids of SEQ ID NO: 1 or SEQ ID NO: 3 or fragments thereof.

In some embodiments, the nucleic acid segments, regardless of the length of the coding sequence itself, may be combined with other DNA sequences, such as promoters, enhancers, polyadenylation signals, additional restriction enzyme sites, multiple cloning sites, other coding segments, and the like. In some embodiments, for example, are recombinant nucleic acids comprising a nucleotide sequence that encodes amino acids of SEQ ID NO: 1, SEQ ID NO:3 or fragments thereof, operably linked to a heterologous promoter.

In certain embodiments the invention provides an isolated nucleic acid obtained by amplification from a template nucleic acid using a primer selected from the group consisting of SEQ ID NO: 21-24 or other appropriate primer that can be used with SEQ ID NO:2.

In some embodiments, a recombinant host cell comprising one of the nucleic acid sequences described. In some embodiments, a protein composition comprising one of the polypeptides described.

In some embodiments, are methods of treating a neuronal disorder, comprising: (a) delivering to a target neuron a nucleic acid expression vector that encodes a rhodopsin domain of a highly efficient, sodium specific, blue-shifted channelrhodopsin derived from algae, expressible in said target neuron, said vector comprising an open reading frame encoding the rhodopsin domain of a highly efficient, sodium specific, blue-shifted channelrhodopsin, operatively linked to a promoter sequence, and optionally, a transcriptional regulatory sequence; and (b) expressing said vector in said target neuron, wherein the expressed rhodopsin activates said target neuron upon exposure to light. In some embodiments, the rhodopsin domain is encoded by SEQ ID NO: 1.

In some embodiments, are methods of treating a neuronal disorder, comprising: (a) delivering to a target neuron a nucleic acid expression vector that encodes a rhodopsin domain of a channelrhodopsin derived from algae, expressible in said target neuron, said vector comprising an open reading frame encoding the rhodopsin domain of a channelrhodopsin, operatively linked to a promoter sequence, and optionally, a transcriptional regulatory sequence; and (b) expressing said vector in said target neuron, wherein the expressed rhodopsin activates said target neuron upon exposure to light. In some embodiments, the rhodopsin domain is encoded by SEQ ID NO: 1.

In some embodiments, are methods of restoring light sensitivity to a retina, comprising: (a) delivering to a retinal neuron a nucleic acid expression vector that encodes a rhodopsin domain of a highly efficient, sodium specific, blue-shifted channelrhodopsin derived from algae, expressible in the retinal neuron; said vector comprising an open reading frame encoding the rhodopsin domain of a highly efficient, sodium specific, blue-shifted channelrhodopsin operatively linked to a promoter sequence, and optionally, a transcriptional regulatory sequence; and (b) expressing said vector in said retinal neuron, wherein the expressed rhodopsin renders said retinal neuron photosensitive, thereby restoring light sensitivity to said retina or a portion thereof. In some embodiments, the rhodopsin domain is encoded by SEQ ID NO: 1.

In some embodiments, are methods of restoring light sensitivity to a retina, comprising: (a) delivering to a retinal neuron a nucleic acid expression vector that encodes a rhodopsin domain of a highly efficient, sodium specific, blue-shifted channelrhodopsin derived from algae, expressible in the retinal neuron; said vector comprising an open reading frame encoding the rhodopsin domain of a highly efficient, sodium specific, blue-shifted channelrhodopsin operatively linked to a promoter sequence, and optionally, a transcriptional regulatory sequence; and (b) expressing said vector in said retinal neuron, wherein the expressed rhodopsin renders said retinal neuron photosensitive, thereby restoring light sensitivity to said retina or a portion thereof. In some embodiments, the rhodopsin domain is encoded by SEQ ID NO: 1.

In some embodiments, are methods of restoring photosensitivity to a retina of a subject suffering from vision loss or blindness in whom retinal photoreceptor cells are degenerating or have degenerated and died, said method comprising: (a) delivering to the retina of said subject a nucleic acid vector that encodes a rhodopsin domain of a highly efficient, sodium specific, blue-shifted channelrhodopsin derived from algae expressible in a retinal neuron; said vector comprising an open reading frame encoding the rhodopsin domain of a highly efficient, sodium specific, blue-shifted channelrhodopsin operatively linked to a promoter sequence, and optionally, a transcriptional regulatory sequence; and (b) expressing said vector in said retinal neuron, wherein the expressed rhodopsin renders said retinal neuron photosensitive, thereby restoring photosensitivity to said retina or a portion thereof. In some embodiments, the rhodopsin domain is encoded by SEQ ID NO: 1.

In some embodiments, are methods of restoring photosensitivity to a retina of a subject suffering from vision loss or blindness in whom retinal photoreceptor cells are degenerating or have degenerated and died, said method comprising: (a) delivering to the retina of said subject a nucleic acid vector that encodes a rhodopsin domain of a highly efficient, sodium specific, blue-shifted channelrhodopsin derived from algae expressible in a retinal neuron; said vector comprising an open reading frame encoding the rhodopsin domain of a highly efficient, sodium specific, blue-shifted channelrhodopsin operatively linked to a promoter sequence, and optionally, a transcriptional regulatory sequence; and (b) expressing said vector in said retinal neuron, wherein the expressed rhodopsin renders said retinal neuron photosensitive, thereby restoring photosensitivity to said retina or a portion thereof. In some embodiments, the rhodopsin domain is encoded by SEQ ID NO: 1.

In some embodiments, are any of the disclosed methods, wherein the rhodopsin domain of a highly efficient, sodium specific, blue-shifted channelrhodopsin having the amino acid sequence of all or part of SEQ ID NOS: 1 or 3, or a biologically active fragment thereof that retains the biological activity of the encoded rhodopsin domain of a highly efficient, sodium specific, blue-shifted channelrhodopsin or a biologically active conservative amino acid substitution variant of SEQ ID NOS: 1 or 3 or of said fragment.

In some embodiments, are any of the disclosed methods, wherein the rhodopsin domain of a highly efficient, sodium specific, blue-shifted channelrhodopsin having the amino acid sequence of all or part of SEQ ID NOS: 1 or 3, or a biologically active fragment thereof that retains the biological activity of the encoded rhodopsin domain of a highly efficient, sodium specific, blue-shifted channelrhodopsin or a biologically active conservative amino acid substitution variant of SEQ ID NO: 1 or 3 or of said fragment.

In some embodiments, are any of the disclosed methods wherein the expression vectors include, but are not limited to, AAV viral vector. In some embodiments, are any of the disclosed methods wherein the promoter is a constitutive promoter. In some embodiments, are any of the disclosed methods wherein the constitutive promoter includes, but is not limited to, a CMV promoter or a hybrid CMV enhancer/chicken β-actin (CAG) promoter. In some embodiments, are any of the disclosed methods wherein the promoter includes, but is not limited to, an inducible and/or a cell type-specific promoter.

In some embodiments, a method of treating a neuronal disorder comprises:(a) delivering to a target neuron a nucleic acid expression vector that encodes a rhodopsin domain of a highly efficient, sodium specific, blue-shifted channelrhodopsin derived from algae, expressible in said target neuron; said vector comprising an open reading frame encoding the rhodopsin domain of a highly efficient, sodium specific, blue-shifted channelrhodopsin operatively linked to a promoter sequence, and optionally, transcriptional regulatory sequences; and (b) expressing the expression vector in the target neuron, wherein the expressed channelrhodopsin activates the target neuron upon exposure to light. In some embodiments an above-described expression vector also comprises one or more transcriptional regulatory sequences operably linked to the promoter and rhodopsin domain sequences. In some embodiments, the rhodopsin domain of a highly efficient, sodium specific, blue-shifted channelrhodopsin has the amino acid sequence of all or part of SEQ ID NOS: 1 or 3 and the rhodopsin domain sequences of SEQ ID NO:1, or a biologically active fragment thereof that retains the biological activity of the encoded rhodopsin domain of a channelrhodopsin or is a biologically active conservative amino acid substitution variant of SEQ ID NOS: 1 or 3 or of said fragment. In some embodiments, the expression vector comprises an AAV viral vector. In some embodiments, the promoter is a constitutive promoter. In some embodiments, the constitutive promoter is a CMV promoter or a hybrid CMV enhancer/chicken β-actin (CAG) promoter. In some embodiments, the promoter is an inducible and/or a cell type-specific promoter.

In some embodiments, a method of restoring light sensitivity to a retina comprises (a) delivering to a retinal neuron in a subject a nucleic acid expression vector that encodes a rhodopsin domain of a highly efficient, sodium specific, blue-shifted channelrhodopsin derived from algae, expressible in the retinal neuron; said expression vector comprising an open reading frame encoding the rhodopsin domain of a highly efficient, sodium specific, blue-shifted channelrhodopsin operatively linked to a promoter sequence, and optionally, one or more transcriptional regulatory sequences; and (b) expressing the expression vector in the retinal neuron, wherein the expressed rhodopsin renders the retinal neuron photosensitive, thereby restoring light sensitivity to the retina or a portion thereof. In some embodiments, the rhodopsin domain of a highly efficient, sodium specific, blue-shifted channelrhodopsin has the amino acid sequence of all or part of SEQ ID NOS: 1 or 3 and therhodopsin domain sequences of SEQ ID NO: 1, or a biologically active fragment thereof that retains the biological activity of the encoded rhodopsin domain of a highly efficient, sodium specific, blue-shifted channelrhodopsin or is a biologically active conservative amino acid substitution variant of SEQ ID NOS: 1 or 3 and the rhodopsin domain sequences of SEQ ID NO: 1, or of said fragment. In some embodiments, the expression vector comprises an AAV viral vector. In some embodiments, the promoter is a constitutive promoter. In some embodiments, the constitutive promoter is a CMV promoter or a hybrid CMV enhancer/chicken β-actin (CAG) promoter. In some embodiments, the promoter is an inducible and/or a cell type-specific promoter.

In some embodiments, a method of restoring photosensitivity to a retina of a subject suffering from vision loss or blindness in whom retinal photoreceptor cells are degenerating or have degenerated and died comprises: (a) delivering to the retina of the subject a nucleic acid expression vector that encodes a rhodopsin domain of a highly efficient, sodium specific, blue-shifted channelrhodopsin derived from algae expressible in retinal neurons; said expression vector comprising an open reading frame encoding the rhodopsin domain of a highly efficient, sodium specific, blue-shifted channelrhodopsin operatively linked to a promoter sequence, and optionally, transcriptional regulatory sequences; and (b) expressing the expression vector in the retinal neuron, wherein the expression of the rhodopsin renders the retinal neuron photosensitive, thereby restoring photosensitivity to said retina or a portion thereof. In some embodiments, the rhodopsin domain of a highly efficient, sodium specific, blue-shifted channelrhodopsin has the amino acid sequence of all or part of SEQ ID NOS: 1 or 3 and the rhodopsin domain sequences of SEQ ID NO: 1, or a biologically active fragment thereof that retains the biological activity of the encoded rhodopsin domain of a highly efficient, sodium specific, blue-shifted channelrhodopsin or is a biologically active conservative amino acid substitution variant of SEQ ID NOS: 1 or 3 and the rhodopsin domain sequences of SEQ ID NO: 1, or of said fragment. In some embodiments, the expression vector comprises an AAV viral vector. In some embodiments, the promoter is a constitutive promoter. In some embodiments, the constitutive promoter is a CMV promoter or a hybrid CMV enhancer/chicken β-actin (CAG) promoter. In other embodiments, the promoter is an inducible and/or a cell type-specific promoter.

To identify algal species were screened for candidates of new channelrhodopsins with desirable characteristics, using the photoelectrophysiological population assay for recording rhodopsin-mediated photocurrents. EST and homology cloning was also used to identify new channelopsin sequences in several algal species.

Exemplified below are the specifics of the process. PsChR, (SEQ ID NO: 1) derived from the channelrhodopsin of Platymonas (Tetraselmis) subcordiformis (cDNA nucleic acid in SEQ ID NO: 2 and translated amino acid in SEQ ID NO: 3. EMBL entry JX983143) as well as DsChR1 (SEQ ID NO: 4) derived from the channelrhodopsin 1 of Dunaliella salina (EMBL entry JQ241364); CaChR1 (SEQ ID NO: 5) derived from the channelrhodopsin 1 of Chlamydomonas augustae (SEQ ID NO: 13, EMBL entry JN596951); MChR1, (SEQ ID NO: 6) derived from the channelrhodopsin 1 of Mesostigma viride (SEQ ID NO: 14, EMBL entry JF922293); and CrChR2 (SEQ ID NO: 7) derived from the channelrhodopsin 2 of Chlamydomonas reinhardtii (SEQ ID NO: 11, EMBL entry AF508966) were cloned and expressed.

PsChR, (SEQ ID NO: 1) was thus established as having the most blue-shifted spectral sensitivities so far reported, matches or surpasses known channelrhodopsins' channel kinetics, undergoes minimal inactivation upon sustained illumination, and exhibits pH-independent spectral sensitivity of membrane depolarization. This combination of properties makes PsChR, (SEQ ID NO: 1) particularly useful as a more precise and versatile agent for control of neuronal activity as well as for optogenetic uses.

In some embodiments, the cloning and analysis of new channelopsins from a phylogenetically different alga expands the set of the currently available optogenetic techniques by introducing a fast, blue-shifted channelrhodopsin species, and also contributes to our understanding of the sequence determinants of channelrhodopsin function. Furthermore, retinal neurons not normally sensitive to direct light located in the retinas of blind mice, such as retinal ganglion cells (RGCs) and bipolar cells, can respond to light when a green algae protein called channelrhodopsin-2 (ChR2), or a biologically active fragment or a conservative amino acid substitution variant thereof, is inserted into the neuronal cell membranes. In some embodiments the described channelrhodopsins may be used to transform retinal neurons not normally sensitive to direct light located in the retinas. In some embodiments, are methods and compositions of a novel channelrhodopsin 2 domain, identified as PsChR, (SEQ ID NO: 1) derived from the channelrhodopsin of Platymonas (Tetraselmis) subcordiformis (SEQ ID NO: 3. EMBL entry JX983143).

In some embodiments, molecular engineered variants (some with improved activity) of the described a highly efficient, sodium specific, blue-shifted channelrhodopsins by site-specific mutagenesis and chimera construction. In some embodiments, the channelrhodopsins serve as receptors for phototaxis and the photophobic response. Their photoexcitation initiates depolarization of the cell membrane.

In some embodiments, the rhodopsin domains of several channelrhodopsins were cloned and determined to have channel activity when they were expressed in mammalian HEK293 cells. Using these methods new channelrhodopsin variants, were determined to have improved properties with regards to, among other things, optogenetics. Currents generated by PsChR in HEK293 cells show higher amplitude, smaller inactivation, and faster peak recovery than those generated by CrChR2, the molecule of choice in most optogenetic studies, whereas their kinetics is similarly fast. The higher current amplitude of PsChR is due to its higher unitary conductance, as estimated by stationary noise analysis.

It is shown in the examples below that PsChR is expressed in cultured hippocampal neurons and can be used to drive their spiking activity by light excitation. Moreover, a faster peak recovery of PsChR-generated currents provides an advantage in certain optogenetic applications.

PsChR has a more blue-shifted spectral sensitivity than the previously available rhodopsin domains with faster current kinetics and smaller inactivation, and faster peak recovery all of which makes these rhodopsin domains better suited for, among other things, rapid control of neuronal activity.

PsChR (SEQ ID NO: 1) which was derived from Platymonas (Tetraselmis) subcordiformis. It is shown in the examples below that PsChR expressed in cultured hippocampal neurons can be used to drive their spiking activity by light excitation. Moreover, a faster peak recovery of PsChR-generated currents provides an advantage in certain optogenetic applications.

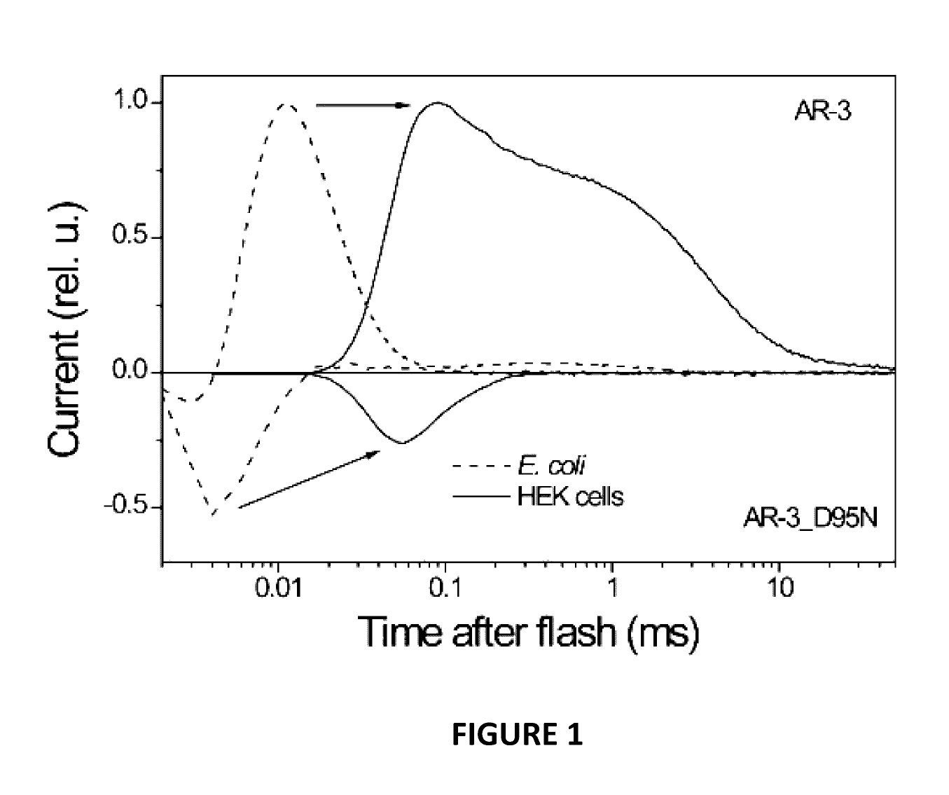

In previously identified algal channelrhodopsins (ChR), the longer-wavelength (green) absorbing ChR of the pair was designated as ChR1, and the shorter-wavelength (blue) absorbing as ChR2. Alignment of the fourteen currently reported ChR sequences (Suppl. FIG. 1) do not divide them in two distinct groups. Therefore, when only one ChR species of a presumed pair is known in a particular organism, it is difficult to assign it to ChR group 1 or 2 from the sequence information alone. However, matching the spectral properties of PsChR with the action spectrum of photoreceptor currents in P. subcordiformis clearly indicates that it is the blue-shifted member of the pair, and consequently can be classified as PsChR2, despite it being the only so far identified ChR in this alga. PsChR absorption is the most blue-shifted of all known ChRs. Generally, for optogenetic tools long-wavelength absorption is preferable to minimize scattering of light by biological tissues and its absorption by hemoglobin. However, the blue-shifted spectrum of PsChR is ideal for combinatorial applications with long wavelength-absorbing rhodopsin pumps or fluorescent indicators.

All known ChRs are predominantly proton channels, so their use for optogenetics may lead to undesirable acidification of the cytoplasm. The higher selectivity of PsChR for Na+ ions over protons may circumvent this side effect. Both PsChR and MvChR1 show high Na+ selectivity, but its mechanisms appear to be fundamentally different in these proteins. In MvChR1, which uniquely contains an alanine residue in place of His134 (CrChR2 numbering), Na+ conductance is inhibited by protons, as it is in the H134R/S mutants of CrChR2. On the other hand, no such inhibition was found in PsChR, in which His134 is conserved (Suppl. FIG. 1).

PsChR was expressed in cultured hippocampal neurons and can be used to drive their spiking activity by light excitation. Moreover, a faster peak recovery of PsChR-generated currents provides an advantage in certain optogenetic applications. Recently it has been demonstrated that the level of expression and, correspondingly, the amplitude of channel currents generated by CrChR2 in animal cells dramatically increase upon the addition of exogenous retinal (67) however this increase was even larger for PsChR than for CrChR2. Retinal content of some heterologous systems, such as C. elegans or Drosophila, is so low that even CrChR2 cannot be used there without the addition of exogenous retinal. On the other hand, the intact mammalian brain may have enough endogenous retinal to reconstitute fully functional PsChR.

The characterization of PsChR augments current understanding of functional mechanisms of ChRs and reveal its potentially useful properties as an optogenetic tool. Taking into account the great number and diversity of phototactic algal species in which the existence of ChRs is predicted, comparative analysis of different native ChR variants may be as beneficial as bioengineering of select model molecules both for basic research into their structure-function relationships and for optimization of their biotechnology applications.

One of the major challenges for optogenetic applications, especially in living animals, are scattering of the stimulating light by biological tissues and its absorption by hemoglobin. Optogenetic tools with long-wavelength absorption would exhibit minimal light attenuation from these effects, but most microbial rhodopsins do not fall into this category. For instance, the absorption maximum of ChR2, which possesses several other useful properties and is thereby most frequently used as a depolarizing tool in optogenetics, is 470 nm. Several approaches have been taken to attempt to acquire blue-shifted variants to reduce the light-attenuation by scatter and absorption in tissue: (i) searching for natural blue-shifted channelrhodopsin variants in different algae (such as those described herein); (ii) chimera construction; and (iii) site-directed mutagenesis. All of these approaches have in common modification of the apoprotein, and all have proved somewhat successful, although in some cases a desired spectral shift was accompanied by negative effects such as slowing down of the current kinetics, or a decrease in the current amplitude.

Long-wavelength absorption by optogenetic tools is generally considered desirable to increase the penetration depth of the stimulus light by minimizing tissue scattering and absorption by hemoglobin. In some embodiments, the long-wavelength sensitivity of optogenetic microbial rhodopsins is enhanced using 3,4-Dehydroretinal (A2 retinal). A2 retinal (3,4-dehydroretinal) is a natural retinoid, its 11-cis form being found in photoreceptor cells of certain invertebrates, fish and amphibians, where it may constitute the only retinal, or an additional chromophore to A1 retinal. The presence of an additional double bond in the β-ionone ring of the chromophore results in pigments that absorb light at longer wavelengths, as compared to those formed with A1 (regular) retinal. Variations in A1/A2 ratio cause natural adaptive tuning of spectral sensitivity of vision in the organisms during adaptation to external conditions. Reconstitution of bleached microbial rhodopsins (bacteriorhodopsin, halorhodopsin, sensory rhodopsins I and II) in vitro with all-trans A2 retinal also shifts their absorption spectra to longer wavelengths. In some embodiments, spectral properties of optogenetic tools were modified by incorporation of all-trans A2 retinal. The addition of A2 retinal, both ion pumps and channelrhodopsins form functional pigments with significantly blue-shifted absorption.

In some embodiments, the long-wavelength sensitivity of optogenetic microbial rhodopsins is enhanced using 3,4-Dehydroretinal (A2 retinal). In some embodiments, chromophore substitution provides a complementary strategy to improve the efficiency of optogenetic tools. Substitution of A1 by A2 retinal significantly shifts the spectral sensitivity of tested rhodopsins to longer wavelengths without altering other aspects of their function.

Channelrhodopsin Amino Acid Sequences: The peptide amino acid sequences that can be used in various embodiments including the channelrhodopsin amino acid sequences described herein (SEQ ID NOS: 1 or 3), as well as analogues and derivatives thereof and functional fragments such as but not limited to the rhodopsin domain. In fact, in some embodiments the any desired peptide amino acid sequences encoded by particular nucleotide sequences can be used, as is the use of any polynucleotide sequences encoding all, or any portion, of desired peptide amino acid sequences. The degenerate nature of the genetic code is well-known, and, accordingly, each channelrhodopsin peptide amino acid-encoding nucleotide sequence is generically representative of the well-known nucleic acid “triplet” codon, or in many cases codons, that can encode the amino acid. As such, as contemplated herein, the channelrhodopsin peptide amino acid sequences described herein, when taken together with the genetic code (see, e.g., “Molecular Cell Biology”, Table 4-1 at page 109 (Darnell et al., eds., W. H. Freeman & Company, New York, NY, 1986)), are generically representative of all the various permutations and combinations of nucleic acid sequences that can encode such amino acid sequences.

Such functionally equivalent peptide amino acid sequences (conservative substitutions) include, but are not limited to, additions or substitutions of amino acid residues within the amino acid sequences encoded by a nucleotide sequence, but that result in a silent change, thus producing a functionally equivalent gene product. Amino acid substitutions may be made on the basis of similarity in polarity, charge, solubility, hydrophobicity, hydrophilicity, and/or the amphipathic nature of the residues involved. For example: nonpolar (hydrophobic) amino acids include alanine, leucine, isoleucine, valine, proline, phenylalanine, tryptophan, and methionine; polar neutral amino acids include glycine, serine, threonine, cysteine, tyrosine, asparagine, and glutamine; positively charged (basic) amino acids include arginine, lysine, and histidine; and negatively charged (acidic) amino acids include aspartic acid and glutamic acid.

Conservative amino acid substitutions may alternatively be made on the basis of the hydropathic index of amino acids. Each amino acid has been assigned a hydropathic index on the basis of its hydrophobicity and charge characteristics. They are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5). The use of the hydropathic amino acid index in conferring interactive biological function on a protein is understood in the art (Kyte and Doolittle, J. Mol. Biol. 157:105-132, 1982). It is known that in certain instances, certain amino acids may be substituted for other amino acids having a similar hydropathic index or score and still retain a similar biological activity. In making changes based upon the hydropathic index, in certain embodiments the substitution of amino acids whose hydropathic indices are within ±2 is included, while in other embodiments amino acid substitutions that are within ±1 are included, and in yet other embodiments amino acid substitutions within ±0.5 are included.

Conservative amino acid substitutions may alternatively be made on the basis of hydrophilicity, particularly where the biologically functional protein or peptide thereby created is intended for use in immunological embodiments. In certain embodiments, the greatest local average hydrophilicity of a protein, as governed by the hydrophilicity of its adjacent amino acids, correlates with its immunogenicity and antigenicity, i.e., with a biological property of the protein. The following hydrophilicity values have been assigned to these amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5) and tryptophan (−3.4). In making changes based upon similar hydrophilicity values, in certain embodiments the substitution of amino acids whose hydrophilicity values are within ±2 is included, in certain embodiments those that are within ±1 are included, and in certain embodiments those within ±0.5 are included.

Fusion Proteins: The use of fusion proteins in which a polypeptide or peptide, or a truncated or mutant version of peptide is fused to an unrelated protein, polypeptide, or peptide, and can be designed on the basis of the desired peptide encoding nucleic acid and/or amino acid sequences described herein. Such fusion proteins include, but are not limited to: IgFc fusions, which stabilize proteins or peptides and prolong half-life in vivo; fusions to any amino acid sequence that allows the fusion protein to be anchored to the cell membrane; or fusions to an enzyme, fluorescent protein, or luminescent protein that provides a marker function.

In certain embodiments, a fusion protein may be readily purified by utilizing an antibody that selectively binds to the fusion protein being expressed. In alternate embodiments, a fusion protein may be purified by subcloning peptide encoding nucleic acid sequence into a recombination plasmid, or a portion thereof, is translationally fused to an amino-terminal (N-terminal) or carboxy-terminal (C-terminal) tag consisting of six histidine residues (a “His-tag”; see, e.g., Janknecht et al., Proc. Natl. Acad. Sci. USA 88:8972-8976, 1991). Extracts from cells expressing such a construct are loaded onto Ni²⁺ nitriloacetic acid-agarose columns, and histidine-tagged proteins are selectively eluted with imidazole-containing buffers.

Recombinant Expression: While the desired peptide amino acid sequences described can be chemically synthesized (see, e.g., “Proteins: Structures and Molecular Principles” (Creighton, ed., W. H. Freeman & Company, New York, N.Y., 1984)), large polypeptides sequences may advantageously be produced by recombinant DNA technology using techniques well-known in the art for expressing nucleic acids containing a nucleic acid sequence that encodes the desired peptide. Such methods can be used to construct expression vectors containing peptide encoding nucleotide sequences and appropriate transcriptional and translational control signals. These methods include, for example, in vitro recombinant DNA techniques, synthetic techniques, and in vivo genetic recombination (see, e.g., “Molecular Cloning, A Laboratory Manual”, supra, and “Current Protocols in Molecular Biology”, supra). Alternatively, RNA and/or DNA encoding desired peptide encoding nucleotide sequences may be chemically synthesized using, for example, synthesizers (see, e.g., “Oligonucleotide Synthesis: A Practical Approach” (Gait, ed., IRL Press, Oxford, United Kingdom, 1984)).

A variety of host-expression vector systems may be utilized to express peptide encoding nucleotide sequences. When the desired peptide or polypeptide is soluble or a soluble derivative, the peptide or polypeptide can be recovered from the host cell culture, i.e., from the host cell in cases where the peptide or polypeptide is not secreted, and from the culture media in cases where the peptide or polypeptide is secreted by the host cell. However, suitable expression systems also encompass engineered host cells that express the desired polypeptide or functional equivalents anchored in the cell membrane. Purification or enrichment of the desired peptide from such expression systems can be accomplished using appropriate detergents and lipid micelles, and methods well-known to those skilled in the art. Furthermore, such engineered host cells themselves may be used in situations where it is desired not only to retain the structural and functional characteristics of the peptide, but to assess biological activity, e.g., in certain drug screening assays.

In certain applications, transient expression systems are desired. However, for long-term, high-yield production of recombinant proteins or peptides, stable expression is generally preferred. For example, cell lines that stably express the desired protein, polypeptide, peptide, or fusion protein may be engineered. Rather than using expression vectors that contain viral origins of replication, host cells can be transformed with DNA controlled by appropriate expression control elements (e.g., promoter, enhancer sequences, transcription terminators, polyadenylation sites, etc.), and a selectable marker. Following the introduction of the foreign DNA, engineered cells are allowed to grow for about 1-2 days in an enriched media, and then switched to a selective media. The selectable marker in the recombinant plasmid confers resistance to the selection, and allows cells to stably integrate the plasmid into their chromosomes and grow to form foci, which in turn can be cloned and expanded into cell lines. This method may advantageously be used to engineer cell lines that express the desired gene products or portions thereof. Such engineered cell lines may be particularly useful in screening and evaluation of compounds that affect the endogenous activity of a desired protein, polypeptide or peptide.

A number of selection systems may be used, including, but not limited to, the herpes simplex virus thymidine kinase (Wigler et al., Cell 11:223-232, 1977), hypoxanthine-guanine phosphoribosyltransferase (Szybalska and Szybalski, Proc. Natl. Acad. Sci. USA 48:2026-2034, 1962), and adenine phosphoribosyltransferase (Lowy et al., Cell 22:817-823, 1980) genes, which can be employed in tk⁻, hgprt⁻ or aprt⁻ cells, respectively. Anti-metabolite resistance can also be used as the basis of selection for the following genes: dihydrofolate reductase (dhfr), which confers resistance to methotrexate (Wigler et al., Proc. Natl. Acad. Sci. USA 77:3567-3570, 1980, and O'Hare et al., Proc. Natl. Acad. Sci. USA 78:1527-1531, 1981); guanine phosphoribosyl transferase (gpt), which confers resistance to mycophenolic acid (Mulligan and Berg, Proc. Natl. Acad. Sci. USA 78:2072-2076, 1981); neomycin phosphotransferase (neo), which confers resistance to the aminoglycoside G-418 (Colbere-Garapin et al., J. Mol. Biol. 150:1-14, 1981); and hygromycin B phosphotransferase (hpt), which confers resistance to hygromycin (Santerre et al., Gene 30:147-156, 1984).

Host cells/expression systems that may be used for purpose of providing compositions to be used in the disclosed methods include, but are not limited to, microorganisms such as bacteria (e.g., E. coli, B. subtilis) transformed with a recombinant bacteriophage DNA, plasmid DNA, or cosmid DNA expression vector containing a desired peptide encoding nucleotide sequence; yeast (e.g., Saccharomyces cerevisiae, Pichia pastoris) transformed with a recombinant yeast expression vector containing a desired peptide encoding nucleotide sequence; insect cell systems infected with a recombinant virus expression vector (e.g., baculovirus) containing a desired peptide encoding nucleotide sequence; plant cell systems infected with a recombinant virus expression vector (e.g., cauliflower mosaic virus, CaMV; tobacco mosaic virus, TMV), or transformed with a recombinant plasmid expression vector (e.g., Ti plasmid), containing a desired peptide encoding nucleotide sequence; or mammalian cell systems (e.g., COS, CHO, BHK, 293, 3T3) harboring a recombinant expression construct containing a desired peptide encoding nucleotide sequence and a promoter derived from the genome of mammalian cells (e.g., metallothionein promoter) or from mammalian viruses (e.g., the adenovirus late promoter, the vaccinia virus 7.5K promoter).

In bacterial systems, a number of different expression vectors may be advantageously selected depending upon the use intended for the desired gene product being expressed. For example, when a large quantity of such a protein is to be produced, such as for the generation of pharmaceutical compositions comprising a desired peptide, or for raising antibodies to the protein, vectors that direct the expression of high levels of fusion protein products that are readily purified may be desirable. Such vectors include, but are not limited to: the E. coli expression vector pUR278 (Ruther and Müller-Hill, EMBO J. 2:1791-1794, 1983), in which a desired peptide encoding sequence may be ligated individually into the vector in frame with the lacZ coding region so that a fusion protein is produced; pIN vectors (Inouye and Inouye, Nucleic Acids Res. 13:3101-3110, 1985, and Van Heeke and Schuster, J. Biol. Chem. 264:5503-5509, 1989); and the like. pGEX vectors (GE Healthcare, Piscataway, N.J.) may also be used to express a desired peptide moiety as a fusion protein with glutathione S-transferase (GST). In general, such fusion proteins are soluble and can easily be purified from lysed cells by adsorption to glutathione-agarose beads, followed by elution in the presence of free glutathione. The pGEX vectors are designed to include thrombin or factor Xa protease cleavage sites so that the cloned desired peptide encoding gene product can be released from the GST moiety.

In an exemplary insect system, Autographa californica nuclear polyhedrosis virus (AcNPV) is used as a vector to express a desired peptide encoding sequence. The virus grows in Spodoptera frugiperda cells. A desired peptide encoding sequence may be cloned individually into a non-essential region (for example the polyhedrin gene) of the virus, and placed under control of an AcNPV promoter (for example the polyhedrin promoter). Successful insertion of a desired peptide encoding sequence will result in inactivation of the polyhedrin gene and production of non-occluded recombinant virus (i.e., virus lacking the proteinaceous coat coded for by the polyhedrin gene). The recombinant viruses are then used to infect Spodoptera frugiperda cells in which the inserted polynucleotide is expressed (see, e.g., Smith et al., J. Virol. 46:584-593, 1983, and U.S. Pat. No. 4,215,051).

In mammalian host cells, a number of viral-based expression systems may be utilized. In cases where an adenovirus is used as an expression vector, a desired peptide encoding nucleotide sequence may be ligated to an adenovirus transcription/translation control complex, e.g., the late promoter and tripartite leader sequence. This chimeric sequence may then be inserted in the adenovirus genome by in vitro or in vivo recombination. Insertion in a non-essential region of the viral genome (e.g., region E1 or E3) will result in a recombinant virus that is viable and capable of expressing desired peptide products in infected hosts (see, e.g., Logan and Shenk, Proc. Natl. Acad. Sci. USA 81:3655-3659, 1984). Specific initiation signals may also be required for efficient translation of inserted desired peptide encoding nucleotide sequences. These signals include the ATG initiation codon and adjacent sequences. In some cases exogenous translational control signals, including, perhaps, the ATG initiation codon, may be provided. Furthermore, the initiation codon should be in phase with the reading frame of the desired peptide encoding coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see, e.g., Nevins, CRC Crit. Rev. Biochem. 19:307-322, 1986).

In yeast, a number of vectors containing constitutive or inducible promoters may be used. For a review, see, e.g., “Current Protocols in Molecular Biology”, supra, Ch. 13, Bitter et al., Meth. Enzymol. 153:516-544, 1987, “DNA Cloning”, Vol. II, Ch. 3 (Glover, ed., IRL Press, Washington, D.C., 1986); Bitter, Meth. Enzymol. 152:673-684, 1987, The Molecular Biology of the Yeast Saccharomyces: Life Cycle and Inheritance” (Strathern et al., eds., Cold Spring Harbor Press, Cold Spring Harbor, N.Y., 1981), and The Molecular Biology of the Yeast Saccharomyces: Metabolism and Gene Expression” (Strathern et al., eds., Cold Spring Harbor Press, Cold Spring Harbor, N.Y., 1982).

In plants, a variety of different plant expression vectors can be used, and expression of a desired peptide encoding sequence may be driven by any of a number of promoters. For example, viral promoters such as the 35S RNA or 19S RNA promoters of CaMV (Brisson et al., Nature 310:511-514, 1984), or the coat protein promoter of TMV (Takamatsu et al., EMBO J. 6:307-311, 1987) may be used. Alternatively, plant promoters such as the promoter of the small subunit of RUBISCO (Coruzzi et al., EMBO J. 3:1671-1679, 1984, and Broglie et al., Science 224:838-843, 1984), or heat shock promoters, e.g., soybean hsp17.5-E or hsp17.3-B (Gurley et al., Mol. Cell. Biol. 6:559-565, 1986) may be used. These constructs can be introduced into plant cells using, for example, Ti plasmids, Ri plasmids, plant virus vectors, direct DNA transformation, microinjection, or electroporation. For reviews of such techniques, see, e.g., Weissbach and Weissbach, in “Methods in Plant Molecular Biology”, Section VIII (Schuler and Zielinski, eds., Academic Press, Inc., New York, N.Y., 1988), and “Plant Molecular Biology”, 2^{nd} Ed., Ch. 7-9 (Grierson and Covey, eds., Blackie & Son, Ltd., Glasgow, Scotland, United Kingdom, 1988).

In addition, a host cell strain may be chosen that modulates the expression of the inserted desired peptide encoding sequence, or modifies and processes the desired peptide encoding nucleic acid sequence in a desired fashion. Such modifications (e.g., glycosylation) and processing (e.g., cleavage) of protein products may affect certain functions of the protein. Different host cells have characteristic and specific mechanisms for post-translational processing and modification of proteins and peptides. Appropriate cell lines or host systems can be chosen to ensure the correct or desired modification and processing of the desired protein, polypeptide, or peptide expressed. To this end, eukaryotic host cells that possess the cellular machinery for desired processing of the primary transcript, and glycosylation and/or phosphorylation of desired peptide encoding nucleic acid sequence be used. Such mammalian host cells include, but are not limited to, Chinese hamster ovary (CHO), VERO, baby hamster kidney (BHK), HeLa, monkey kidney (COS), MDCK, 293, 3T3, WI38, human hepatocellular carcinoma (e.g., Hep G2),and U937 cells.

Compositions as Therapeutics: The use of channelrhodopsins, or active fragments thereof such as but not limited to the rhodopsin domain as therapeutics. In certain embodiments the presently disclosed compositions and are used to improve optogenetic techniques and applications as well as can be used to aid in diagnosis, prevention, and/or treatment of among other things neuron mediated disorders, neurologic disorders (such as Parkinson's disease) and as therapy for ocular disorders.

In certain embodiments the presently disclosed compositions can be administered in combination with one or more additional compounds or agents (“additional active agents”) for the treatment, management, and/or prevention of among other things neuron mediated disorders, neurologic disorders (such as Parkinson's disease) and as therapy for ocular disorders. Such therapies can be administered to a patient at therapeutically effective doses to treat or ameliorate, among other things, neuron mediated disorders, neurologic disorders (such as Parkinson's disease) and as therapy for ocular disorders. A therapeutically effective dose refers to that amount of the compound sufficient to result in any delay in onset, amelioration, or retardation of disease symptoms.

Toxicity and therapeutic efficacy of such compositions can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index, expressed as the ratio LD₅₀/ED₅₀. Compositions that exhibit large therapeutic indices are preferred. Compounds that exhibit toxic side effects may be used in certain embodiments, however, care should usually be taken to design delivery systems that target such compositions preferentially to the site of affected tissue, in order to minimize potential damage to uninfected cells and, thereby, reduce side effects.

Data obtained from cell culture assays and animal studies can be used in formulating a range of dosages for use in humans. The dosages of such compositions lie preferably within a range of circulating concentrations that include the ED₅₀ with little or no toxicity. The dosage may vary within this range depending on the dosage form employed and the route of administration utilized. For any composition, the therapeutically effective dose can be estimated initially from cell culture assays. A dose may be formulated in animal models to achieve a circulating plasma concentration range that includes the IC₅₀ (i.e., the concentration of the test composition that achieves a half-maximal inhibition of symptoms) as determined in cell culture. Such information can be used to more accurately determine useful doses in humans. Plasma levels may be measured, for example, by high performance liquid chromatography.

When the therapeutic treatment of among other things neurologic disorders (such as Parkinson's disease) and as therapy for ocular disorders is contemplated, the appropriate dosage may also be determined using animal studies to determine the maximal tolerable dose, or MTD, of a bioactive agent per kilogram weight of the test subject. In general, at least one animal species tested is mammalian. Those skilled in the art regularly extrapolate doses for efficacy and avoiding toxicity to other species, including human. Before human studies of efficacy are undertaken, Phase I clinical studies help establish safe doses.

Additionally, the bioactive agent may be coupled or complexed with a variety of well established compositions or structures that, for instance, enhance the stability of the bioactive agent, or otherwise enhance its pharmacological properties (e.g., increase in vivo half-life, reduce toxicity, etc.).

Such therapeutic agents can be administered by any number of methods known to those of ordinary skill in the art including, but not limited to, inhalation, subcutaneous (sub-q), intravenous (I.V.), intraperitoneal (I.P.), intramuscular (I.M.), or intrathecal injection, or topically applied (transderm, ointments, creams, salves, eye drops, and the like), as described in greater detail below.

Pharmaceutical Compositions: Pharmaceutical compositions for use in accordance with the presently described compositions may be formulated in conventional manners using one or more physiologically acceptable carriers or excipients.

The pharmaceutical compositions can comprise formulation materials for modifying, maintaining, or preserving, for example, the pH, osmolarity, viscosity, clarity, color, isotonicity, odor, sterility, stability, rate of dissolution or release, adsorption or penetration of the composition. Suitable formulation materials include, but are not limited to: amino acids (for example, glycine, glutamine, asparagine, arginine and lysine); antimicrobials; antioxidants (for example, ascorbic acid, sodium sulfite and sodium hydrogen-sulfite); buffers (for example, borate, bicarbonate, Tris-HCl, citrates, phosphates and other organic acids); bulking agents (for example, mannitol and glycine); chelating agents (for example, ethylenediamine tetraacetic acid (EDTA)); complexing agents (for example, caffeine, polyvinylpyrrolidone, beta-cyclodextrin, and hydroxypropyl-beta-cyclodextrin); fillers; monosaccharides, disaccharides, and other carbohydrates (for example, glucose, mannose and dextrins); proteins (for example, serum albumin, gelatin and immunoglobulins); coloring, flavoring, and diluting agents; emulsifying agents; hydrophilic polymers (for example, polyvinylpyrrolidone); low molecular weight polypeptides; salt-forming counterions (for example, sodium); preservatives (for example, benzalkonium chloride, benzoic acid, salicylic acid, thimerosal, phenethyl alcohol, methylparaben, propylparaben, chlorhexidine, sorbic acid and hydrogen peroxide); solvents (for example, glycerin, propylene glycol and polyethylene glycol); sugar alcohols (for example, mannitol and sorbitol); suspending agents; surfactants or wetting agents (for example, pluronics, PEG, sorbitan esters, polysorbates (for example, polysorbate 20 and polysorbate 80), triton, tromethamine, lecithin, cholesterol, and tyloxapal); stability enhancing agents (for example, sucrose and sorbitol); tonicity enhancing agents (for example, alkali metal halides (for example, sodium or potassium chloride), mannitol, and sorbitol); delivery vehicles; diluents; excipients; and pharmaceutical adjuvants (“Remington's Pharmaceutical Sciences”, 18^{th} Ed. (Gennaro, ed., Mack Publishing Company, Easton, Pa., 1990)).

Additionally, the described therapeutic peptides can be linked to a half-life extending vehicle. Certain exemplary half-life extending vehicles are known in the art, and include, but are not limited to, the Fc domain, polyethylene glycol, and dextran (see, e.g., PCT Patent Application Publication No. WO 99/25044).

These agents may be formulated for parenteral administration by injection, e.g., by bolus injection or continuous infusion. Formulations for injection may be presented in unit dosage form, e.g., in ampules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The agents may also be formulated as compositions for rectal administration such as suppositories or retention enemas, e.g., containing conventional suppository bases such as cocoa butter or other glycerides.

In addition to the formulations described previously, the agents may also be formulated as a depot preparation. Such long acting formulations may be administered by implantation (for example subcutaneously or intramuscularly) or by intramuscular injection. For example, agents may be formulated with suitable polymeric or hydrophobic materials (for example as an emulsion in an acceptable oil), ion exchange resins, or as sparingly soluble derivatives, for example, as a sparingly soluble salt. The compositions may, if desired, be presented in a pack or dispenser device, which may contain one or more unit dosage forms containing the active ingredient. The pack may for example comprise metal or plastic foil, such as a blister pack. The pack or dispenser device may be accompanied by instructions for administration.

Active compositions can be administered by controlled release means or by delivery devices that are well-known to those of ordinary skill in the art. Examples include, but are not limited to, those described in U.S. Pat. Nos. 3,845,770, 3,916,899, 3,536,809, 3,598,123, 4,008,719, 5,674,533, 5,059,595, 5,591,767, 5,120,548, 5,073,543, 5,639,476, 5,354,556, and 5,733,566. Such dosage forms can be used to provide slow or controlled-release of one or more active ingredients using, for example, hydropropylmethyl cellulose, other polymer matrices, gels, permeable membranes, osmotic systems, multilayer coatings, microparticles, liposomes, microspheres, or a combination thereof, to provide the desired release profile in varying proportions. Exemplary sustained release matrices include, but are not limited to, polyesters, hydrogels, polylactides (see, e.g., U.S. Pat. No. 3,773,919 and European Patent Application Publication No. EP 058,481), copolymers of L-glutamic acid and gamma ethyl-L-glutamate (see, e.g., Sidman et al., Biopolymers 22:547-556, 1983), poly (2-hydroxyethyl-methacrylate) (see, e.g., Langer et al., J. Biomed. Mater. Res. 15:167-277, 1981, and Langer, Chemtech 12:98-105, 1982), ethylene vinyl acetate (Langer et al., supra), and poly-D(−)-3-hydroxybutyric acid (European Patent Application Publication No. EP 133,988). Sustained release compositions may include liposomes, which can be prepared by any of several methods known in the art (see, e.g., Eppstein et al., Proc. Natl. Acad. Sci. USA 82:3688-3692, 1985, and European Patent Application Publication Nos. EP 036,676, EP 088,046, and EP 143,949). Suitable controlled-release formulations known to those of ordinary skill in the art, including those described herein, can be readily selected for use with the presently disclosed compositions. Certain embodiments encompass single unit dosage forms suitable for oral administration such as, but not limited to, tablets, capsules, gelcaps, and caplets that are adapted for controlled-release.

All controlled-release pharmaceutical products have a common goal of improving therapy over that achieved by their non-controlled counterparts. Ideally, use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood levels of the drug, and can thus affect the occurrence of side (e.g., adverse) effects.

Most controlled-release formulations are designed to initially release an amount of active ingredient that promptly produces the desired therapeutic effect, and gradually and continually release other amounts of active ingredient to maintain this level of therapeutic or prophylactic effect over an extended period of time. In order to maintain this relatively constant level of active ingredient in the body, the drug must be released from the dosage form at a rate that will replace the amount of active ingredient being metabolized and excreted from the body. Controlled-release of an active ingredient can be stimulated by various conditions including, but not limited to, pH, temperature, enzymes, water, or other physiological conditions or compositions.

In some cases, active ingredients of the disclosed methods and compositions are preferably not administered to a patient at the same time or by the same route of administration. Therefore, in some embodiments are kits that, when used by the medical practitioner, can simplify the administration of appropriate amounts of active ingredients to a patient.

A typical kit comprises a single unit dosage form of one or more of the therapeutic agents disclosed, alone or in combination with a single unit dosage form of another agent that may be used in combination with the disclosed compositions. Disclosed kits can further comprise devices that are used to administer the active ingredients. Examples of such devices include, but are not limited to, syringes, drip bags, patches, and inhalers.

Disclosed kits can further comprise pharmaceutically acceptable vehicles that can be used to administer one or more active ingredients. For example, if an active ingredient is provided in a solid form that must be reconstituted for parenteral administration, the kit can comprise a sealed container of a suitable vehicle in which the active ingredient can be dissolved to form a particulate-free sterile solution that is suitable for parenteral administration. Examples of pharmaceutically acceptable vehicles include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate. However, in specific embodiments, the disclosed formulations do not contain any alcohols or other co-solvents, oils or proteins.

Channelrhodopsin Nucleic Acid Sequences: Channelrhodopsin nucleic acid sequences for use in the disclosed methods and compositions include, but are not limited to, the active portion of the presently disclosed algal derived blue-shifted channelrhodopsins PsChR (amino acid SEQ ID NO: 3, and cDNA sequence SEQ ID NO: 2) , including but not limited to those described, such as but not limited to the nucleic acid sequences that encode the rhodopsin domain, an active portion of the presently disclosed algal derived blue-shifted channelrhodopsins, such as but not limited to the rhodopsin domains disclosed (SEQ ID NO: 1).

In some embodiments, the use of an active portion of a presently disclosed algal derived blue-shifted channelrhodopsin, such as but not limited to the rhodopsin domain, includes all or portions of the sequences described herein (and expression vectors comprising the same), and additionally contemplates the use of any nucleotide sequence encoding a contiguous an active portion of the presently disclosed algal derived blue-shifted channelrhodopsins, such as but not limited to the rhodopsin domain, open reading frame (ORF) that hybridizes to a complement of a channelrhodopsin or channelopsin sequence described herein under highly stringent conditions, e.g., hybridization to filter-bound DNA in 0.5 M NaHPO₄, 7% sodium dodecyl sulfate (SDS), 1 mM EDTA at 65° C., and washing in 0.1×SSC/0.1% SDS at 68° C. (“Current Protocols in Molecular Biology”, Vol. 1 and 2 (Ausubel et al., eds., Green Publishing Associates, Incorporated, and John Wiley & Sons, Incorporated, New York, N.Y., 1989)), and encodes a functionally equivalent channelrhodopsin (or active portion thereof, such as but not limited to the rhodopsin domain) gene product or the active portion thereof. Additionally contemplated is the use of any nucleotide sequence that hybridizes to the complement of a DNA sequence that encodes a channelrhodopsin amino acid sequence under moderately stringent conditions, e.g., washing in 0.2×SSC/0.1% SDS at 42° C. (“Current Protocols in Molecular Biology”, supra), yet still encodes a functionally equivalent channelrhodopsin product. Functional equivalents of channelrhodopsin include, but are not limited to, naturally occurring versions of channelrhodopsin present in other species (orthologs and homologs), and mutant versions of channelrhodopsin, whether naturally occurring or engineered (by site directed mutagenesis, gene shuffling, or directed evolution, as described in, for example, U.S. Pat. No. 5,837,458) or active portion thereof, such as but not limited to the rhodopsin domain. The disclosure also includes the use of degenerate nucleic acid variants (due to the redundancy of the genetic code) of the identified channelrhodopsin polynucleotide sequences.

Additionally contemplated is the use of polynucleotides encoding channelrhodopsin ORFs, or their functional equivalents, encoded by polynucleotide sequences that are about 99, 95, 90, or about 85 percent similar to the corresponding regions of the an algae derived channelrhodopsin sequences described herein (as measured by BLAST sequence comparison analysis using, for example, the University of Wisconsin GCG sequence analysis package (SEQUENCHER 3.0, Gene Codes Corporation, Ann Arbor, Mich.) using default parameters).

Transgenic Animals: The present disclosure provides methods and compositions for the creation and use of both human and non-human transgenic animals that carry an algae derived channelrhodopsin transgene in all their cells, as well as non-human transgenic animals that carry an algae derived channelrhodopsin transgene in some, but not all their cells, for example in certain neuronal cells. Human and non-human mammals of any species, including, but not limited to, mice, rats, rabbits, guinea pigs, pigs, micro-pigs, goats, and non-human primates, e.g., baboons, monkeys, and chimpanzees, can be used to generate transgenic animals carrying an algae derived channelrhodopsin polynucleotide (and/or expressing an algae derived polypeptide) may be integrated as a single transgene or in concatamers, e.g., head-to-head or head-to-tail tandems. An algae derived channelrhodopsin transgene may also be selectively introduced into and activated in a particular cell-type (see, e.g., Lakso et al., Proc. Natl. Acad. Sci. USA 89:6232-6236, 1992). The regulatory sequences required for such a cell-type specific activation will depend upon the particular cell-type of interest, and will be apparent to those of skill in the art.

Should it be desired that an algae derived channelrhodopsin, or fragment thereof, transgene be integrated into the chromosomal site of the endogenous copy of the mammalian channelrhodopsin gene, gene targeting is generally preferred. Briefly, when such a technique is to be utilized, vectors containing some nucleotide sequences homologous to the endogenous channelrhodopsin gene are designed for the purpose of integrating, via homologous recombination with chromosomal sequences, into and disrupting the function of the endogenous channelrhodopsin gene (i.e., “knock-out” animals). In this way, the expression of the endogenous channelrhodopsin gene may also be eliminated by inserting non-functional sequences into the endogenous channelrhodopsin gene. The transgene may also be selectively introduced into a particular cell-type, thus inactivating the endogenous channelrhodopsin gene in only that cell-type (see, e.g., Gu et al., Science 265:103-106, 1994). The regulatory sequences required for such a cell-type specific inactivation will depend upon the particular cell-type of interest, and will be apparent to those of skill in the art.

Any technique known in the art may be used to introduce a channelrhodopsin, or fragment thereof, transgene into animals to produce the founder lines of transgenic animals. Such techniques include, but are not limited to: pronuclear microinjection (U.S. Pat. No. 4,873,191); retrovirus-mediated gene transfer into germ lines (van der Putten et al., Proc. Natl. Acad. Sci. USA 82:6148-6152, 1985); gene targeting in embryonic stem cells (Thompson et al., Cell 56:313-321, 1989); electroporation of embryos (Lo, Mol. Cell. Biol. 3:1803-1814, 1983); sperm-mediated gene transfer (Lavitrano et al., Cell 57:717-723, 1989); and positive-negative selection, as described in U.S. Pat. No. 5,464,764. For a review of such techniques, see, e.g., Gordon, Int. Rev. Cytol. 115:171-229, 1989.

Once transgenic animals have been generated, the expression of the recombinant channelrhodopsin gene, or fragment thereof, may be assayed utilizing standard techniques. Initial screening may be accomplished by Southern blot analysis or PCR techniques to analyze animal tissues to assay whether integration of the channelrhodopsin transgene has taken place. The level of mRNA expression of the channelrhodopsin transgene in the tissues of the transgenic animals may also be assessed using techniques that include, but are not limited to, Northern blot analysis of cell-type samples obtained from the animal, in situ hybridization analysis, and RT-PCR. Samples of an algae derived channelrhodopsin-expressing tissue can also be evaluated immunocytochemically using antibodies selective for the channelrhodopsin transgene product.

In certain embodiments, the invention concerns isolated nucleic acid segments and recombinant vectors which encode a protein or peptide that includes within its amino acid sequence an amino acid sequence of a channelrhodopsin or a functional portions or variant thereof, such as those identified and cloned: PsChR1 (SEQ ID NOS: 1-3). In some embodiments, a portion of a channelrhodopsin and has relatively few amino acids which are not identical to, or a biologically functional equivalent of, the amino acids of the full-length channelrhodopsin. The term “functional equivalent” is well understood in the art. Accordingly, sequences which have between about 70% and about 80%; or more preferably, between about 85% and about 90%; or even more preferably, between about 90 and 95% and about 99%; of amino acids which are identical or functionally equivalent to the amino acids of the identified and cloned: PsChR (SEQ ID NOS: 1 and 3).

The nucleic acid segments of the present invention, regardless of the length of the coding sequence itself, may be combined with other sequences, such as promoters, polyadenylation signals, additional restriction enzyme sites, multiple cloning sites, other coding segments, and the like, such that their overall length may vary considerably. It is therefore contemplated that a nucleic acid fragment of almost any length may be employed, with the total length preferably being limited by the ease of preparation and use in the intended recombinant DNA protocol. For example, nucleic acid fragments may be prepared which include a short stretch complementary to nucleic acids that encode the polypeptides of SEQ ID NOS: 1 and 3, such as about 10 to 15 or 20, 30, or 40 or so nucleotides, and which are up to 2000 or so base pairs in length. DNA segments with total lengths of about 8000, 7000, 6000, 5000, 4000, 3000, 2000, 1000, 500, 200, 100 and about 50 base pairs in length are also contemplated to be useful.

In some embodiments, isolated nucleic acids that encode the amino acids of a channelrhodopsin or fragment thereof and recombinant vectors incorporating nucleic acid sequences which encode a channelrhodopsin protein or peptide and that includes within its amino acid sequence an amino acid sequence in accordance with SEQ ID NOS: 1 and 3. In some embodiments, a purified nucleic acid segment that encodes a protein that encodes a channelrhodopsin or fragment thereof, the recombinant vector may be further defined as an expression vector comprising a promoter operatively linked to said channelrhodopsin-encoding nucleic acid segment.

In additional embodiments, is a host cell, made recombinant with a recombinant vector comprising channelrhodopsin-encoding nucleic acid segments. The recombinant host cell may be a prokaryotic cell or a eukaryotic cell. As used herein, the term “engineered” or “recombinant” cell is intended to refer to a cell into which a recombinant gene, such as a gene encoding a channelrhodopsin, has been introduced. Therefore, engineered cells are distinguishable from naturally occurring cells which do not contain a recombinantly introduced gene. Engineered cells are thus cells having a gene or genes introduced through the hand of man. Recombinantly introduced genes will either be in the form of a copy of a genomic gene or a cDNA gene, or will include genes positioned adjacent to a promoter not naturally associated with the particular introduced gene. In some embodiments, nucleic acid molecules having sequence regions consisting of contiguous nucleotide stretches of about 14, 15-20, 30, 40, 50, or even of about 100 to about 200 nucleotides or so, identical or complementary to the channelrhodopsin-encoding nucleic acid sequences.

Transgene Based Therapies: The nucleic acids sequences that encode an active portion of the presently disclosed blue-shifted channelrhodopsins, include but are not limited to the nucleic acid sequences that encode the rhodopsin domains identified in SEQ ID NOS: 1 and 3 or the rhodopsin domain sequences of SEQ ID NO: 2.

In certain embodiments the presently disclosed compositions and are used to improve optogenetic techniques and applications as well as can be used to aid in diagnosis, prevention, and/or treatment of neurologic disorders, such as but not limited to Parkinson's disease, as well as for ocular disorders.

In some embodiments, methods and compositions are used to identify and characterize multiple channelrhodopsins derived from algae. The cloning and expression of the rhodopsin domain of the channelrhodopsins and expression in mammalian cells demonstrates that these channelrhodopsins have improved characteristics that can be used for optogenetic applications as well as therapeutic agents.

For example, a disclosed method and composition may be used in, among other things, retinal gene therapy for mammals (as described in, among others, U.S. Pat. Nos. 5,827,702, 7,824,869 and US Patent Publication Number 20100015095 as well as in WIPO publications WO 2000/15822 and WO 1998/48097). A genetically engineered ocular cell is produced by contacting the cell with an exogenous nucleic acid under conditions in which the exogenous nucleic acid is taken up by the cell for expression. The exogenous nucleic acid is described as a retrovirus, an adenovirus, an adeno-associated virus or a plasmid. Retinal gene transfer of a reporter gene, green fluorescent protein (GFP), using a recombinant adeno-associated virus (rAAV) was demonstrated in normal primates (Bennett, J et al. 1999 Proc. Natl. Acad. Sci. USA 96, 9920-25). The rescue of photoreceptors using gene therapy in a model of rapid degeneration of photoreceptors using mutations of the RP65 gene and replacement therapy with the normal gene to replace or supplant the mutant gene (See, for example, US Patent Publication 2004/0022766) has been used to treat a naturally-occurring dog model of severe disease of retinal degenerations—the RPE65 mutant dog, which is analogous to human LCA. By expressing photosensitive membrane-channels or molecules in surviving retinal neurons of the diseased retina by viral based gene therapy method, the present invention may produce permanent treatment of the vision loss or blindness with high spatial and temporal resolution for the restored vision.

In some embodiments, introduction and expression of channelrhodopsins, such as those described herein, inocular neuronal cells, for example, impart light sensitivity to such retinas and restoring one or more aspects of visual responses and functional vision to a subject suffering from such degeneration. By restoring light sensitivity to a retina lacking this capacity, due to disease, a mechanism for the most basic light-responses that are required for vision is provided. In some embodiments, the functional domains of channelrhodopsins, such as PsChR may be used to restore light sensitivity to the retinas that have undergone rod and cone degeneration by expressing the channelrhodopsin in inner retinal neurons in vivo. In some embodiments these channelrhodopsins may be introduced using techniques that include, but are not limited to, retinal implants, cortical implants, lateral geniculate nucleus implants, or optic nerve implants

In some embodiments, the blue-shifted channelrhodopsins are inserted into the retinal neurons that survived after the rods and cones have died in an area or portion of the retina of a subject, using the transfer of nucleic acids, alone or within an expression vector. Such expression vectors may be constructed, for example, by introduction of the desired nucleic acid sequence into a virus system known to be of use for gene therapy applications, such as, but not limited to, AAV, retroviruses and alike.

In some embodiments the blue-shifted channelrhodopsins may be inserted into retinal interneurons. These cells then can become light sensitive and send signals via the optic nerve and higher order visual pathways to the visual cortex where visual perception occurs, as has been demonstrated electrophysiologicly in mice. In some embodiments, among other routes, intravitreal and/or subretinal injections may be used to deliver channelrhodopsin molecules or virus vectors expressing the same.

In some embodiments, the active portion of the presently disclosed algal derived blue-shifted channelrhodopsins, such as but not limited to the rhodopsin domain of these channelrhodopsins, can be used to restore light sensitivity to a retina, by delivering to retinal neurons a nucleic acid expression vector that encodes algal derived blue-shifted channelrhodopsins (such as but not limited to the rhodopsin domain of these channelrhodopsins) that is expressible in the neurons, which vector comprises an open reading frame encoding the rhodopsin, and operatively linked thereto, a promoter sequence, and optionally, transcriptional regulatory sequences; and expressing the vector in the neurons, thereby restoring light sensitivity.

In certain embodiments the channel rhodopsin can be algal derived blue-shifted channelrhodopsins such as, but not limited to functional domains of channelrhodopsins, such as PsChR or a biologically active fragment or conservative amino acid substitution variant thereof, such as but not limited to the rhodopsin domain. The vector system may be recombinant AAV, the promoter may be a constitutive promoter such as, but not limited to, a CMV promoter or a hybrid CMV enhancer/chicken β-actin promoter (CAG).

The following Examples section provides further details regarding examples of various embodiments. It should be appreciated by those of skill in the art that the techniques disclosed in the examples that follow represent techniques and/or compositions discovered by the inventors to function well. However, those of skill in the art should, in light of the present disclosure, appreciate that many changes can be made in the specific embodiments which are disclosed and still obtain a like or similar result without departing from the spirit and scope of the invention. These examples are illustrations of the methods and systems described herein and are not intended to limit the scope of the invention. Non-limiting examples of such include, but are not limited to those presented below.

### EXAMPLES

### Characterization of Channelrhodopsins: Light-Induced Electrical Responses

### Example 1.1

### Source and Growth of Algae

Platymonas (Tetraselmis) subcordiformis and Dunaliella salina were obtained from the UTEX Culture Collection of Algae (#71 and #LB1644, respectively) and grown in modified artificial seawater medium A (McLachlan, J. 1960. The culture of Dunaliella tertiolecta Butcher: a euryhaline organism. Can. J. Microbiol. 6: 367-379) and Johnson's medium (Haghjou, M. M., M. Shariati, and N. Smirnoff. 2009. The effect of acute high light and low temperature stresses on the ascorbate-glutathione cycle and superoxide dismutase activity in two Dunaliella salina strains. Physiol. Plant.135: 272-280), respectively, under a 16/8 light/dark cycle (light: 2000 and 3000 lux, respectively).

### Example 1.2

### Generation of CDNA and Cloning

P. subcordiformis and D. salina: Total RNA was extracted with Trizol reagent (Invitrogen, Carlsbad, Calif.). First strand cDNA was synthesized with the SMARTer RACE cDNA amplification kit (Clontech Laboratories, Mountain View, Calif.). Degenerate primers for P. subcordiformis were designed according to the conserved regions of previously known channelopsins. First PCR (polymerase chain reaction) was carried out with primers TGCGGNTGGGAGGAGRTNTA (SEQ ID NO: 21) and KCCCTCRGKBCCCARBAGGAAS (SEQ ID NO: 22), after which the product mixture was subjected to another round of PCR with the nested forward primer TACGSNGANTGGYTNCTNACNTGCCC (SEQ ID NO: 23). PCR products of the appropriate size were gel-purified, cloned into the TOPO TA cloning vector (Invitrogen, Grand Island, N.Y.) and sequenced. For the fragment that showed homology with channelopsins, 3′ and 5′ RACE (rapid amplification of cDNA ends) was performed using the SMARTer RACE cDNA amplification kit. Overlapping RACE fragments were joined by fusion PCR to obtain full-length cDNA, which was cloned into the TOPO vector and sequenced. The 7TM domain of D. salina channelopsin (SEQ ID NO: 4) was cloned using the reverse primer GAGGGACGCGCGTGTTTGAGTGCCG (SEQ ID NO: 24) designed according to the sequence information.

The archaeorhodopsin-3 (AR-3) coding sequence was cloned into the E. coli expression vector pET28b (+) under control of an IPTG-inducible promoter, and into the mammalian expression vector (see below). E. coli strain BL21(DE3) was transformed with the AR-3− carrying expression vector, grown till OD₆₀₀=0.4 and induced by IPTG in the presence of 5 μM of all-trans retinal. The culture was harvested after 4 h, washed in distilled water and transferred to low-ionic-strength medium consisting of (in mM) 1.5 NaCl, 0.15 CaCl₂, 0.15 MgCl₂, and 5 Tris, pH 7.2. Photocurrents in E. coli cell suspensions were evoked by a Vibrant HE 355 II tunable laser (5 ns, 35 mJ; Opotek, Carlsbad, Calif.) with flashes set to the wavelengths of maximum absorption of AR-3 and its mutant applied along the direction between two platinum electrodes and recorded as described previously (Sineshchekov, O. A., and J. L. Spudich. 2004. Light-induced intramolecular charge movements in microbial rhodopsins in intact E. coli cells. Photochem. Photobiol. Sci. 3:548-554).

The mammalian expression vector pcDNA3.1/VcChR1-EYFP that contains a human-codon-optimized version of the 7TM domain of VcChR1 flanked with Bam HI and Not I sites (Optogenetics Resource Center. http://www.stanford.edu/group/dlab/optogenetics/) (Karl Deisseroth, Stanford University, Calif., USA) was utilized. For expression of other channelrhodopsins, the VcChR1 sequence was replaced with algal cDNA fragments encoding the 7TM domains of channelrhodopsins CaChR1 (residues 1-352, SEQ ID NO: 5), DsChR1 (residues 1-365, SEQ ID NO: 4), MvChR1 (residues 1-331, SEQ ID NO: 6), CrChR2 (residues 1-309, SEQ ID NO: 7), and PsChR (residues 1-326, SEQ ID NO: 1). Mutations were introduced using a QuikChange XL site-directed mutagenesis kit (Agilent Technologies, Santa Clara, Calif.) and verified by DNA sequencing.

HEK293 cells were transfected using the TransPass COS/293 transfection reagent (New England Biolabs, Ipswich, Mass.). All-trans retinal (Sigma, St. Louis, Mo.) was added as a stock solution in ethanol at a final concentration of 5 μM. Measurements were performed 48-72 h after transfection with an Axopatch 200B amplifier (Molecular Devices, Union City, Calif.) with a 10-kHz filter. The signals were digitized with a Digidata 1440A using pClamp 10 software (both from Molecular Devices, Eugene, Oreg.) at a sampling rate of 4 μs/point. Patch pipettes with resistances of 2-5 MΩ were fabricated from borosilicate glass and filled with a solution containing (in mM) 126 KCl, 2 MgCl₂, 0.5 CaCl₂, 5 EGTA, and 25 HEPES, pH 7.4, unless otherwise indicated. The bath solution contained (in mM) 150 NaCl, 1.8 CaCl₂, 1 MgCl₂, 5 glucose, and 10 HEPES, pH 7.4, unless otherwise indicated. Flash excitation (pulsewidth 6 ns, energy 12 mJ) was via a Minilite Nd:YAG laser (Continuum, Santa Clara, Calif.) at wavelength 532 nm and an interval of 2 s between flashes, unless otherwise indicated. A laser artifact measured with a blocked optical path was digitally subtracted from the recorded traces. For further analysis, the signals were logarithmically averaged with a custom-created computer algorithm. Numerical data in the text are presented as the mean±SE.

A Pichia clone that expresses the 7TM domain of wild-type CaChR1 was obtained as described in US patent publication US20130066047 and Hou et al, (Hou, S. Y., Govorunova, E. G., Ntefidou, M., Lane, C. E., Spudich, E. N., Sineshchekov, O. A., and Spudich, J. L. 2012 Diversity of Chlamydomonas channelrhodopsins. Photochem. Photobiol. 88, 119-128) and a similar procedure was used to select clones that express the CaChR1_E169Q and CaChR1_D299N mutants. Cells were grown in buffered minimal methanol yeast medium, and expression was induced by the addition of 0.5% methanol every 24 h in the presence of 5 μM all-trans retinal. Cells were grown for two days, harvested by low-speed centrifugation, and disrupted by a bead beater. Membrane fragments were collected by centrifugation for 1 h at 48,000 rpm. The proteins were partially purified on a Ni-NTA agarose column (Qiagen, Hilden, Germany) after solubilization by incubation with 2% dodecyl maltoside for 1 h.

Absorption changes of Pichia-expressed pigments were induced using a Surelight I Nd-YAG laser flash (532 nm, 6 ns, 40 mJ; Continuum, Santa Clara, Calif.) and measured with a laboratory-constructed cross-beam measuring system, as described previously (Wang, W.-W., Sineshchekov, O. A., Spudich, E. N., and Spudich, J. L. 2003. Spectroscopic and photochemical characterization of a deep ocean proteorhodopsin. J. Biol. Chem. 278, 33985-33991). For calculation of the relative yields of the M-like intermediate formation in wild-type CaChR1 and its mutants, flash-induced absorbance changes at the maximal absorption of the M intermediate were normalized to the pigment concentration (maximal absorption of the unphotolysed state) and corrected for the relative absorption at the excitation wavelength (532 nm) according to fluence response curves. PsChR demonstrated a dramatically increased level of expression and, correspondingly, the amplitude of channel currents generated in animal cells upon the addition of exogenous retinal than did CrChR. This increase was even larger than that seen with CrChR2. Therefore, the intact mammalian brain may have enough endogenous retinal to reconstitute fully functional PsChR.

### Example 1.3

### Testing the Measuring System with a Proton Pump

The time resolution of whole-cell patch-clamp measurements is limited by the series resistance and the membrane capacitance of the cell. To estimate the degree of signal integration and facilitate interpretation of fast currents generated in HEK cells by channelrhodopsins, signals from the proton pump archaerhodopsin-3 (AR-3, or Arch (Chow B Y, Han X, Dobry A S, Qian X, Chuong A S, Li M, Henninger M A, Belfort G M, Lin Y, Monahan P E, Boyden E S. 2010. High-performance genetically targetable optical neural silencing by light-driven proton pumps. Nature. 463:98-102; Husson S J, Liewald J F, Schultheis C, Stirman J N, Lu H, Gottschalk A. 2012. Microbial light-activatable proton pumps as neuronal inhibitors to functionally dissect neuronal networks in C. elegans. PLoS ONE. 7:e40937) were first examined. Unlike most channelrhodopsins, this protein can also be produced in E. coli cells, in suspensions of which intramolecular charge movements associated with rhodopsin photocycling can be recorded with time resolution at least an order of magnitude better than that observed for whole-cell patch-clamp recording (Sineshchekov, O. A., and J. L. Spudich. 2004. Light-induced intramolecular charge movements in microbial rhodopsins in intact E. coli cells. Photochem. Photobiol. Sci. 3:548-554). The outward proton-transfer current recorded from wild-type AR-3 in E. coli suspensions could also be detected by whole-cell patch clamp in HEK cells (FIG. 1, upper solid curve). However, in the latter system, its peak was almost 10-fold later than when measured in E. coli suspensions due to integration by the measuring circuit.

Initial stages of the charge transfer in rhodopsins associated with the formation of K and L intermediates of the photocycle occur on a submicrosecond timescale and are not resolved even when recorded in E. coli suspensions. The initial negative current overlaped to a great extent the subsequent fast positive current associated with the formation of the M intermediate, and their kinetics and amplitudes influence each other (FIG. 1, upper dashed curve). When the positive current was suppressed by neutralization of the primary proton acceptor Asp⁹⁵ in the AR-3_D95N mutant, both techniques registered a large, unresolved negative current (FIG. 1, lower), which was not resolved in HEK cells expressing the wild-type. In E. coli, where this signal was also visible in the wild-type, its apparent peak shifted to longer times in the mutant, as expected due to suppression of the subsequent positive peak. These results demonstrated that both the initial negative and subsequent fast positive currents generated by microbial rhodopsins can be detected and semiquantitatively analyzed in HEK cells.

### Example 1.4

### Outward Proton Transfer

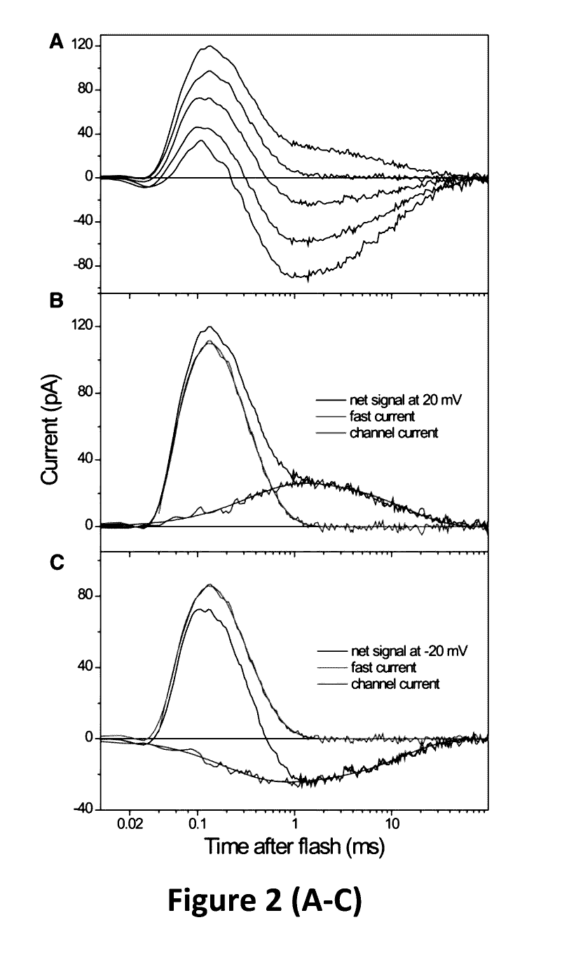

CaChR1 from Chlamydomonas augustae demonstrated typical channel activity when expressed in HEK cells. Electrical signals generated by CaChR1 in response to a laser flash showed complex kinetics (FIG. 2). At negative holding voltages (Vₕ), a small negative peak appeared at ˜25 μs after the flash, followed by a larger positive peak at 130-140 μs and a negative peak of channel current at 1-2 ms. The relative contributions of all currents to the net signal kinetics were independent of the flash intensity and the time interval between successive flashes (FIG. 1), indicating that they were initiated by excitation of the unphotolyzed pigment rather than subsequent photocycle intermediates. At the reversal potential (Vᵣ) for passive channel current (˜0 mV), the trace was dominated by the fast positive current. Net signal traces recorded at other holding voltages (FIG. 2, B and C, black lines) could be fit with superposition of a properly scaled fast positive current (FIG. 2, B and C, red lines) and a slow passive channel current (FIG. 2, B and C, blue lines).

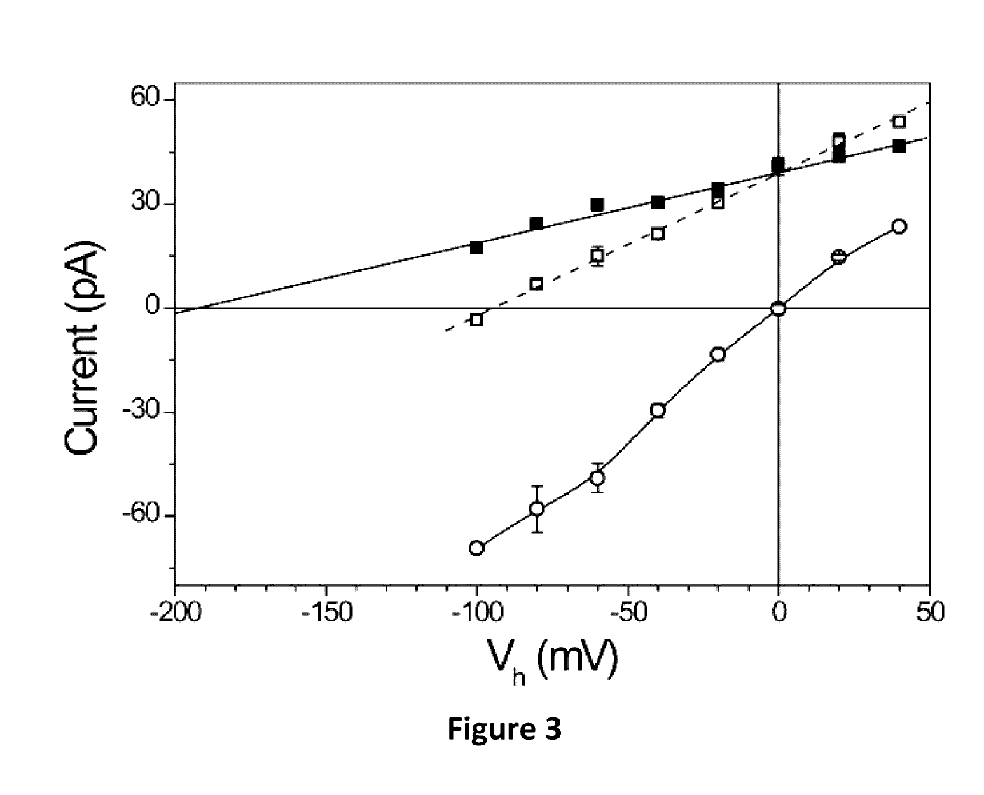

The voltage dependence of the fast positive current was evident from the presence of a distinct peak in the differential signals calculated by subtraction of traces recorded at different Vₕ (FIG. 3). The current-voltage relationship (I-V curve) of the current averaged over the time of the fast positive phase was linear and crossed the zero line at ˜−95 mV, i.e., it was dramatically shifted to negative voltages with respect to the reversal potential of channel current (FIG. 3, open squares and circles, respectively). When the amplitude of the fast positive current was corrected for the contribution of channel current, extrapolation of its voltage dependence predicted its complete inhibition at ˜−200 mV (FIG. 3, solid squares).

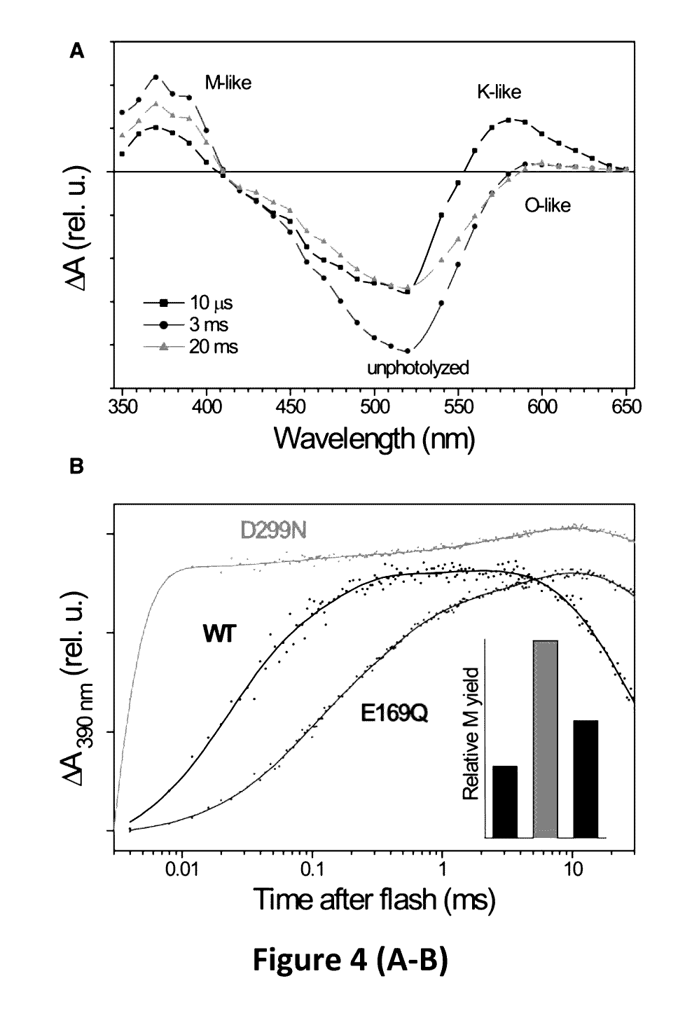

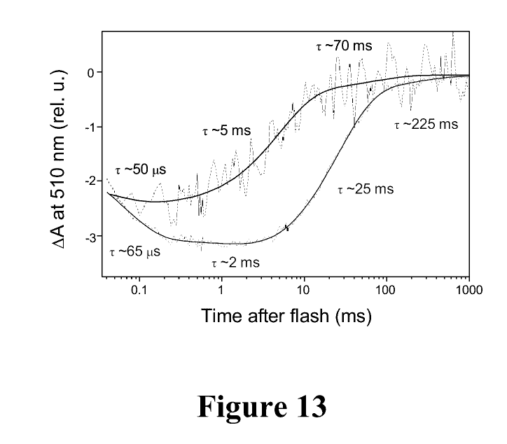

Flash photolysis measurements of CaChR1 from Pichia revealed a photocycle typical of microbial rhodopsins with K-, M-, and O-like intermediates (FIG. 4A). Two major components of the M-like intermediate formation in CaChR1 had the time constants ˜20 and 110 μs and almost equal amplitudes (FIG. 4B, black line). These values are only slightly slower than those reported earlier for the appearance of the M-like intermediate in the photocycle of ChR2 from C. reinhardtii (Verhoefen M K, Bamann C, Blöcher R, Förster U, Bamberg E, Wachtveitl J. 2010. The photocycle of channelrhodopsin-2: ultrafast reaction dynamics and subsequent reaction steps. ChemPhysChem. 11:3113-3122). However, instead of a fast decay observed in CrChR2 on the timescale of several milliseconds, the concentration of the M-like intermediate of CaChR1 remained stable or even slightly increased with t˜3 ms (FIG. 4B, black line) and decayed very slowly with time constants of 32 and 200 ms (data not shown). The fast components of the M formation in the photocycle of detergent-purified CaChR1, as probed by flash photolysis, roughly correlated with the time domain of the fast positive electrical current. An exact temporal correlation between the fast electrical and optical components could not be expected because on the one hand, the time resolution of patch-clamp current measurements was limited, and on the other, the photocycle rate of detergent-solubilized CaChR1 was around three times slower than that of the pigment in intact Pichia membranes (FIG. 13).

Neither the amplitude nor voltage dependence of the fast positive current changed upon a 100-fold decrease in the Na⁺ concentration in the bath, whereas the amplitude of the channel current decreased by ˜28% and its Vᵣ shifted ˜12 mV to negative values, indicating that predominantly proton-selective CaChR1 is also permeable for Na⁺. The fast positive current also did not change when K⁺ in the pipette solution was substituted with Na⁺, which rules out a contribution of a passive K⁺ efflux to the current.

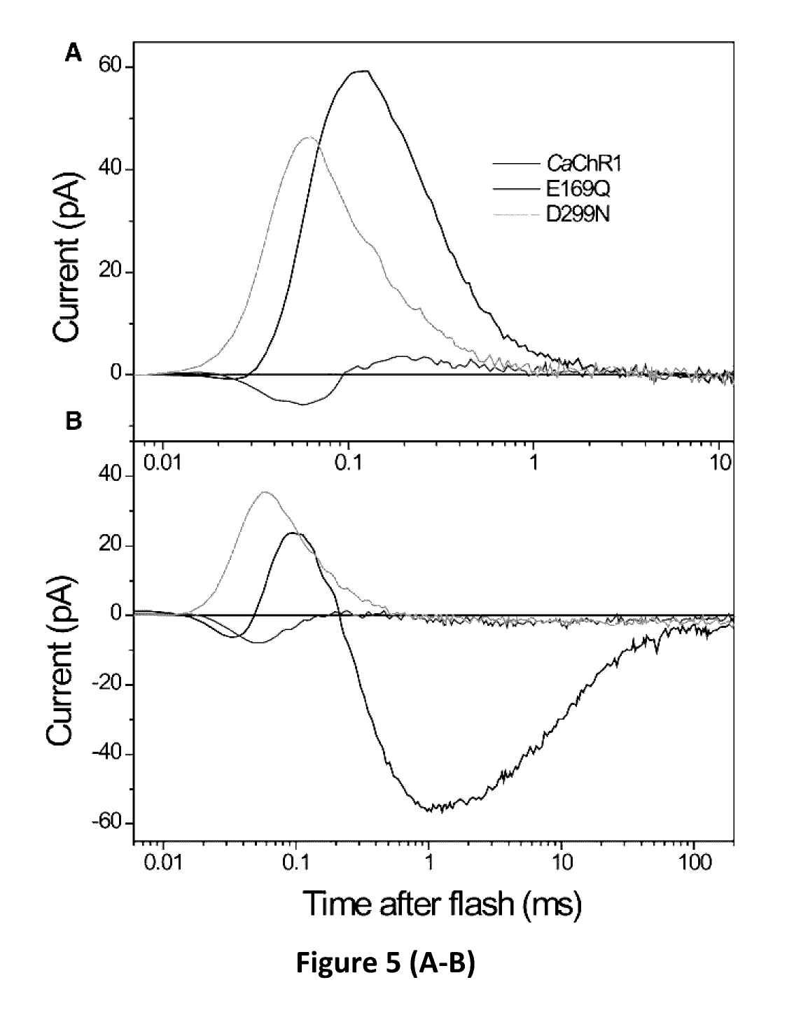

Weak dependence of the fast positive current on the membrane potential and cation composition of the media, as well as its semiquantitative correlation with the time course of the M-intermediate formation led to the conclusion that it reflects proton transfer from the Schiff base to an outwardly located acceptor(s). This interpretation was confirmed in experiments with site-specific mutations. Neutralization of Glu¹⁶⁹, which is homologous to the primary proton acceptor Asp⁸⁵ in BR, led to a dramatic, almost 20-fold decrease in the amplitude of the fast positive current (BJ FIG. 5A, red line). The remaining positive current was slow, with a peak time of ˜220 μs. In the E169Q mutant, the unresolved fast negative signal related to early stages of the photocycle became visible, whereas in the wild-type CaChR1 it was apparently canceled by the fast outwardly directed proton movement. Remarkably, channel current in this mutant was suppressed to approximately the same degree as the fast positive current (BJ FIG. 5B), so that their ratio remained almost equal to that in the wild-type, 1.19±0.46 (n ¼ 5 cells) and 1.24±0.32 (n ¼ 7 cells), respectively.

Neutralization of Asp²⁹⁹, which is homologous to the second aspartate of the complex counterion of the Schiff base in BR (Asp²¹²), produced different changes in photo-currents. The amplitude of the fast positive current measured at the reversal potential for channel current was only slightly reduced, and at −60 mV was even higher than that in the wild-type at the same voltage (FIG. 5, green lines). Moreover, the current in the D299N mutant accelerated relative to that in wild-type, so that its peak time decreased from ˜125 μs to ˜50 μs.

Despite only a slight reduction of the Schiff-base deprotonation current in the CaChR1_D299N mutant, its channel current was suppressed even more than that of the CaChR1_E169Q mutant. The ratio of the channel current measured at −60 mV to the fast positive current measured at the Vᵣ for channel current was ˜25-fold smaller in the CaChR1_D299N mutant (0.048±0.007, n ¼ 8) than in the wild-type or in CaChR1_E169Q. The remaining small channel current in CaChR1_D299N was dramatically slower compared to that in the wild-type. In particular, the time constant of the fast component of channel closing increased from 10 ms in the wild-type to ˜400 ms in the mutant.

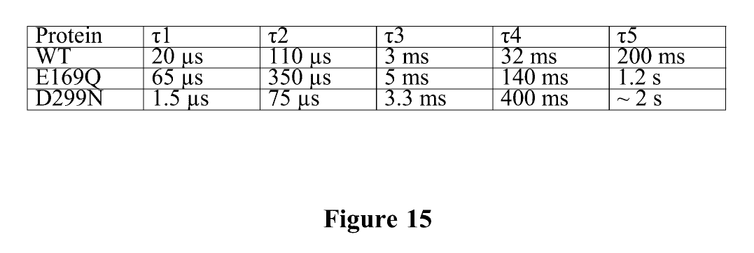

CaChR1_E169Q and CaChR1_D299N were expressed in Pichia, and their photocycles were analyzed by flash photolysis. Accumulation of an M intermediate was found in both mutants (FIG. 4B). The yields of M formation in the mutants were even higher than in the wild-type (2.75- and 1.63-fold, respectively (FIG. 4B, inset)) due to a much slower rate of its disappearance. In full agreement with the results of electrical measurements, the M intermediate formation was slowed down in the CaChR1_E169Q mutant and accelerated in the CaChR1_D299N mutant compared to its rate in the wild-type (FIG. 4B). The half-rise time of the M intermediate formation in these mutants was around seven times larger and around six times smaller, respectively, relative to that observed in the wild-type. In all three proteins, the rise of the M intermediate formation could be fit with three exponentials and the decay with two exponentials (see the corresponding time constants in Table of FIG. 15).

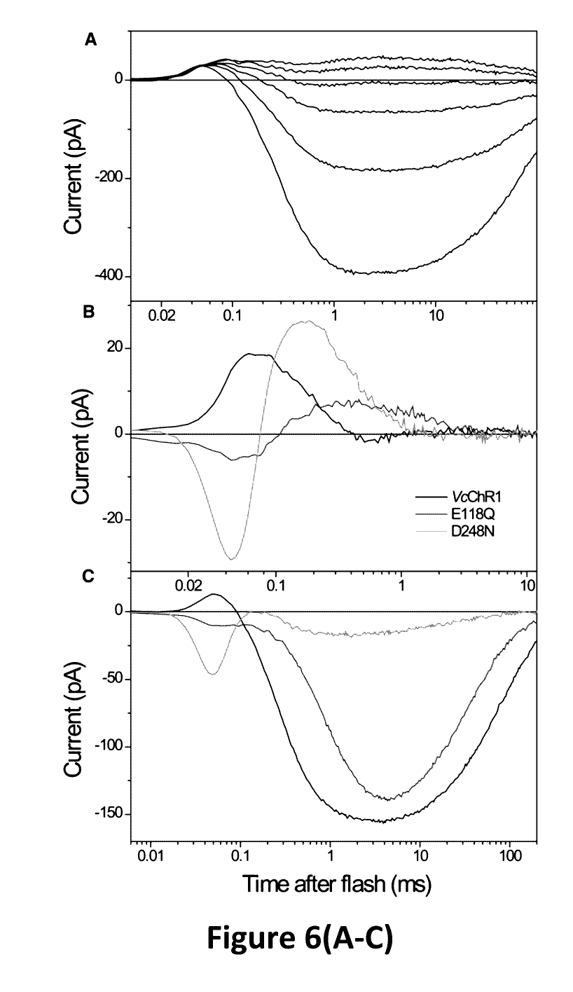

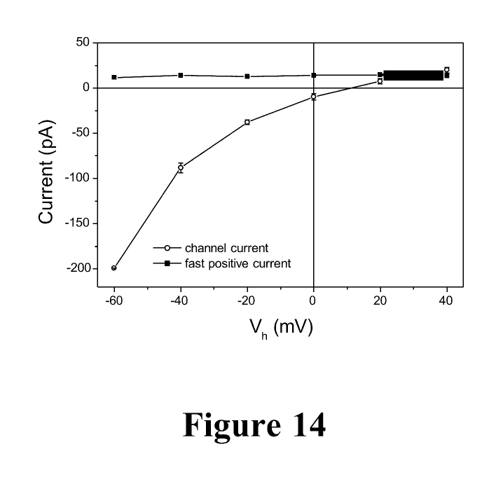

A fast current, similar to that produced by CaChR1, was also generated by channelrhodopsin from Volvox carteri (VcChR1). This current was significantly faster in VcChR1 (peak time, ˜80 μs Vᵣ (FIG. 6)) than in CaChR1. It also demonstrated an even weaker dependence on the holding potential (FIG. 14). In a similar way, this initial component could not be resolved in the signals recorded from AR-3 expressed in HEK cells due to its cancellation by a subsequent fast positive current, although it was clearly visible when measured at a higher time resolution in E. coli suspensions (FIG. 1).

The Vᵣ for channel current in VcChR1 was more positive (−10 mV (FIG. 14)) than that in CaChR1, indicating a higher Na⁺/H⁺ permeability ratio. Signals generated by VcChR1 at Vᵣ for channel currents differed from those generated by CaChR1 in that they showed a small additional negative wave at ˜0.5 ms, which could not be eliminated by variation of Vₕ (FIG. 6B, black line).

Neutralization of either of the two residues homologous to those that form the Schiff-base counterion in BR had effects in VcChR1 qualitatively similar to those observed in CaChR1. Specifically, relative to the wild-type, the VcChR1_E118Q mutant (in which the residue corresponding to Asp⁸⁵ in BR was mutated) demonstrated a significantly smaller and slower positive current, whereas its channel current was only slightly reduced (FIG. 6, B and C, red lines). In contrast, mutation of Asp²⁴⁸ (corresponding to Asp²¹² in BR) did not diminish the positive current but greatly suppressed channel current (FIG. 6, B and C, green lines). In the D248N mutant, the ratio of the channel current amplitude at −60 mV to that of the positive current at Vᵣ was only 1.0±0.3 (n ¼ 3), as compared to 7.9±2.0 (n ¼ 5) in the wild-type and 19.2±3.4 (n ¼ 3) in the E118Q mutant. Remarkably, the reversal potential for the remaining channel current in the D248N mutant shifted to ˜0 mV, indicating that neutralization of this residue primarily suppresses cation conductance. These results indicated that in VcChR1 the residue corresponding to Asp⁸⁵ in BR serves as the primary proton acceptor.

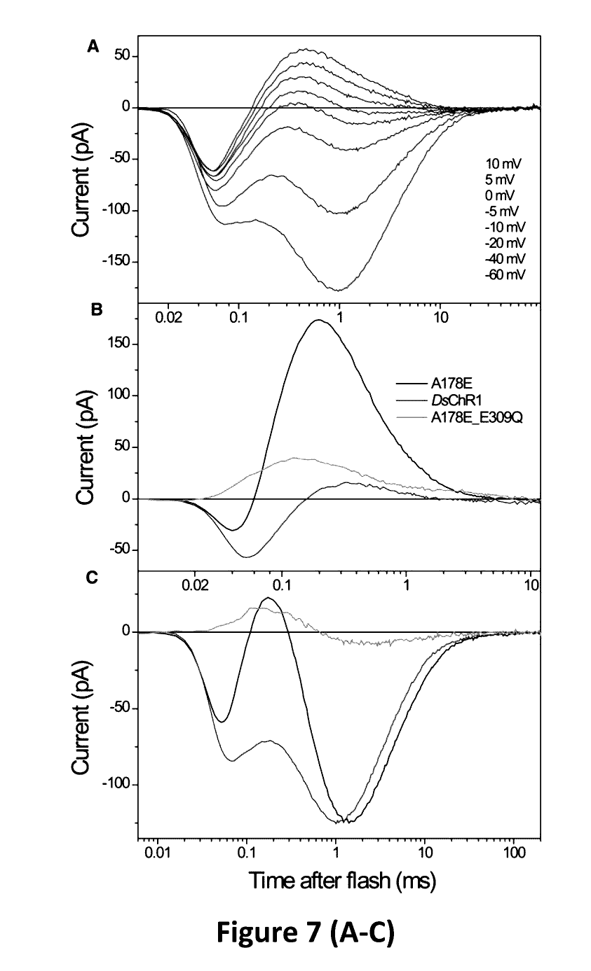

Except for DsChR1 from D. salina, all prior identified channelrhodopsins have contained a Glu residue in the position of the Schiff-base proton acceptor Asp⁸⁵ in BR, whereas in DsChR1 this site is occupied by alanine (Ala¹⁷⁸). Interestingly, the 7TM domain of DsChR1 (SEQ ID NO: 4) showed only 90% identity at the protein level with the previously reported sequence (Zhang F, Vierock J, Yizhar O, Fenno L E, Tsunoda S, Kianianmomeni A, Prigge M, Berndt A, Cushman J, Polle J, Magnuson J, Hegemann P, Deisseroth K. 2011.The microbial opsin family of optogenetic tools. Cell. 147:1446-1457). Most differences were found in the N-terminal part of the protein, whereas Ala¹⁷⁸ and all other residues so far identified as functionally important were conserved. DsChR1 showed much larger amplitude of the fast unresolved negative current (FIG. 7A) compared to both wild-type pigments described above. Such large negative currents were also observed upon neutralization of the corresponding residues in the CaChR1_E169Q and VcChR1_E118Q mutants (FIGS. 5 and 6, respectively, red lines), and can be explained by the reduction of the amplitude and rate of the subsequent positive proton transfer current. In DsChR1, the apparent peak time of this current was 406±46 ms, n ¼ 6.

A DsChR1 mutant in which Ala¹⁷⁸ was replaced with Glu was generated and tested and the residue found in this position in all other known wild-type channelrhodopsins. In this mutant, the fast positive current dramatically increased and accelerated relative to that in the wild-type (FIG. 7B, black line), so that the signal became similar to those recorded from wild-type CaChR1 and VcChR1, which naturally contain Glu in the corresponding position. The A178E mutation of DsChR1 did not significantly affect channel currents, as compared to the wild-type FIG. 7C). A double mutant in which only the homolog of Asp⁸⁵ was present (DsChR1_A178E_E309Q) was also generated and tested. As in the case of the corresponding CaChR1 mutant, this protein showed accelerated and only moderately decreased outward proton transfer current (FIG. 7B, green line), whereas the ratio of channel current to proton transfer current was significantly (˜15-fold) diminished (FIG. 7C, green line). As in the case of VcChR1, neutralization of the homolog of Asp⁸⁵ in BR did not change the reversal potential of channel current in DsChR1, whereas neutralization of the homolog of Asp²¹² shifted it to negative values by >20 mV.

### Example 1.5

### No Detectable Outward Proton Transfer

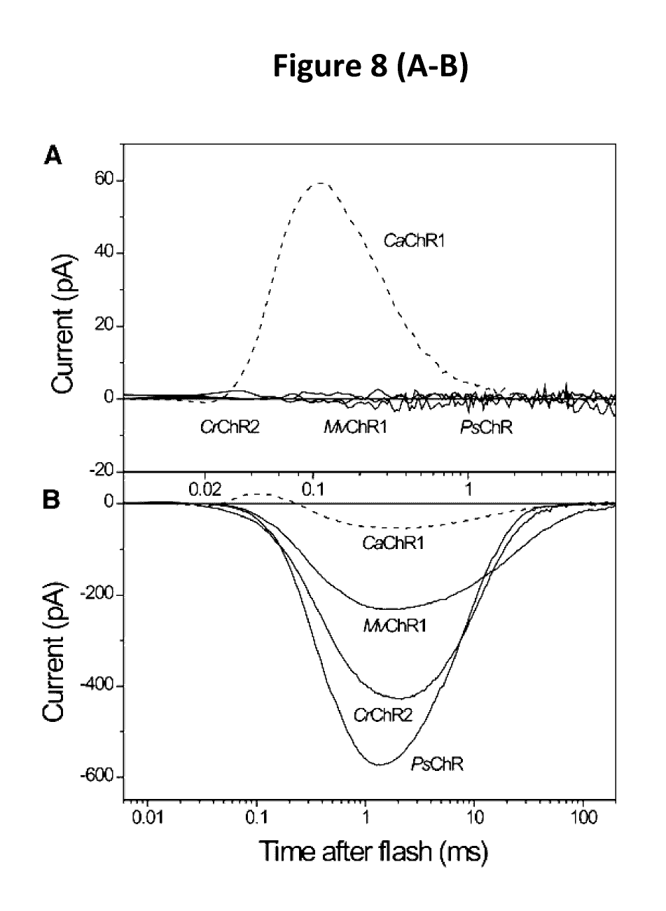

CrChR2 from C. reinhardtii is the best studied channelrhodopsin variant and the one most frequently used in optogenetic applications (Zhang F, Wang L P, Boyden E S, Deisseroth K. 2006. Channelrhodopsin-2 and optical control of excitable cells. Nat. Methods. 3:785-792). Flash photolysis studies of detergent-purified CrChR2 have established that the formation of its blue-shifted M intermediate occurs with time constants in the 10-100 ms range (Verhoefen M K, Bamann C, Blöcher R, Förster U, Bamberg E, Wachtveitl J. 2010. The photocycle of channelrhodopsin-2: ultrafast reaction dynamics and subsequent reaction steps. ChemPhysChem. 11:3113-3122; Govorunova, E. G., Spudich, E. N., Lane, C. E., Sineshchekov, O. A., and Spudich, J. L. 2011. New channelrhodopsin with a red-shifted spectrum and rapid kinetics from Mesostigma viride. mBio 2, e00115-00111), similar to that in CaChR1 (FIG. 4B). However, no electrical currents corresponding to an outward proton transfer from the Schiff base could be resolved in this time domain upon laser excitation of wild-type CrChR2 at the reversal potential for channel current (FIG. 8). Similarly, no fast currents could be detected at the reversal potential in two other highly efficient channelrhodopsins: MvChR1 from Mesostigma viride (Govorunova, et al., 2011, ibid), and PsChR, which we cloned from the marine alga Platymonas (Tetraselmis) subcordiformis (accession no. JX983143) (FIG. 8A).

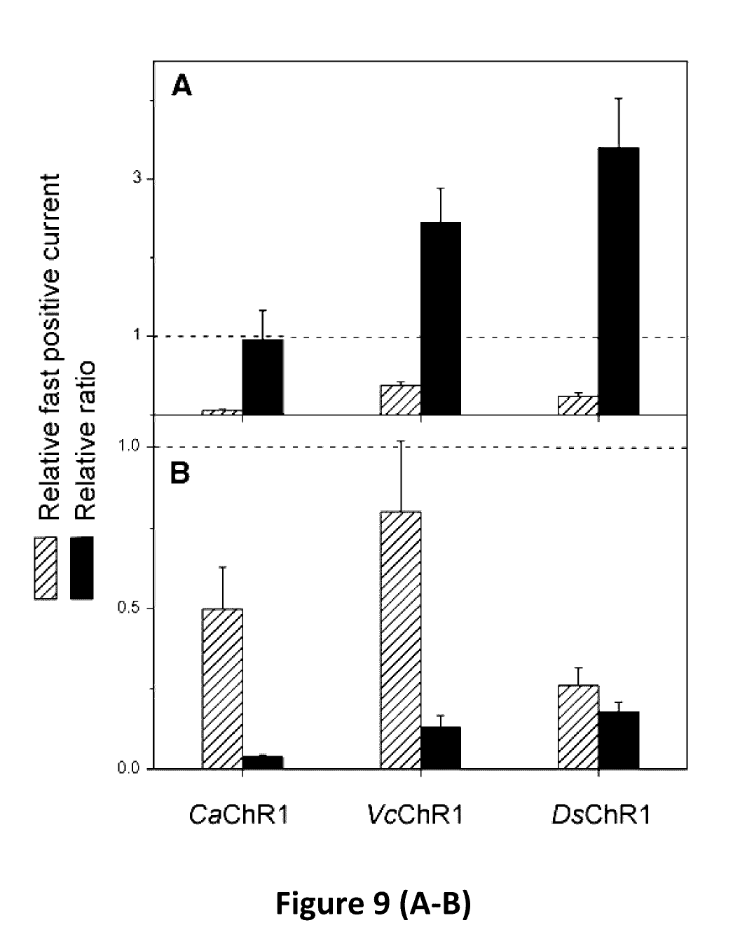

The reversal potentials for channel currents in these rhodopsins were positive (9.5, 15, and type (FIG. 9A). On the other hand, the residue in the position of Asp²¹² in BR is less important than the Asp⁸⁵ homolog for proton transfer in channelrhodopsins but is critical for channel opening. Neutralization of this residue induced only minor changes in the outward proton transfer currents but dramatically decreased the amplitude of channel currents (FIG. 9B). Moreover, the shifts in the reversal potentials of channel currents observed upon neutralization of the Asp²¹² homolog, but not the Asp⁸⁵ homolog, in VcChR1 and DsChR1 indicate that it primarily controls cation, rather than proton, conductance. In the second group of channelrhodopsins (CrChR2, MvChR1, and PsChR), no fast currents that reflect proton transfer from the Schiff base have been detected. The mean amplitude of channel currents generated by the newly cloned PsChR was actually larger than that generated by CrChR2 (FIG. 8B), which makes PsChR a strong candidate for optogenetic applications.

### Example 2

### Characterization of PsChR

### Example 2.1

### Photoelectric Currents

Photoelectric currents in Platymonas (Tetraselmis) subcordiformis cells were measured with the population assay described previously (Sineshchekov, O. A., Govorunova, E. G., Der, A., Keszthelyi, L., and Nultsch, W. 1992. Photoelectric responses in phototactic flagellated algae measured in cell suspension. J. Photochem. Photobiol. B: Biol. 13, 119-134). The method takes advantage of the directional sensitivity of the photoreceptor antenna complex in flagellates. Two platinum wires immersed in a cell suspension pick up an electrical current generated in response to a unilateral excitation flash from a Vibrant HE 35511 Tunable Laser (OPOTEK Inc., Carlsbad, Calif.) set at desired wavelengths. To decrease the current noise, cells from a 2-weeks culture were harvested and resuspended in the measuring medium of a lower ionic strength (0.5 mM CaCl2 and 40 mM NaCl). The signal was amplified by a low-noise current amplifier (Model 428, Keithley Instruments, Cleveland, Ohio) and digitized by a Digidata 1322A supported by pClamp 10 software (both Molecular Devices, Union City, Calif.).

### Example 2.2

### Whole-Cell Patch Clamp Measurements

Whole-cell patch clamp measurements in HEK293 cells. The mammalian expression vector that contained PsChR cDNA encoding the 7TM domain (amino acid residues 1-326) in frame with EYFP tag was generated as described previously (Sineshchekov, O. A., Govorunova, E. G., Wang, J., Li, H., and Spudich, J. L. 2013. Intramolecular proton transfer in channelrhodopsins. Biophys. J. 104, 807-817). HEK293 (human embryonic kidney) cells were transfected using the TransPass COS/293 transfection reagent (New England Biolabs, Ipswich, Mass.). All-trans-retinal (Sigma) was added as a stock solution in ethanol at the final concentration of 5 μM. Measurements were performed 48-72 h after transfection with an Axopatch 200B amplifier (Molecular Devices, Union City, Calif.) with a 2 kHz filter.

The signals were digitized with a Digidata 1440A using the pClamp 10.2 software (both from Molecular Devices) at the sampling rate 50 μs/point for noise analysis and 200 μs/point for other experiments. Fabrication of patch pipettes and contents of pipette and bath solutions were as before (Sineshchekov, et al., 2013, ibid). To test relative permeability for Na⁺ ions, Na⁺ in the bath solution was partially replaced with large monovalent cation N-methyl-D-glucamine (NMG⁺) that is not conducted by ChRs to a measurable degree (Nagel, et al., 2003). Light excitation was provided by a Polychrome IV light source (T.I.L.L. Photonics GMBH, Grafelfing, Germany) pulsed with a mechanical shutter (Uniblitz Model LS6, Vincent Associates, Rochester, N.Y.; half-opening time 0.5 ms).

The light intensity was attenuated with the built-in Polychrome system or with neutral density filters. Maximal quantum density at the focal plane of the 40× objective lens was ˜2×10²² photons×m⁻²×s⁻¹.

### Example 2.3

### Noise Analysis

An experimental procedure for stationary noise analysis of ChR-generated whole-cell currents was developed (modified from Feldbauer, K., Zimmermann, D., Pintschovius, V., Spitz, J., Bamann, C., and Bamberg, E. 2009. Channelrhodopsin-2 is a leaky proton pump. Proc. Natl. Acad. Sci. USA 106, 12317-12322). Current traces were recorded at −60 mV at room temperature in the dark and during a 25-s light pulse of intensity eliciting a half-maximal response. The plateau current was fit with a single exponential, and the fit signal was subtracted from the current trace (Cherny, V. V., Murphy, R., Sokolov, V., Levis, R. A., and DeCoursey, T. E. 2003. Properties of single voltage-gated proton channels in human eosinophils estimated by noise analysis and by direct measurement. J. Gen. Physiol. 121, 615-628). Power spectral densities were calculated from 5-s segments of the traces using pClamp software; ten individual spectra for dark and light conditions were averaged for each trace. The difference (light minus dark spectrum) was fit between 2 Hz and 1 kHz with a single Lorentzian function to determine the zero frequency asymptote and the corner frequency as described in Gray, P. 1994. (Analysis of whole cell currents to estimate the kinetics and amplitude of underlying unitary events: relaxation and “noise” analysis. in Microelectrode Techniques: The Plymouth Workshop Handbook (Ogden, D. C. ed.), Company of Biologists, Cambridge, UK. pp 189-207).

The theory of stationary noise analysis is based on the assumption that the channel stochastically alternates between a closed and an open state (Gray, 1994, ibid). The spectral density of resultant current fluctuations is described by a Lorentzian function:

$$S ( f ) = \frac{S ( 0 )}{1 + {( \frac{f}{{f}_{c}} )}^{2}}$$

where S(f) is the spectral density, S(0) is the zero asymptote (spectral density at 0 Hz), f is the frequency, and f_{c} is the corner frequency.

The unitary conductance (γ) is estimated from the parameters of this function using the amplitude of the whole-cell channel current (I), the holding potential (Vₕ) and the reversal potential of the channel current (Vᵣ):

$$γ = \frac{π S ( 0 ) {f}_{c}}{2 I ( {V}_{h} - {V}_{r} )}$$

### Example 2.4

### Electrical Measurements in Neurons

The 7TM domains of PsChR or CrChR2 in frame with an EYFP tag were inserted into pFUGW lentivirus vector backbone between BamHI and EcoRI sites. The lentivirus was produced by triple transfection of HEK293FT cells (Invitrogen) with the envelope plasmid pCMV-VSVG, the packaging plasmid pΔ8.9 and the pFUGW-PsChR/CrChR2-EYFP plasmids using Lipofectamine 2000 (Invitrogen), as described in Lois, et al., 2002 (Lois, C., Hong, E. J., Pease, S., Brown, E. J., and Baltimore, D. 2002. Germline transmission and tissue-specific expression of transgenes delivered by lentiviral vectors. Science 295, 868-872).

The viral titer was determined by infection of HEK cells. Hippocampi of E18 Sprague Dawley rats were obtained as part of a kit from BrainBits (Springfield, Ill.), and primary neuronal cultures were prepared using the protocol provided by the company. Cells were cultured in NbActiv4 medium (Brewer, G. J., Boehler, M. D., Jones, T. T., and Wheeler, B. C. (2008) NbActiv4 medium improvement to Neurobasal/B27 increases neuron synapse densities and network spike rates on multielectrode arrays. J. Neurosci. Methods 170, 181-187) on poly-lysine coated coverslips and supplemented with 0.4 μM all-trans retinal (final concentration, in addition to retinyl acetate present in the medium), unless otherwise indicated. Neurons were infected with the lentivirus one day after plating. Between 10 and 19 days after plating the cells were used for patch-clamp measurements with the same setup as described for HEK cells, except that neurons were bathed in Tyrode's solution (in mM): NaCl 125, KCl 2, CaCl₂ 3, MgCl₂ 1, HEPES 25, glucose 30, pH 7.3, and the pipette solution contained (in mM): KCl 135, NaCl 6, EGTA 0.35, Mg-ATP 4, HEPES 20, pH 7.25. Spiking was measured in the current clamp mode to keep the membrane voltage at approximately −65 mV.

Example 2.5

### Expression and Purification of PsChR

Expression and purification of PsChR in Pichia pastoris. The 7TM domain of PsChR (SEQ ID NO:1) with a TEV protease site at the N-terminus and a nine-His tag at the C-terminus was subcloned into the pPIC9K vector (Invitrogen, Carlsbad, Calif.) via its EcoRI and AvrII sites. P. pastoris strain SMD1168 (his4, pep4) (Invitrogen) was transformed by electroporation using the linearized resultant plasmid pPIC9K-PsChR-9His. A P. pastoris clone that grows on 4 mg/ml geneticin was selected according to the manufacturer's instructions. Protein expression and purification was carried out as described earlier for CaChR1 (described in US patent publication US20130066047; Hou, et al., 2012, ibid). Cells were grown in buffered glycerol-complex medium to OD600 2-6, transferred to buffered minimal methanol yeast medium supplemented with all-trans-retinal and induced with 0.5% methanol. After 24-30 hours, the cells were harvested by low speed centrifugation and disrupted in a bead beater (BioSpec Products, Bartlesville, Okla.). The membranes were collected by ultracentrifugation and solubilized in 1.5% (w/v) dodecyl maltoside (DDM) for 1 hour at 4° C. Non-solubilized material was removed by ultracentrifugation, and protein was purified from the supernatant using a Ni-NTA column (Qiagen, Hilden, Germany). The protein samples were concentrated in 100 mM NaCl, 0.02% DDM, 20 mM HEPES (pH 7.4) and used for measurements either directly, or after reconstitution in nanodiscs with 1,2-dimyristoyl-sn-glycero-3-phosphocholine lipid (DMPC) from Avanti Polar Lipids (Alabaster, Ala.), as described for haloarchaeal sensory rhodopsin II (Wang, J., Sasaki, J., Tsai, A. L., and Spudich, J. L. 2012. HAMP domain signal relay mechanism in a sensory rhodopsin-transducer complex. J. Biol. Chem. 287, 21316-21325).

### Example 2.6

### Absorption Spectroscopy and Flash Photolysis

Absorption spectra of partially purified PsChR in the UV-Visible range were recorded on a Cary 4000 spectrophotometer (Varian, Palo Alto, Calif.). pH titration was carried out by the addition of small volumes of 1 M NaOH, 0.5 M Tris (pH 10), 0.5 M citric acid or 1 M HCl. Light-induced absorption changes of Pichia-expressed pigment were measured with a laboratory-constructed cross-beam apparatus (Wang, W.-W., Sineshchekov, O. A., Spudich, E. N., and Spudich, J. L. (2003) Spectroscopic and photochemical characterization of a deep ocean proteorhodopsin. J. Biol. Chem. 278, 33985-33991). Excitation flashes (532 nm, 6 ns, 40 mJ) were provided by a Surelite I Nd-YAG laser (Continuum, Santa Clara, Calif.). Measuring light was from a 250-W incandescent tungsten lamp combined with a McPherson monochromator (model 272, Acton, Mass.). A Hamamatsu Photonics photomultiplier tube (model R928, Bridgewater, N.J.) was protected from excitation laser flashes by a second monochromator of the same type and additionally with 12-nm bandwidth interference filters (Oriel Instruments, Stratford, Conn.). Signals were amplified by a low noise current amplifier (model SR445A, Stanford Research Systems, Sunnyvale, Calif.) and digitized by a Digidata 1320A (Molecular Devices, Union City, Calif.) at the sampling rate 4 μs/point. The time interval between excitation flashes was 10 s, and 100 sweeps were averaged for each data point. Currents generated by PsChR in HEK293 cells show higher amplitude, smaller inactivation, and faster peak recovery than those generated by CrChR2, the molecule of choice in most optogenetic studies, whereas their kinetics is similarly fast. The higher current amplitude of PsChR is due to its higher unitary conductance, as estimated by stationary noise analysis.

### Example 2.7

### Retinal Extraction and Analysis

Utilizing a protocol adapted from previously describe methods (Sineshchekov, O. A., Trivedi, V. D., Sasaki, J., and Spudich, J. L. 2005. Photochromicity of Anabaena sensory rhodopsin, an atypical microbial receptor with a cis-retinal light-adapted form. J. Biol. Chem. 280, 14663-14668; Nack, M., Radu, I., Bamann, C., Bamberg, E., and Heberle, J. 2009. The retinal structure of channelrhodopsin-2 assessed by resonance Raman spectroscopy. FEBS Lett. 583, 3676-3680). Protein samples were kept overnight in the dark and maintained in darkness or illuminated for 2 min using a tungsten halogen light beam from an FOI-150W Illuminator (Titan Tool Supply, Buffalo, N.Y.) passed through a heat filter and a 432±5 nm interference filter. Retinal was extracted by the addition of ice-cold methanol followed by ice-cold hexane and vortexing under dim red light. Phases were separated by centrifugation and the top layer (hexane phase) was carefully withdrawn and dried under argon. The samples were dissolved in methanol and separated in 100% hexane on a Spherisorb S5 ODS2 analytical column using a Waters Delta 600 HPLC system (Waters Corporation, Milford, Mass.). Data analysis was performed with pClamp 10.2 (Molecular Devices, Union City, Calif.) and OriginPro 7 (OriginLab Corporation, Northampton, Mass.) software. PsChR demonstrated a dramatically increased level of expression and, correspondingly, the amplitude of channel currents generated in animal cells upon the addition of exogenous retinal than did CrChR. This increase was even larger than that seen with CrChR2. Therefore, the intact mammalian brain may have enough endogenous retinal to reconstitute fully functional PsChR.

### Example 2.8

### Photoreceptor Currents

Unilateral light excitation of suspensions of P. subcordiformis cells elicited characteristic photoelectric responses (FIG. 1, inset) previously detected in many freshwater flagellates. They are comprised of photoreceptor currents superimposed with the regenerative response triggered by depolarization of the plasma membrane and these photoreceptor currents are mediated by ChRs.

The action spectrum of photoreceptor currents in P. subcordiformis shows a main peak at 510 nm and a pronounced shoulder in the blue region (FIG. 1, main figure), suggesting that there are two photoreceptor pigments. The maximum of the spectral sensitivity of PsChR in HEK293 cells was at 445 nm, which strongly suggests it being one of the two receptors responsible for phototaxis in this organism.

### Example 2.9

### PsChR Primary Structure

The amino acid sequence of PsChR is comprised of 660 residues (SEQ ID NO: 3 and its N-terminal half forms the 7TM (rhodopsin) domain (SEQ ID NO: 1). Two more transmembrane helices are predicted in its C-terminal half. Its N-terminus (upstream of the 7TM domain) is relatively short and contains no predicted signal peptide. Only Cys73 (based on CrChR1 numbering (SEQ ID NO:10) is conserved out of three N-terminal Cys residues found to form disulphide bonds between protomers in the C1C2 dimer (Kato, H. E., Zhang, F., Yizhar, O., Ramakrishnan, C., Nishizawa, T., Hirata, K., Ito, J., Aita, Y., Tsukazaki, T., Hayashi, S., Hegemann, P., Maturana, A. D., Ishitani, R., Deisseroth, K., and Nureki, O. (2012) Crystal structure of the channelrhodopsin light-gated cation channel. Nature 482, 369-374). It contains all five Glu residues and a Lys residue in helix B, conserved in ChRs from C. reinhardtii and Volvox carteri (Suppl. FIG. 1). Glu and Asp residues are found, respectively, in the positions of the protonated Schiff base counterions Asp85 and Asp212 in bacteriorhodopsin (BR), and a His residue in the position of the proton donor Asp96 in BR. Glu87 of CrChR1, responsible for its pH-dependent color tuning and fast photocurrent inactivation (Tsunoda, S. P., and Hegemann, P. (2009) Glu 87 of channelrhodopsin-1 causes pH-dependent color tuning and fast photocurrent inactivation. Photochem. Photobiol. 85, 564-569), is also conserved in PsChR. The position of Tyr226/Asn187 (in CrChR1/CrChR2, respectively), identified as one of the molecular determinants of differences in spectra, desensitization, and current kinetics in response to a step-up and step-down of continuous light between CrChR1 and CrChR2 (Wang, H., Sugiyama, Y., Hikima, T., Sugano, E., Tomita, H., Takahashi, T., Ishizuka, T., and Yawo, H. 2009. Molecular determinants differentiating photocurrent properties of two channelrhodopsins from Chlamydomonas. J. Biol. Chem. 284, 5685-5696), in PsChR, is occupied by a Ser residue.

### Example 3.0

### Kinetics and Inactivation of PsChR Currents

The 7TM domain of PsChR expressed in HEK293 cells demonstrated light-gated channel activity typical of other high-efficiency ChRs (FIG. 2). The mean peak current generated by PsChR at saturating light intensity was 4.6±0.4 nA (n=27 cells), compared to 2.5±0.2 nA (n=20 cells) for CrChR2, the channelrhodopsin most frequently used in optogenetic studies (FIG. 2C). The difference between the plateau currents for these two pigments was even greater, ˜3-fold (FIG. 2C). Under continuous illumination the photocurrent decreased from a peak to a quasi-stationary level, which is also known for all other ChRs and is called light inactivation or desensitization. For PsChR the rate of this process was slower and its extent significantly less than that of CrChR2 (FIG. 2). In addition to the main phase of inactivation with the time constant (τ) ˜40 ms, which contributed 76% of the amplitude, a slow second phase with τ ˜7 s was observed (saturating light intensity, −60 mV, pH 7.4). Current inactivation after 1-s illumination, calculated as the difference between the peak and plateau current relative to the peak current, was ˜1.5-fold less for PsChR than for CrChR2 (FIG. 2D). After switching off the light, PsChR currents decayed biexponentially with τ of both phases ˜15% larger (τ₁=9.8±0.3 ms, τ₂=102±5.4 ms, n=24 cells) than those for CrChR2 currents under the same conditions (τ₁=8.3±0.6 ms, τ₂=84.9±11.3 ms, n=16 cells).

### Example 3.1

### Current Amplitude and Unitary Conductance

The amplitude of the whole-cell current measured under continuous illumination depends on the number of photoactive molecules in the cell membrane, lifetime of the open channel, and its unitary conductance. Although single channel currents generated by CrChR2 are below the limit for direct recording, their parameters could be estimated by stationary noise analysis (for methods see Feldbauer, K., Zimmermann, D., Pintschovius, V., Spitz, J., Bamann, C., and Bamberg, E. 2009. Channelrhodopsin-2 is a leaky proton pump. Proc. Natl. Acad. Sci. USA 106, 12317-12322). This approach was used to determine whether a greater plateau current of PsChR reflects an increased unitary conductance over that of CrChR2.

In PsChR-transfected cells the noise amplitude became considerably larger under continuous illumination, as compared to the dark conditions (FIG. 3A, top traces). This increase in noise was much greater than observed in CrChR2-transfected cells under the same conditions (FIG. 3A, bottom traces). The noise of control untransfected cells did not change at all after switching on the light (data not shown). The power density spectra for both light and dark conditions contained a significant 1/f component, i.e., a component inversely proportional to the frequency (FIG. 3B). However, the light-minus-dark difference could be fit with a Lorentzian function between 2 Hz and 1 kHz (FIG. 3C). The value of unitary conductance (y) for PsChR obtained from this fit was ˜3-fold larger than for CrChR2. The value for CrChR2 was close to that reported previously (Feldbauer, et al., 2009, ibid), taking into account the lower bath Na⁺ concentration in our experiments and the temperature dependence of γ. The close correlation between the whole-cell plateau current amplitudes and the calculated unitary conductances (FIG. 3D) show that the greater current amplitude of PsChR in HEK cells over that of CrChR2 is attributable to its greater unitary conductance.

### Example 3.2

### Current Peak Recovery

A general property of all characterized ChRs is that a second light pulse delivered after a short dark interval elicits a response with a smaller transient peak than that invoked by the first pulse, although the plateau level is the same for both pulses (FIG. 4A). The time course of this process is multicomponential and depends on the membrane potential and extracellular pH (Nagel, G., Szellas, T., Huhn, W., Kateriya, S., Adeishvili, N., Berthold, P., Ollig, D., Hegemann, P., and Bamberg, E. (2003) Channelrhodopsin-2, a directly light-gated cation-selective membrane channel. Proc. Natl. Acad. Sci. USA 100, 13940-13945). The recovery of the peak current generated by PsChR was faster than that of any so far tested ChR. In particular, 50% of the initial peak amplitude was recovered in a ˜30-fold shorter time than with CrChR2 measured under the same conditions (FIG. 4B).

### Example 3.3

### Ion Selectivity

Inward photocurrents generated by PsChR were almost entirely carried by Na⁺ ions, as revealed by their dramatic suppression after partial replacement of Na⁺ in the bath with non-permeable organic NMG⁺ (FIG. 5A). In comparison, only ˜80% of CrChR2 current was contributed by Na⁺, as estimated in a parallel experiment (data not shown; but see Nagel, et al., 2003, ibid; Zhang, et al., 2011, ibid). The current-voltage relationships were measured and the shifts of the reversal voltage (Vᵣ) upon a decrease of Na⁺ or H⁺ concentrations in the bath calculated for PsChR and several other ChRs for comparison. The greatest negative shifts upon Na⁺ depletion were obtained with PsChR and MvChR1 from Mesostigma viride, which indicated their highest relative permeability to Na⁺ ions over protons of all tested ChRs (FIG. 5B). However, the Na⁺ conductance of MvChR1 was inhibited by protons; peak current amplitude decreased 24% when the bath pH was changed from 7.4 to 6.4, despite a ˜7 mV positive shift of the Vᵣ, indicating that protons were also permeable through its channel. In contrast, no such inhibition was observed in PsChR: the current amplitude difference between pH 7.4 and 6.4 was <2%.

The Vᵣ of CrChR2 current shifted to a less positive value during illumination (from 15.8±1.0 mV for the initial current to 10.7±1.2 mV for the plateau current; n=6 cells). In contrast, the corresponding Vᵣ values calculated for PsChR current were approximately the same (13.1±0.6 mV and 14.1±0.6 mV, respectively; n=7 cells). PsChR demonstrated higher selectivity for Na+ ions over protons. PsChR appears to exhibit a single conductive state, in contrast to CrChR2.

### Example 3.4

### Action Spectroscopy

Action spectra of PsChR-generated currents were measured using a 50-ms light pulse of low intensity, as described earlier for MvChR1 (in US patent publication and Govorunova, et al., 2011, ibid). Its maximum was at 445 nm (FIG. 6A, black line), which makes PsChR the shortest wavelength-absorbing ChR so far characterized.

Most ChRs (except DsChR1) contain two carboxylate residues homologous to Asp85 and Asp212 in BR (Suppl. FIG. 1) that form hydrogen bonds with the Schiff base nitrogen, as revealed by the crystal structure of the C1C2 chimera and reported in (Kato, H. E., Zhang, F., Yizhar, O., Ramakrishnan, C., Nishizawa, T., Hirata, K., Ito, J., Aita, Y., Tsukazaki, T., Hayashi, S., Hegemann, P., Maturana, A. D., Ishitani, R., Deisseroth, K., and Nureki, O. 2012. Crystal structure of the channelrhodopsin light-gated cation channel. Nature 482, 369-374). The E106Q and D236N mutants were generated to neutralize each of the corresponding carboxylate positions in PsChR and their spectral sensitivities measured. Both mutations caused a red shift of the spectrum (FIG. 6A), the magnitude of which was larger upon neutralization of the Asp85 homolog (30 nm) than of the Asp212 homolog (14 nm). Similar results (red shifts of 23 and 16 nm) were found in the corresponding CrChR2 mutants, E123Q and D253N (FIG. 6B). This is in contrast to low-efficiency CaChR1, in which the corresponding mutations caused a blue spectral shift, as did threonine substitution of the Asp85 homolog in the C1V1 chimera (Yizhar, O., Fenno, L. E., Prigge, M., Schneider, F., Davidson, T. J., O'Shea, D. J., Sohal, V. S., Goshen, I., Finkelstein, J., Paz, J. T., Stehfest, K., Fudim, R., Ramakrishnan, C., Huguenard, J. R., Hegemann, P., and Deisseroth, K. (2011) Neocortical excitation/inhibition balance in information processing and social dysfunction. Nature 477, 171-178).

Acidification of the medium also caused a red shift of the PsChR action spectrum, but its magnitude was relatively smaller compared to those observed in the counterion mutants: less than 3 nm when pH was shifted from 9.0 to 7.4, and 4 nm—from 7.4 to 5.4 (FIG. 6C). Channel activity of the E106Q and D236N mutants was greatly reduced (˜44 and ˜100 fold, respectively, with respect to that of wild type).

### Example 3.5

### Characterization of Purified PsChR

PsChR was expressed in P. pastoris and partially purified in detergent with yields of 0.2-0.3 mg/l of yeast culture. The absorption spectrum of purified PsChR in DDM showed an absorption maximum at 437 nm and ˜90 nm half bandwidth (FIG. 7A, solid line). The absorption spectrum shifted less than 1-2 nm to longer wavelengths when pigment molecules were incorporated into lipid nanodiscs (FIG. 7A, dashed line). Both spectra lacked obvious vibrational fine structure, characteristic of the other relatively blue-shifted microbial rhodopsins CrChR2 (Bamann, C., Kirsch, T., Nagel, G., and Bamberg, E. 2008. Spectral characteristics of the photocycle of channelrhodopsin-2 and its implication for channel function. J. Mol. Biol. 375, 686-694; Ritter, E., Stehfest, K., Berndt, A., Hegemann, P., and Bartl, F. J. 2008. Monitoring light-induced structural changes of Channelrhodopsin-2 by UV-visible and Fourier transform infrared spectroscopy. J. Biol. Chem. 283, 35033-35041) and haloarchaeal sensory rhodopsin II (Takahashi, T., Yan, B., Mazur, P., Derguini, F., Nakanishi, K., and Spudich, J. L. (1990) Color regulation in the archaebacterial phototaxis receptor phoborhodopsin (sensory rhodopsin II). Biochemistry 29, 8467-8474).

The absorption spectrum of purified PsChR also shifted to longer wavelengths upon acidification of the medium, as did the action spectrum of channel currents. Interestingly, the maximum at 445 nm (the peak of the action spectrum) was observed at the non-physiological pH ˜4.3 (FIG. 7B). Titration of the peak revealed at least 3 titratable groups. The shape of the differential signal for the most alkaline transition (data not shown) indicated that it reflects deprotonation of the Schiff base, hence fitting of the titration data was performed with the base level parameter fixed at 360 nm. The pKₐ of this transition was ˜10.5, and those of the other two transitions were 3.8 and 6.6. Neutralization of Glu106 caused a larger red shift of the action spectra of the two mutants tested (FIG. 6A, red line). The transition with pKₐ ˜6.6 and smaller amplitude may correspond to protonation of Asp236, a neutral mutation of which caused a smaller red spectral shift (FIG. 6A, green line).

Retinal extraction experiments showed that the ratio of all-trans to 13-cis isomer in dark-adapted PsChR was ˜75:25. No significant changes in the isomer composition were observed upon light adaptation. We carried out spectroscopic measurements to resolve possible small-amplitude changes in PsChR that fall below the detection limit of retinal extraction and to follow their time course. Dark adaptation of PsChR led to a blue spectral shift with a typical light-dark adaptation shape of the difference spectrum (FIG. 7C, inset). The amount of 13-cis retinal increased in the dark. However, its magnitude in PsChR was dramatically smaller as judged from the difference spectrum (only 3% of the total absorbance). The time constant of dark adaptation in PsChR was ˜26 min (FIG. 7C, main figure).

FIG. 8A shows the spectra of laser flash-induced absorbance changes in detergent-purified PsChR. Although no positive signal was observed in the short-wavelength region at room temperature, an increase in the absorption at 510 nm at the expense of a decrease at 380 nm at the early stages of the photocycle suggests contribution of an early M-like state, the absorption of which strongly overlaps with the absorption of the unphotolysed state. Therefore, kinetics of this intermediate was followed at 5° C. to improve the time resolution (FIG. 8B). Even at this low temperature the rates of the M intermediate formation and decay were faster than those reported in CrChR2 (Verhoefen, et al., 2010, ibid). Consequently, maximal accumulation of the M intermediate of PsChR was observed at 50 μs, as compared to 200 μs in CrChR2. The main photoproduct (N/O-like) in the PsChR photocycle is red-shifted and biphasic in both its rise and decay. The rise and the fast component of the decay roughly correlated with the opening and closing of the channel measured in HEK cells (FIG. 8C).

### Example 3.6

### Function of PsChR in Neurons

To test whether PsChR is relevant for optogenetic applications, we examined its performance in cultured hippocampal neurons. Neurons expressing PsChR were capable of firing action potentials upon light stimulation with brief light pulses at frequencies up to 50 Hz (FIG. 9A), which is the upper limit for pyramidal neurons (Zhang, F., Prigge, M., Beyriere, F., Tsunoda, S. P., Mattis, J., Yizhar, O., Hegemann, P., and Deisseroth, K. (2008) Red-shifted optogenetic excitation: a tool for fast neural control derived from Volvox carteri. Nat. Neurosci. 11, 631-633). Expression of both PsChR and CrChR2, as judged by the tag fluorescence, was increased when the cultures were supplemented with all-trans retinal (0.4 μM final concentration) in addition to 0.5 μM retinyl acetate present in the culture medium. Photoinduced channel currents generated in neurons by PsChR were increased ˜9-fold, and those by CrChR2—by ˜5-fold in cultures with additional retinal, which was similar to results obtained in HEK293 cells (˜12- and ˜2.4-fold increase for PsChR and CrChR2, respectively).

Upon 1-Hz excitation the rate of the membrane depolarization decreased significantly more slowly and to a lesser degree for PsChR than for CrChR2 (FIG. 9B and C, left axes). Correspondingly, the probability of spike generation driven by this depolarization only slightly decreased during a 20-s period of excitation in PsChR-expressing neurons, whereas in CrChR2-expressing neurons it rapidly dropped to a much lower level (FIGS. 9B and C, bars, right axes). Thus it was shown that PsChR can be expressed in cultured hippocampal neurons and can be used to drive their spiking activity by light excitation. Moreover, a faster peak recovery of PsChR-generated currents provides an advantage in certain optogenetic applications.

### REFERENCES

The following literature citations as well as those cited above are incorporated by reference to the extent that they support the present disclosure.

### Biophys

1. Spudich, J. L., and K.-H. Jung. 2005. Microbial rhodopsins: phylogenetic and functional diversity. In Handbook of Photosensory Receptors. Wiley-VCH, Weinheim, Germany. 1-23.

2. Sineshchekov, O. A., Jung, K.-H., and Spudich, J. L. (2002) Two rhodopsins mediate phototaxis to low- and high-intensity light in Chlamydomonas reinhardtii. Proc. Natl. Acad. Sci. U.S.A. 99, 8689-8694.

3. Govorunova, E. G., K. H. Jung, J. L. Spudich. 2004. Chlamydomonas sensory rhodopsins A and B: cellular content and role in photo-phobic responses. Biophys. J. 86:2342-2349.

4. Berthold, P., S. P. Tsunoda, P. Hegemann. 2008. Channelrhodopsin-1 initiates phototaxis and photophobic responses in chlamydomonas by immediate light-induced depolarization. Plant Cell. 20:1665-1677.

5. Litvin, F. F., O. A. Sineshchekov, and V. A. Sineshchekov. 1978. Photo-receptor electric potential in the phototaxis of the alga Haematococcus pluvialis. Nature. 271:476-478.

6. Nagel, G., D. Ollig, P. Hegemann. 2002. Channelrhodopsin-1: a light-gated proton channel in green algae. Science. 296:2395-2398.

7. Nagel, G., T. Szellas, E. Bamberg. 2003. Channelrhodopsin-2, a directly light-gated cation-selective membrane channel. Proc. Natl. Acad. Sci. USA. 100:13940-13945.

8. Chow, B. Y., A. S. Chuong, E. S. Boyden. 2011. Synthetic physiology strategies for adapting tools from nature for genetically targeted control of fast biological processes. Methods Enzymol. 497:425-443.

9. Yizhar, O., L. Fenno, F. Zhang, P. Hegemann, and K. Diesseroth. 2011. Microbial opsins: a family of single-component tools for optical control of neural activity. Cold Spring Harb. Protoc. 2011: top102.

10. Zhang, F., J. Vierock, K. Deisseroth. 2011. The microbial opsin family of optogenetic tools. Cell. 147:1446-1457.

11. Mogi, T., L. J. Stern, H. G. Khorana. 1988. Aspartic acid substitutions affect proton translocation by bacteriorhodopsin. Proc. Natl. Acad. Sci. USA. 85:4148-4152.

12. Butt, H. J., K. Fendler, D. Oesterhelt. 1989. Aspartic acids 96 and 85 play a central role in the function of bacteriorhodopsin as a proton pump. EMBO J. 8:1657-1663.

13. Subramaniam, S., T. Marti, and H. G. Khorana. 1990. Protonation state of Asp (Glu)-85 regulates the purple-to-blue transition in bacteriorhodopsin mutants Arg-82/Ala and Asp-85/Glu: the blue form is inactive in proton translocation. Proc. Natl. Acad. Sci. USA. 87:1013-1017.

14. Spudich, E. N., W. Zhang, J. L. Spudich. 1997. Constitutive signaling by the phototaxis receptor sensory rhodopsin II from disruption of its protonated Schiff base-Asp-73 interhelical salt bridge. Proc. Natl. Acad. Sci. USA. 94:4960-4965.

15. Sasaki, J., T. Nara, J. L. Spudich. 2007. Constitutive activity in chimeras and deletions localize sensory rhodopsin II/HtrII signal relay to the membrane-inserted domain. Mol. Microbiol. 66:1321-1330.

16. Holterhues, J., E. Bordignon, H. J. Steinhoff. 2011. The signal transfer from the receptor NpSRII to the transducer NpHtrII is not hampered by the D75N mutation. Biophys. J. 100:2275-2282.

17. Sineshchekov, O. A., J. Sasaki, J. L. Spudich. 2008. A Schiff base connectivity switch in sensory rhodopsin signaling. Proc. Natl. Acad. Sci. USA. 105:16159-16164.

18. Sineshchekov, O. A., J. Sasaki, J. L. Spudich. 2010. Attractant and repellent signaling conformers of sensory rhodopsin-transducer complexes. Biochemistry. 49:6696-6704.

19. Sasaki, J., A. L. Tsai, and J. L. Spudich. 2011. Opposite displacement of helix F in attractant and repellent signaling by sensory rhodopsin-Htr complexes. J. Biol. Chem. 286:18868-18877.

20. Sharma, A. K., J. L. Spudich, and W. F. Doolittle. 2006. Microbial rhodopsins: functional versatility and genetic mobility. Trends Microbiol. 14:463-469.

21. Kato, H. E., F. Zhang, O. Nureki. 2012. Crystal structure of the channelrhodopsin light-gated cation channel. Nature. 482:369-374.

22. Luecke, H., B. Schobert, J. L. Spudich. 2001. Crystal structure of sensory rhodopsin II at 2.4 A°: insights into color tuning and transducer interaction. Science. 293:1499-1503.

23. Feldbauer, K., D. Zimmermann, E. Bamberg. 2009. Channelrhodopsin-2 is a leaky proton pump. Proc. Natl. Acad. Sci. USA. 106: 12317-12322.

24. Nack, M., Radu, I., Bamann, C., Bamberg, E. ?, and Heberle, J. (2012) Kinetics of proton release and uptake by channelrhodopsin-2. FEBS Lett. 586:1344-1348.

25. Trissl, H.-W. 1990. Photoelectric measurements of purple membranes. Photochem. Photobiol. 51:793-818.

26. Kaulen, A. D. 2000. Electrogenic processes and protein conformational changes accompanying the bacteriorhodopsin photocycle. Biochim. Biophys. Acta. 1460:204-219.

27. De'r, A., and L. Keszthelyi. 2001. Charge motion during the photocycle of bacteriorhodopsin. Biochemistry (Mosc.). 66:1234-1248.

28. Drachev, L. A., A. D. Kaulen, and V. P. Skulachev. 1978. Time resolution of the intermediate steps in the bacteriorhodopsin-linked electrogenesis. FEBS Lett. 87:161-167.

29. Fahr, A., P. Lauger, and E. Bamberg. 1981. Photocurrent kinetics of purple-membrane sheets bound to planar bilayer membranes. J. Membr. Biol. 60:51-62.

30. Keszthelyi, L., and P. Ormos. 1980. Electric signals associated with the photocycle of bacteriorhodopsin. FEBS Lett. 109:189-193.

31. Friedrich, T., S. Geibel, E. Bamberg. 2002. Proteorhodopsin is a light-driven proton pump with variable vectoriality. J. Mol. Biol. 321:821-838.

32. Va'ro', G., L. S. Brown, J. K. Lanyi. 2003. Characterization of the photochemical reaction cycle of proteorhodopsin. Biophys. J. 84: 1202-1207.

33. Sineshchekov, O. A., and J. L. Spudich. 2004. Light-induced intramolecular charge movements in microbial rhodopsins in intact E. coli cells. Photochem. Photobiol. Sci. 3:548-554.

34. Nagel, G., B. Mo\ddot{}ckel, E. Bamberg. 1995. Functional expression of bacteriorhodopsin in oocytes allows direct measurement of voltage dependence of light induced H [symbol] pumping. FEBS Lett. 377:263-266.

35. McLachlan, J. 1960. The culture of Dunaliella tertiolecta Butcher: a euryhaline organism. Can. J. Microbiol. 6:367-379.

36. Haghjou, M. M., M. Shariati, and N. Smirnoff. 2009. The effect of acute high light and low temperature stresses on the ascorbate-gluta-thione cycle and superoxide dismutase activity in two Dunaliella salina strains. Physiol. Plant. 135:272-280.

37. Optogenetics Resource Center. http://www.stanford.edu/group/dlab/optogenetics/ (Karl Deisseroth, Stanford University, CA, USA).

38. Hou, S. Y., E. G. Govorunova, J. L. Spudich. 2012. Diversity of Chlamydomonas channelrhodopsins. Photochem. Photobiol. 88: 119-128.

39. Wang, W.-W., O. A. Sineshchekov, J. L. Spudich. 2003. Spectro-scopic and photochemical characterization of a deep ocean proteorhodopsin. J. Biol. Chem. 278:33985-33991.

40. Chow, B. Y., X. Han, E. S. Boyden. 2010. High-performance genetically targetable optical neural silencing by light-driven proton pumps. Nature. 463:98-102.

41. Husson, S. J., J. F. Liewald, A. Gottschalk. 2012. Microbial light-activatable proton pumps as neuronal inhibitors to functionally dissect neuronal networks in C. elegans. PLoS ONE. 7:e40937.

42. Verhoefen, M. K., C. Bamann, J. Wachtveitl. 2010. The photocycle of channelrhodopsin-2: ultrafast reaction dynamics and subsequent reaction steps. ChemPhysChem. 11:3113-3122.

43. Otto, H., T. Marti, M. P. Heyn. 1990. Substitution of amino acids Asp-85, Asp-212, and Arg-82 in bacteriorhodopsin affects the proton release phase of the pump and the pK of the Schiff base. Proc. Natl. Acad. Sci. USA. 87:1018-1022.

44. Gergely, C., and G. Varo. 1992. Charge motions in the D85N and D212N mutants of bacteriorhodopsin. In Structures and Functions of Retinal Proteins. J. L. Rigaud, editor. John Libbey, Paris. 193-196.

45. Balashov, S. P., R. Govindjee, Y. Feng. 1993. Effect of the arginine-82 to alanine mutation in bacteriorhodopsin on dark adaptation, proton release, and the photochemical cycle. Biochemistry. 32: 10331-10343.

46. Govindjee, R., S. Misra, D. R. Menick. 1996. Arginine-82 regulates the pKa of the group responsible for the light-driven proton release in bacteriorhodopsin. Biophys. J. 71:1011-1023.

47. Ernst, O. P., P. A. Sa'nchez Murcia, P. Hegemann. 2008. Photoactivation of channelrhodopsin. J. Biol. Chem. 283:1637-1643.

48. Kianianmomeni, A., K. Stehfest, A. Hallmann. 2009. Channelrhodopsins of Volvox carteri are photochromic proteins that are specifically expressed in somatic cells under control of light, temperature, and the sex inducer. Plant Physiol. 151:347-366.

49. Berndt, A., M. Prigge, P. Hegemann. 2010. Two open states with progressive proton selectivities in the branched channelrhodopsin-2 photocycle. Biophys. J. 98:753-761.

50. Zhang, F., L. P. Wang, K. Deisseroth. 2006. Channelrhodopsin-2 and optical control of excitable cells. Nat. Methods. 3:785-792.

51. Bamann, C., T. Kirsch, E. Bamberg. 2008. Spectral characteristics of the photocycle of channelrhodopsin-2 and its implication for channel function. J. Mol. Biol. 375:686-694.

52. Govorunova, E. G., E. N. Spudich, J. L. Spudich. 2011. New channelrhodopsin with a red-shifted spectrum and rapid kinetics from Mesostigma viride. MBio. 2: e00115-11.

53. Eisenhauer, K., J. Kuhne, K. Gerwert. 2012. In channelrhodopsin-2 Glu-90 is crucial for ion selectivity and is deprotonated during the photocycle. J. Biol. Chem. 287:6904-6911.

54. Lin, J. Y., M. Z. Lin, P. Steinbach, and R. Y. Tsien. 2009. Characterization of engineered channelrhodopsin variants with improved properties and kinetics. Biophys. J. 96:1803-1814.

55. Sineshchekov, O. A., E. G. Govorunova, and J. L. Spudich. 2009. Photosensory functions of channelrhodopsins in native algal cells. Photochem. Photobiol. 85:556-563.

### JBC:

1. Sineshchekov, O. A., and Spudich, J. L. (2005) Sensory rhodopsin signaling in green flagellate algae, in Handbook of Photosensory Receptors, pp. 25-42, Wiley-VCH, Weinheim

2. Hegemann, P. (2008) Algal sensory photoreceptors. Annu. Rev. Plant. Biol. 59, 167-189

3. Litvin, F. F., Sineshchekov, O. A., and Sineshchekov, V. A. (1978) Photoreceptor electric potential in the phototaxis of the alga Haematococcus pluvialis. Nature 271, 476-478

4. Sineshchekov, O. A., Jung, K.-H., and Spudich, J. L. (2002) Two rhodopsins mediate phototaxis to low- and high-intensity light in Chlamydomonas reinhardtii. Proc. Natl. Acad. Sci. U.S.A. 99, 8689-8694.

5. Govorunova, E. G., Jung, K.-W., Sineshchekov, O. A., and Spudich, J. L. (2004) Chlamydomonas sensory rhodopsins A and B. Cellular content and role in photophobic responses. Biophys. J. 86, 2342-2349.

6. Sineshchekov, O. A., Govorunova, E. G., Der, A., Keszthelyi, L., and Nultsch, W. (1992) Photoelectric responses in phototactic flagellated algae measured n cell suspension. J. Photochem. Photobiol. B Biol. 13, 119-134.

7. Nagel, G., Ollig, D., Fuhrmann, M., Kateriya, S., Musti, A. M., Bamberg, E., and Hegemann, P. (2002) Channelrhodopsin-1. A light-gated proton channel in green algae. Science 296, 2395-2398.

8. Nagel, G., Szellas, T., Huhn, W., Kateriya, S., Adeishvili, N., Berthold, P., Ollig, D., Hegemann, P., and Bamberg, E. (2003) Channelrhodopsin-2, a directly light-gated cation-selective membrane channel. Proc. Natl. Acad. Sci. U.S.A. 100, 13940-13945.

9. Deisseroth, K. (2011) Optogenetics. Nat. Methods 8, 26-29.

10. Mei, Y., and Zhang, F. (2012) Molecular tools and approaches for optogenetics. Biol. Psychiatry 71, 1033-1038.

11 Hegemann, P., and Nagel, G. (2013) From channelrhodopsins to optogenetics. EMBO Mol. Med. 5, 171-176.

12. Yawo, H., Asano, T., Sakai, S., and Ishizuka, T. (2013) Optogenetic manipulation of neural and non-neural functions. Dev. Growth Differ. 55, 474-490.

13. Hegemann, P., and Moglich, A. (2011) Channelrhodopsin engineering and exploration of new optogenetic tools. Nat. Methods 8, 39-42.

14. Mattis, J., Tye, K. M., Ferenczi, E. A., Ramakrishnan, C., O'Shea, D. J., Prakash, R., Gunaydin, L. A., Hyun, M., Fenno, L. E., Gradinaru, V., Yizhar, O., and Deisseroth, K. (2012) Principles for applying optogenetic tools derived from direct comparative analysis of microbial opsins. Nat. Methods 9, 159-172.

15. Lin, J. Y., Lin, M. Z., Steinbach, P., and Tsien, R. Y. (2009) Characterization of engineered channelrhodopsin variants with improved properties and kinetics. Biophys. J. 96, 1803-1814.

16. Berndt, A., Schoenenberger, P., Mattis, J., Tye, K. M., Deisseroth, K., Hegemann, P., and Oertner, T. G. (2011) High-efficiency channelrhodopsins for fast neuronal stimulation at low light levels. Proc. Natl. Acad. Sci. U.S.A. 108, 7595-7600.

17. Yizhar, O., Fenno, L. E., Prigge, M., Schneider, F., Davidson, T. J., O'Shea, D. J., Sohal, V. S., Goshen, I., Finkelstein, J., Paz, J. T., Stehfest, K., Fudim, R., Ramakrishnan, C., Huguenard, J. R., Hegemann, P., and Deisseroth, K. (2011) Neocortical excitation/inhibition balance in information processing and social dysfunction. Nature 477, 171-178.

18. Prigge, M., Schneider, F., Tsunoda, S. P., Shilyansky, C., Wietek, J., Deisseroth, K., and Hegemann, P. (2012) Color-tuned channelrhodopsins for multiwavelength optogenetics. J. Biol. Chem. 287, 31804-31812.

19. Sineshchekov, O. A., Govorunova, E. G., Wang, J., Li, H., and Spudich, J. L. (2013) Intramolecular proton transfer in channelrhodopsins. Biophys. J. 104, 807-817.

20. Zhang, F., Prigge, M., Beyrie're, F., Tsunoda, S. P., Mattis, J., Yizhar, O., Hegemann, P., and Deisseroth, K. (2008) Red-shifted optogenetic excitation. A tool for fast neural control derived from Volvox carteri. Nat. Neurosci. 11, 631-633.

21. Han, X., Qian, X., Stern, P., Chuong, A. S., and Boyden, E. S. (2009) Informational lesions. Optical perturbation of spike timing and neural synchrony via microbial opsin gene fusions. Front. Mol. Neurosci. 2, 12.

22. Tang, W., Ehrlich, I., Wolff, S. B., Michalski, A. M., Wolfl, S., Hasan, M. T., Luthi, A., and Sprengel, R. (2009) Faithful expression of multiple proteins via 2A-peptide self-processing. A versatile and reliable method for manipulating brain circuits. J. Neurosci. 29, 8621-8629.

23. Kleinlogel, S., Terpitz, U., Legrum, B., Go\ddot{}kbuget, D., Boyden, E. S., Bamann, C., Wood, P. G., and Bamberg, E. (2011) A gene-fusion strategy for stoichiometric and co-localized expression of light-gated membrane proteins. Nat. Methods 8, 1083-1088.

24. Tsuda, S., Kee, M. Z., Cunha, C., Kim, J., Yan, P., Loew, L. M., and Augustine, G. J. (2013) Probing the function of neuronal populations. Combining micromirror-based optogenetic photostimulation with voltage-sensitive dye imaging. Neurosci. Res. 75, 76-81.

25. Li, Y., and Tsien, R. W. (2012) pHTomato, a red, genetically encoded indicator that enables multiplex interrogation of synaptic activity. Nat. Neurosci. 15, 1047-1053.

26. Collot, M., Loukou, C., Yakovlev, A. V., Wilms, C. D., Li, D., Evrard, A., Zamaleeva, A., Bourdieu, L., Le'ger, J. F., Ropert, N., Eilers, J., Oheim, M., Feltz, A., and Mallet, J. M. (2012) Calcium rubies. A family of red-emitting functionalizable indicators for two-photon Ca2+ imaging. J. Am. Chem. Soc. 134, 14923-14931.

27. Wu, J., Liu, L., Matsuda, T., Zhao, Y., Rebane, A., Drobizhev, M., Chang, Y. F., Araki, S., Arai, Y., March, K., Hughes, T. E., Sagou, K., Miyata, T., Nagai, T., Li, W. H., and Campbell, R. E. (2013) Improved orange and red Ca2+ indicators and photophysical considerations for optogenetic applications. ACS Chem. Neurosci. 4, 963-972.

28. McLachlan, J. (1960) The culture of Dunaliella tertiolecta Butcher. A euryhaline organism. Can. J. Microbiol. 6, 367-379.

29. Feldbauer, K., Zimmermann, D., Pintschovius, V., Spitz, J., Bamann, C., and Bamberg, E. (2009) Channelrhodopsin-2 is a leaky proton pump. Proc. Natl. Acad. Sci. U.S.A. 106, 12317-12322.

30. Cherny, V. V., Murphy, R., Sokolov, V., Levis, R. A., and DeCoursey, T. E. (2003) Properties of single voltage-gated proton channels in human eosinophils estimated by noise analysis and by direct measurement. J. Gen. Physiol. 121, 615-628.

31. Gray, P. (1994) Analysis of whole cell currents to estimate the kinetics and amplitude of underlying unitary events. Relaxation and “noise” analysis, in Microelectrode Techniques: The Plymouth Workshop Handbook (Ogden, D. C., ed) pp. 189-207, Company of Biologists, Cambridge, UK.

32. Lois, C., Hong, E. J., Pease, S., Brown, E. J., and Baltimore, D. (2002) Germ-line transmission and tissue-specific expression of transgenes delivered by lentiviral vectors. Science 295, 868-872.

33. Hou, S. Y., Govorunova, E. G., Ntefidou, M., Lane, C. E., Spudich, E. N., Sineshchekov, O. A., and Spudich, J. L. (2012) Diversity of Chlamydomonas channelrhodopsins. Photochem. Photobiol. 88, 119-128.

34. Wang, J., Sasaki, J., Tsai, A. L., and Spudich, J. L. (2012) HAMP domain signal relay mechanism in a sensory rhodopsin-transducer complex. J. Biol. Chem. 287, 21316-21325.

35. Wang, W.-W., Sineshchekov, O. A., Spudich, E. N., and Spudich, J. L. (2003) Spectroscopic and photochemical characterization of a deep ocean proteorhodopsin. J. Biol. Chem. 278, 33985-33991.

36. Sineshchekov, O. A., Trivedi, V. D., Sasaki, J., and Spudich, J. L. (2005) Photochromicity of Anabaena sensory rhodopsin, an atypical microbial receptor with a cis-retinal light-adapted form. J. Biol. Chem. 280, 14663-14668.

37. Nack, M., Radu, I., Bamann, C., Bamberg, E., and Heberle, J. (2009) The retinal structure of channelrhodopsin-2 assessed by resonance Raman spectroscopy. FEBS Lett. 583, 3676-3680.

38. Sineshchekov, O. A., Govorunova, E. G., Jung, K.-H., Zauner, S., Maier, U.-G., and Spudich, J. L. (2005) Rhodopsin-mediated photoreception in cryptophyte flagellates. Biophys. J. 89, 4310-4319.

39. Kreimer, G. (1994) Cell biology of phototaxis in flagellate algae. Int. Rev. Cytol. 148, 229-310.

40. Govorunova, E. G., Spudich, E. N., Lane, C. E., Sineshchekov, O. A., and Spudich, J. L. (2011) New channelrhodopsin with a red-shifted spectrum and rapid kinetics from Mesostigma viride. mBio 2, e00115-00111.

41. Berthold, P., Tsunoda, S. P., Ernst, O. P., Mages, W., Gradmann, D., and Hegemann, P. (2008) Channelrhodopsin-1 initiates phototaxis and photophobic responses in Chlamydomonas by immediate light-induced depolarization. Plant Cell 20, 1665-1677.

42. Halldal, P. (1958) Action spectra of phototaxis and related problems in Volvocales, Ulva gametes and Dinophyceae. Physiol. Plantarum 11, 118-153.

43. Kato, H. E., Zhang, F., Yizhar, O., Ramakrishnan, C., Nishizawa, T., Hirata, K., Ito, J., Aita, Y., Tsukazaki, T., Hayashi, S., Hegemann, P., Maturana, A. D., Ishitani, R., Deisseroth, K., and Nureki, O. (2012) Crystal structure of the channelrhodopsin light-gated cation channel. Nature 482, 369-374.

44. Zhang, F., Vierock, J., Yizhar, O., Fenno, L. E., Tsunoda, S., Kianianmomeni, A., Prigge, M., Berndt, A., Cushman, J., Polle, J., Magnuson, J., Hegemann, P., and Deisseroth, K. (2011) The microbial opsin family of optogenetic tools. Cell 147, 1446-1457.

45. Tsunoda, S. P., and Hegemann, P. (2009) Glu 87 of channelrhodopsin-1 causes pH-dependent color tuning and fast photocurrent inactivation. Photochem. Photobiol. 85, 564-569.

46. Wang, H., Sugiyama, Y., Hikima, T., Sugano, E., Tomita, H., Takahashi, T., Ishizuka, T., and Yawo, H. (2009) Molecular determinants differentiating photocurrent properties of two channelrhodopsins from Chlamydomonas. J. Biol. Chem. 284, 5685-5696.

47. Sauve', R., and Szabo, G. (1985) Interpretation of 1/f fluctuations in ion conducting membranes. J. Theor. Biol. 113, 501-516.

48. Berndt, A., Prigge, M., Gradmann, D., and Hegemann, P. (2010) Two open states with progressive proton selectivities in the branched channelrhodopsin-2 photocycle. Biophys. J. 98, 753-761.

49. Honig, B., Greenberg, A. D., Dinur, U., and Ebrey, T. G. (1976) Visual-pigment spectra. Implications of the protonation of the retinal Schiff base. Biochemistry 15, 4593-4599.

50. Subramaniam, S., Marti, T., and Khorana, H. G. (1990) Protonation state of Asp (Glu)-85 regulates the purple-to-blue transition in bacteriorhodopsin mutants Arg-82 3 Ala and Asp-85 3 Glu. The blue form is inactive in proton translocation. Proc. Natl. Acad. Sci. U.S.A. 87, 1013-1017.

51. Marti, T., Ro\ddot{}sselet, S. J., Otto, H., Heyn, M. P., and Khorana, H. G. (1991) The retinylidene Schiff base counterion in bacteriorhodopsin. J. Biol. Chem. 266, 18674-18683.

52. Gergely, C., Ganea, C., Sza'raz, S., and Va'ro', G. (1995) Charge motions studied in the bacteriorhodopsin mutants D85N and D212N. J. Photochem. Photobiol. B Biol. 27, 27-32.

53. Gunaydin, L. A., Yizhar, O., Berndt, A., Sohal, V. S., Deisseroth, K., and Hegemann, P. (2010) Ultrafast optogenetic control. Nat. Neurosci. 13, 387-392.

54. Bamann, C., Kirsch, T., Nagel, G., and Bamberg, E. (2008) Spectral characteristics of the photocycle of channelrhodopsin-2 and its implication for channel function. J. Mol. Biol. 375, 686-694.

55. Ritter, E., Stehfest, K., Berndt, A., Hegemann, P., and Bartl, F. J. (2008) Monitoring light-induced structural changes of Channelrhodopsin-2 by UV-visible and Fourier transform infrared spectroscopy. J. Biol. Chem. 283, 35033-35041.

56. Takahashi, T., Yan, B., Mazur, P., Derguini, F., Nakanishi, K., and Spudich, J. L. (1990) Color regulation in the archaebacterial phototaxis receptor phoborhodopsin (sensory rhodopsin II). Biochemistry 29, 8467-8474.

57. Pettei, M. J., Yudd, A. P., Nakanishi, K., Henselman, R., and Stoeckenius, W. (1977) Identification of retinal isomers isolated from bacteriorhodopsin. Biochemistry 16, 1955-1959.

58. Ohno, K., Takeuchi, Y., and Yoshida, M. (1977) Effect of light-adaptation on the photoreaction of bacteriorhodopsin from Halobacterium halobium. Biochim. Biophys. Acta 462, 575-582.

59. Verhoefen, M. K., Bamann, C., Blo\ddot{}cher, R., Fo\ddot{}rster, U., Bamberg, E., and Wachtveitl, J. (2010) The photocycle of channelrhodopsin-2. Ultrafast reaction dynamics and subsequent reaction steps. Chemphyschem 11, 3113-3122.

60. Suzuki, T., Yamasaki, K., Fujita, S., Oda, K., Iseki, M., Yoshida, K., Watanabe, M., Daiyasu, H., Toh, H., Asamizu, E., Tabata, S., Miura, K., Fukuzawa, H., Nakamura, S., and Takahashi, T. (2003) Archaeal-type rhodopsins in Chlamydomonas. Model structure and intracellular localization. Biochem. Biophys. Res. Commun. 301, 711-717.

61. Kianianmomeni, A., Stehfest, K., Nematollahi, G., Hegemann, P., and Hallmann, A. (2009) Channelrhodopsins of Volvox carteri are photochromic proteins that are specifically expressed in somatic cells under control of light, temperature, and the sex inducer. Plant Physiol. 151, 347-366.

62. Spudich, J. L., Yang, C.-S., Jung, K.-H., and Spudich, E. N. (2000) Retinylidene proteins. Structures and functions from archaea to humans. Annu. Rev. Cell Dev. Biol. 16, 365-392.

63. Gradmann, D., Berndt, A., Schneider, F., and Hegemann, P. (2011) Rectification of the channelrhodopsin early conductance. Biophys. J. 101, 1057-1068.

64. Ruffert, K., Himmel, B., Lall, D., Bamann, C., Bamberg, E., Betz, H., and Eulenburg, V. (2011) Glutamate residue 90 in the predicted transmembrane domain 2 is crucial for cation flux through channelrhodopsin 2. Biochem. Biophys. Res. Commun. 410, 737-743.

65. Plazzo, A. P., De Franceschi, N., Da Broi, F., Zonta, F., Sanasi, M. F., Filippini, F., and Mongillo, M. (2012) Bioinformatic and mutational analysis of channelrhodopsin-2 cation conducting pathway. J. iol. Chem. 287, 4818-4825.

66. Richards, R., and Dempski, R. E. (2012) Re-introduction of transmembrane serine residues reduce the minimum pore diameter of channelrhodopsin-2. PLoS One 7, e50018.

67. Ullrich, S., Gueta, R., and Nagel, G. (2013) Degradation of channelopsin-2 in the absence of retinal and degradation resistance in certain mutants. Biol. Chem. 394, 271-280.

68. Nagel, G., Brauner, M., Liewald, J. F., Adeishvili, N., Bamberg, E., and Gottschalk, A. (2005) Light activation of channelrhodopsin-2 in excitable cells of Caenorhabditis elegans triggers rapid behavioral responses. Curr. Biol. 15, 2279-2284.

69. Schroll, C., Riemensperger, T., Bucher, D., Ehmer, J., Voller, T., Erbguth, K., Gerber, B., Hendel, T., Nagel, G., Buchner, E., and Fiala, A. (2006) Light-induced activation of distinct modulatory neurons triggers appetitive or aversive learning in Drosophila larvae. Curr. Biol. 16, 1741-1747.

70. Brewer, G. J., Boehler, M. D., Jones, T. T., and Wheeler, B. C. (2008) NbActiv4 medium improvement to Neurobasal/B27 increases neuron synapse densities and network spike rates on multielectrode arrays. J. Neurosci. Methods 170, 181-187.

Without further elaboration, it is believed that one skilled in the art can, using the description herein, utilize the present methods to its fullest extent. The embodiments described herein are to be construed as illustrative and not as constraining the remainder of the disclosure in any way whatsoever. While preferred embodiments have been shown and described, many variations and modifications thereof can be made by one skilled in the art without departing from the spirit and teachings of the presently disclosed methods. Accordingly, the scope of protection is not limited by the description set out above, but is only limited by the claims, including all equivalents of the subject matter of the claims. The disclosures of all patents, patent applications and publications cited herein are hereby incorporated herein by reference, to the extent that they are consistent with the present disclosure set forth herein.

What is claimed is:

1. A light gated channel comprising a peptide sequence with at least 95% homology to SEQ ID NO: 1 or SEQ ID NO: 3.

2. The light gated channel of claim 1, wherein the peptide sequence is that of SEQ ID NO: 1 or SEQ ID NO: 3.

3. A vector comprising a polynucleotide sequence that encodes a peptide sequence operatively linked to a heterologous promoter sequence.

4. The vector of claim 3, wherein the peptide sequence is SEQ ID NO: 1 or SEQ ID NO: 3.

5. A recombinant host cell comprising the vector of claim 3.

6. The recombinant host cell of claim 4, wherein said host cell is an isolated human cell.

7. The recombinant host cell of claim 4, wherein said host cell is a non-human mammalian cell.

8. The recombinant host cell of claim 4, wherein said host cell is a bacterial cell.

9. The recombinant host cell of claim 4, wherein said host cell is a yeast cell.

10. The recombinant host cell of claim 4, wherein said host cell is an insect cell.

11. The recombinant host cell of claim 4, wherein said host cell is a plant cell.

12. A method of restoring photosensitivity to a retina of a subject suffering from vision loss or blindness, said method comprising:
(a) delivering to the retina of said subject the vector of claim 3, wherein said vector encodes a rhodopsin domain of a channelrhodopsin expressible in a retinal neuron; and
(b) expressing said vector in said retinal neuron, wherein the expressed rhodopsin renders said retinal neuron photosensitive, and restores photosensitivity to said retina or a portion thereof.

13. The method of restoring photosensitivity to a retina of a subject suffering from vision loss or blindness, said method comprising:
(a) delivering to the retina of said subject a vector comprising the polynucleotide sequence that encodes a peptide sequence of SEQ ID NO: 1 or SEQ ID NO: 3, operatively linked to a heterologous promoter sequence which encodes a rhodopsin domain of a channelrhodopsin expressible in a retinal neuron; and
(b) expressing said vector in said retinal neuron, wherein the expressed rhodopsin renders said retinal neuron photosensitive, and restores photosensitivity to said retina or a portion thereof.

14. The method of claim 12, wherein said subject is mammalian.

15. The method of claim 12, wherein said subject is human.

16. The method of claim 12, wherein said delivering comprises a pharmaceutically acceptable carrying agent.
